(19) United States
(12) Patent Application Publication
(10) Pub. No.: US 2020/0378970 A1
(43) Pub. Date: Dec. 3, 2020

(54) COMPANION DIAGNOSTIC FOR HTRA1 RNA ANTAGONISTS

(71) Applicant: Hoffmann-La Roche Inc., Nutley, NJ (US)

(72) Inventors: Rubén Alvarez Sánchez, Rosenau (FR); Roberto Iacone, Basel (CH); Peter Jakob, Ettingen, Opfikon/zh (CH); Jean-Luc Mary, Huningue (FR); Heidi Rye Hudlebusch, Brønshøj (DK); Thomas Peter John Dunkley, Bottmingen BL (CH); Corinne Stucki, Binningen (CH); Ulrich Friedrich Oskar Luhmann, Riehen (CH); Carolin Rakebrandt, Allschwil (CH)

(21) Appl. No.: 16/766,497

(22) PCT Filed: Dec. 19, 2018
(86) PCT No.: PCT/EP2018/085721

§ 371 Date: May 22, 2020

(30) Foreign Application Priority Data
Dec. 21, 2017 (EP) 17209535.8
Nov. 30, 2018 (EP) 18209473.0

(51) Int. Cl. G01N 33/573; C12N 9/50; G01N 33/50; G01N 33/68

(52) U.S. Cl. CPC G01N 33/573; C12N 9/50; G01N 33/5044; G01N 33/6848; C12Y 304/21108; C12N 2310/3341; G01N 2800/16; G01N 2800/52; G01N 33/6893

(57) ABSTRACT

The present invention relates to the use of HTRA1 mRNA antagonists in the treatment of eye disorders, such as macular degeneration, and the use of an HTRA1 levels in the aqueous and vitreous humor as a diagnostic biomarker for the suitability of treatment of a subject with an HTRA1 mRNA antagonist.

### FIELD OF INVENTION

The present invention relates to the use of HTRA1 mRNA antagonists in the treatment of eye disorders, such as macular degeneration, and the use of an HTRA1 levels in the aqueous and vitreous humor as a diagnostic biomarker for the suitability of treatment of a subject with an HTRA1 mRNA antagonist.

### BACKGROUND

The human high temperature requirement A (HTRA) family of serine proteases are ubiquitously expressed PDZ-proteases that are involved in maintaining protein homeostasis in extracellular compartments by combining the dual functions of a protease and a chaperone. HTRA proteases are implicated in organization of the extracellular matrix, cell proliferation and ageing. Modulation of HTRA activity is connected with severe diseases, including Duchenne muscular dystrophy (Bakay et al. 2002, Neuromuscul. Disord. 12: 125-141), arthritis, such as osteoarthritis (Grau et al. 2006, JBC 281: 6124-6129), cancer, familial ischemic cerebral small-vessel disease and age-related macular degeneration, as well as Parkinson's disease and Alzheimer's disease. The human HTRA1 contains an insulin-like growth factor (IGF) binding domain. It has been proposed to regulate IGF availability and cell growth (Zumbrunn and Trueb, 1996, FEES Letters 398:189-192) and to exhibit tumor suppressor properties. HTRA1 expression is down-regulated in metastatic melanoma, and may thus indicate the degree of melanoma progression. Overexpression of HTRA1 in a metastatic melanoma cell line reduced proliferation and invasion in vitro, and reduced tumor growth in a xenograft mouse model (Baldi et al., 2002, Oncogene 21:6684-6688). HTRA1 expression is also down-regulated in ovarian cancer. In ovarian cancer cell lines, HTRA1 overexpression induces cell death, while antisense HTRA1 expression promoted anchorage-independent growth (Chien et al., 2004, Oncogene 23:1636-1644).

In addition to its effect on the IGF pathway, HTRA1 also inhibits signaling by the TGFβ family of growth factors (Oka et al., 2004, Development 131:1041-1053). HTRA1 can cleave amyloid precursor protein (APP), and HTRA1 inhibitors cause the accumulation of Aβ peptide in cultured cells. Thus, HTRA1 is also implicated in Alzheimer's disease (Grau et al., 2005, Proc. Nat. Acad. Sci. USA. 102:6021-6026).

Furthermore, HTRA1 upregulation has been observed and seems to be associated to Duchenne muscular dystrophy (Bakay et al. 2002, Neuromuscul. Disord. 12: 125-141) and osteoarthritis (Grau et al. 2006, JBC 281: 6124-6129) and AMD (Fritsche, et al. Nat Gen 2013 45(4):433-9.)

A single nucleotide polymorphism (SNP) in the HTRA1 promoter region (rs11200638) is associated with a 10 fold increased the risk of developing age-related macular degeneration (AMD). Moreover the HTRA1 SNPs are in linkage disequilibrium with the ARMS2 SNP (rs10490924) associated with increased risk of developing age-related macular degeneration (AMD). The risk allele is associated with 2-3 fold increased HTRA1 mRNA and protein expression, and HTRA1 is present in drusen in patients with AMD (Dewan et al., 2006, Science 314:989-992; Yang et al., 2006, Science 314:992-993). Over-expression of HtrA1 Induces AMD-like phenotype in mice. The hHTRA transgenic mouse (Veierkottn, PlosOne 2011) reveals degradation of the elastic lamina of Bruch's membrane, determines choroidal vascular abnormalities (Jones, PNAS 2011) and increases the Polypoidal choroidal vasculopathy (PCV) lesions (Kumar, IOVS 2014). Additionally it has been reported that Bruch's membrane damage in hHTRA1 Tg mice, which determines upon exposure to cigarette smoke 3 fold increases CNV (Nakayama, IOVS 2014)

Age-related macular degeneration (AMD) is the leading cause of irreversible loss of vision in people over the age of 65. With onset of AMD there is gradual loss of the light sensitive photoreceptor cells in the back of the eye, the underlying pigment epithelial cells that support them metabolically, and the sharp central vision they provide. Age is the major risk factor for the onset of AMD: the likelihood of developing AMD triples after age 55. Smoking, light iris color, sex (women are at greater risk), obesity, and repeated exposure to UV radiation also increase the risk of AMD. AMD progression can be defined in three stages: 1) early, 2) intermediate, and 3) advanced AMD. There are two forms of advanced AMD: dry AMD (also called geographic atrophy, GA) and wet AMD (also known as exudative AMD). Dry AMD is characterized by loss of photoreceptors and retinal pigment epithelium cells, leading to visual loss. Wet AMD, is associated with pathologic choroidal (also referred to as subretinal) neovascularization. Leakage from abnormal blood vessels forming in this process damages the macula and impairs vision, eventually leading to blindness. In some cases, patients can present pathologies associated with both types of advanced AMD. Treatment strategies for wet AMD require frequent injections into the eye and are focused mainly on delaying the disease progression. Currently no approved treatment is available for dry AMD. WO2017/075212 relates generally to anti-HtrA1 antibodies and methods of using the same, including the use of an anti-Htra1 antibody as a biomarker for selection of patients.

Tosi et al., IOVS 20017, p 162-167 reports of elevated HTRA1 concentration in aqueous humor of patients with neovascular age-related macular degeneration, and that the levels of HTRA1 in aqueous humor are normalized after treatment with ranibizumab, and antibody which targets VEGF-A.

WO 2008/013893 claims a composition for treating a subject suffering from age related macular degeneration comprising a nucleic acid molecules comprising an antisense sequence that hybridizes to HTRA1 gene or mRNA: No antisense molecules are disclosed. WO2009/006460 provides siRNAs targeting HTRA1 and their use in treating AMD. PCT/EP2017/065937 and EP17173964.2, both of which are incorporated by reference in their entirety, disclose antisense oligonucleotides which are potent in vivo inhibitors of HTRA1 mRNA and their therapeutic use, including use to treat macular degeneration.

### SUMMARY OF THE INVENTION

The inventors have determined that there is a direct correlation between the inhibition of HTRA1 mRNA in the retinal epithelial cells from subjects treated with HTRA1 mRNA antagonists, and the level of HTRA1 protein in the aqueous and vitreous humor of the subjects. The direct correlation allows for the use of HTRA1 as a biomarker for diagnostic or prognostic use to determine the suitability of a subject for treatment with an HTRA1 mRNA antagonist, as well as a companion diagnostic with HTRA1 mRNA antagonist therapeutics, for example in patient monitoring.

The invention provides for a method for determining the suitability of treatment of a subject for administration with an HTRA1 mRNA antagonist, said method comprising the steps of:

i) determining the level of HTRA1 in a sample of aqueous or vitreous humor obtained from the subject

ii) comparing the level of HTRA1 obtained from step i) with one or more reference samples or reference values;

to determine whether the subject is likely to be, or is suitable for, treatment of with the HTRA1 mRNA antagonist,

wherein the subject is suffering from or is at risk of developing an ocular disorder, such as macular degeneration.

The invention provides for a diagnostic or prognostic method for determining the suitability of treatment of a subject for administration with an HTRA1 mRNA antagonist, said method comprising the steps of:

i) determining the level of HTRA1 in a sample of aqueous or vitreous humor obtained from the subject

ii) comparing the level of HTRA1 obtained from step i) with one or more reference samples or reference values;

to determine whether the subject is likely to be, or is suitable for, treatment of with the HTRA1 mRNA antagonist,

wherein the subject is suffering from or is at risk of developing an ocular disorder, such as macular degeneration.

The invention provides for a method determining the suitable dose regimen for—an HTRA1 mRNA antagonist, to a subject in need to treatment with the HTRA1 mRNA antagonist, said method comprising the steps of:

i) determining the level of HTRA1 in a sample of aqueous or vitreous humor obtained from the subject

ii) comparing the level of HTRA1 obtained from step i) with one or more reference samples or reference values;

to determine whether the subject is likely to be, or is suitable for, treatment of with the HTRA1 mRNA antagonist,

wherein the subject is suffering from or is at risk of developing an ocular disorder, such as macular degeneration.

Advantageously, the sample obtained from the subject referred to in the method or use of the invention is a sample of the subjects aqueous humor.

The invention provides for a method of treatment of a subject who is in need of treatment with an HTRA1 mRNA antagonist, for example a subject who is suffering from or is at risk of developing macular degeneration, said method comprising:
i) determining the level of HTRA1 in a sample of aqueous or vitreous humor obtained from the subject
ii) comparing the level of HTRA1 obtained from step i) with one or more reference samples or reference values;
iii) to determine whether the subject is likely to be, or is suitable for, treatment of with the HTRA1 mRNA antagonist,
iv) administering an effective amount of a HTRA1 mRNA antagonist to the subject.

The invention provides for the use of an HTRA1 antibody as a companion diagnostic for a HTRA1 mRNA antagonist therapeutic.

The invention provides for the use of an HTRA1 antibody in the detection of HTRA1 levels in an aqueous humor or vitreous humor sample obtained from a subject who is undergoing treatment with an HTRA1 mRNA antagonist, or is being assessed for suitability of treatment with an HTRA1 mRNA antagonist.

The invention provides for the use of a biomarker for determining the likely response of a subject to a therapeutic agent comprising an HTRA1 mRNA antagonist, wherein the biomarker comprises an elevated level of HTRA1 in a biological sample obtained from the aqueous humor or vitreous humor of the subject, as compared to the level of HTRA1 obtained from a reference sample from a healthy subject.

### BRIEF DESCRIPTION OF FIGURES

FIG. 1: An exemplary LNA antisense oligonucleotide antagonist of HTRA1 (SEQ ID NO 13, Compound ID #13.1)

FIG. 2: An exemplary LNA antisense oligonucleotide antagonist of HTRA1 (SEQ ID NO 15, Compound ID #15.3)

FIG. 3: An exemplary LNA antisense oligonucleotide antagonist of HTRA1 (SEQ ID 17, Compound ID #17)

FIG. 4: An exemplary LNA antisense oligonucleotide antagonist of HTRA1 (SEQ ID NO 18, Compound ID #18)

FIG. 5: An exemplary LNA antisense oligonucleotide antagonist of HTRA1 (SEQ ID NO 19, Compound ID #19)

FIG. 6: Non-Human Primate (NHP) PK/PD study using the compound shown in FIG. 2 (compound ID #15.3)—see example 1, IVT administration, 25 μg/eye. A) HTRA1 mRNA level measured in the retina by qPCR. B) HTRA1 mRNA level illustrated by ISH. C-D) Quantification of HTRA1 protein level in retina and vitreous, respectively, by IP-MS. Dots show data for individual animals. Error bars show standard deviations for technical replicates (n=3).

FIG. 7. NHP PK/PD study (Example 2) using the compounds shown in FIG. 3 (Compound ID #73.1), 4 (Compound ID #86), and 5 (Compound ID #67), IVT administration, 25 μg/eye. A) HTRA1 mRNA level measured in the retina by qPCR. B) oligo content in the retina measured by oligo ELISA. C) HTRA1 mRNA level illustrated by ISH. D-E) Quantification of HTRA1 protein level in retina and vitreous, respectively, by IP-MS. Dots show data for individual animals. Error bars show standard errors for technical replicates (n=3). F-G) Reduction in HTRA1 protein level in retina and vitreous, respectively illustrated by western blot.

FIG. 8. HTRA1 Protein Quantification—Immunocapture and LC-MS Approach. 8A schematic of the approach, 8B quantification by LC-MS of HTRA protein levels in retina, vitreous and aqueous humor after administration of LNA antisense compounds targeting HTRA1 mRNA. 8C HTRA1 protein reduction in retina, vitreous and aqueous humor. 8D. PD Analysis in serial aqueous humor samples taken 2, 7, 14 and 21 days after a single IVT 25 μg dose.

FIG. 9A. Compounds #15.3 and #17 were administered intravitreally in cynomolgus monkeys, and aqueous humor samples were collected at days 3, 8, 15, and 22 post-injection. Proteins from undiluted samples were analyzed by capillary electrophoresis using a Peggy Sue device (Protein Simple). HTRA1 was detected using a custom-made polycolonal rabbit antiserum. Data from animals #J60154 (Vehicle), J60158 (C. Id #15.3), J60162 (C. Id #17) are presented.

FIG. 9B. Signal intensities were quantified by comparison to purified recombinant (S328A mutant) HTRA1 protein (Origene, #TP700208). The calibration curve is shown here.

FIG. 9C. Top panel: Calculated HTRA1 aqueous humor concentration from individual animal was plotted against time post injection. Bottom panel: average HTRA1 concentration for the vehicle group at each time point was determined and corresponding relative concentration in treated animals calculated. Open circle: individual value, closed circle: group average. % HTRA1 reduction for day 22 is indicated.

FIG. 10. HTRA1 mRNA plotted against HTRA1 protein levels in aqueous humor (blue diamonds) or in retina (red squares) in cynomolgus monkeys treated with various LNA molecules targeting the HTRA1 transcript. Values are expressed as percentage normalized to PBS controls.

FIG. 11. Correlation of HTRA1 protein in aqueous humor with (A) HTRA1 protein in retina and (B) HTRA1 mRNA in retina in cynomolgus monkeys treated with various LNA molecules targeting the HTRA1 transcript. Values are expressed as percentage normalized to PBS controls.

FIG. 12A. Average HTRA1 protein levels day 36 post IVT LNA application. Percent HTRA1 suppressions indicated in blue. Error bars: group sd.

FIG. 12B. HTRA1 concentrations in posterior ocular tissues vs aqueous humor (AH) in control (∘) and LNA treated (●) animals.

FIG. 13. Dynamic of HTRA1 concentration in Aqueous humor.

Filled symbol: active treatment, empty symbols: vehicle treatment.

Individual normalized values.

FIG. 14. HTRA1 mRNA expression levels in the different compartments with and without LNA treatment. HTRA1 mRNA levels are shown as reads per kilobase per million sequencing reads (RPKM). Error bars represent the standard deviation around the mean RPKM from at least 3 animals. Asterisks represent statistical significance (false discovery rate <0.05). The numbers above the bars show percent decrease of HTRA1 mRNA levels.

### DEFINITIONS & DETAILED DESCRIPTION OF THE INVENTION

In the present description the term “alkyl”, alone or in combination, signifies a straight-chain or branched-chain alkyl group with 1 to 8 carbon atoms, particularly a straight or branched-chain alkyl group with 1 to 6 carbon atoms and more particularly a straight or branched-chain alkyl group with 1 to 4 carbon atoms. Examples of straight-chain and branched-chain C₁-C₈ alkyl groups are methyl, ethyl, propyl, isopropyl, butyl, isobutyl, tert.-butyl, the isomeric pentyls, the isomeric hexyls, the isomeric heptyls and the isomeric octyls, particularly methyl, ethyl, propyl, butyl and pentyl. Particular examples of alkyl are methyl, ethyl and propyl.

The term “cycloalkyl”, alone or in combination, signifies a cycloalkyl ring with 3 to 8 carbon atoms and particularly a cycloalkyl ring with 3 to 6 carbon atoms. Examples of cycloalkyl are cyclopropyl, cyclobutyl, cyclopentyl, cyclohexyl, cycloheptyl and cyclooctyl, more particularly cyclopropyl and cyclobutyl. A particular example of “cycloalkyl” is cyclopropyl.

The term “alkoxy”, alone or in combination, signifies a group of the formula alkyl-O- in which the term “alkyl” has the previously given significance, such as methoxy, ethoxy, n-propoxy, isopropoxy, n-butoxy, isobutoxy, sec.butoxy and tert.butoxy. Particular “alkoxy” are methoxy and ethoxy. Methoxyethoxy is a particular example of “alkoxyalkoxy”.

The term “oxy”, alone or in combination, signifies the —O— group.

The term “alkenyl”, alone or in combination, signifies a straight-chain or branched hydrocarbon residue comprising an olefinic bond and up to 8, preferably up to 6, particularly preferred up to 4 carbon atoms. Examples of alkenyl groups are ethenyl, 1-propenyl, 2-propenyl, isopropenyl, 1-butenyl, 2-butenyl, 3-butenyl and isobutenyl.

The term “alkynyl”, alone or in combination, signifies a straight-chain or branched hydrocarbon residue comprising a triple bond and up to 8, preferably up to 6, particularly preferred up to 4 carbon atoms.

The terms “halogen” or “halo”, alone or in combination, signifies fluorine, chlorine, bromine or iodine and particularly fluorine, chlorine or bromine, more particularly fluorine. The term “halo”, in combination with another group, denotes the substitution of said group with at least one halogen, particularly substituted with one to five halogens, particularly one to four halogens, i.e. one, two, three or four halogens.

The term “haloalkyl”, alone or in combination, denotes an alkyl group substituted with at least one halogen, particularly substituted with one to five halogens, particularly one to three halogens. Examples of haloalkyl include monofluoro-, difluoro- or trifluoro-methyl, -ethyl or -propyl, for example 3,3,3-trifluoropropyl, 2-fluoroethyl, 2,2,2-trifluoroethyl, fluoromethyl or trifluoromethyl. Fluoromethyl, difluoromethyl and trifluoromethyl are particular “haloalkyl”.

The term “halocycloalkyl”, alone or in combination, denotes a cycloalkyl group as defined above substituted with at least one halogen, particularly substituted with one to five halogens, particularly one to three halogens. Particular example of “halocycloalkyl” are halocyclopropyl, in particular fluorocyclopropyl, difluorocyclopropyl and trifluorocyclopropyl.

The terms “hydroxyl” and “hydroxy”, alone or in combination, signify the —OH group.

The terms “thiohydroxyl” and “thiohydroxy”, alone or in combination, signify the —SH group.

The term “carbonyl”, alone or in combination, signifies the —C(O)— group.

The term “carboxy” or “carboxyl”, alone or in combination, signifies the —COOH group.

The term “amino”, alone or in combination, signifies the primary amino group (—NH₂), the secondary amino group (—NH—), or the tertiary amino group (—N—).

The term “alkylamino”, alone or in combination, signifies an amino group as defined above substituted with one or two alkyl groups as defined above.

The term “sulfonyl”, alone or in combination, means the —SO₂ group.

The term “sulfinyl”, alone or in combination, signifies the —SO— group.

The term “sulfonyl”, alone or in combination, signifies the —S— group.

The term “cyano”, alone or in combination, signifies the —CN group.

The term “azido”, alone or in combination, signifies the —N₃ group.

The term “nitro”, alone or in combination, signifies the NO₂ group.

The term “formyl”, alone or in combination, signifies the —C(O)H group.

The term “carbamoyl”, alone or in combination, signifies the —C(O)NH₂ group.

The term “cabamido”, alone or in combination, signifies the —NH—C(O)—NH₂ group.

The term “aryl”, alone or in combination, denotes a monovalent aromatic carbocyclic mono- or bicyclic ring system comprising 6 to 10 carbon ring atoms, optionally substituted with 1 to 3 substituents independently selected from halogen, hydroxyl, alkyl, alkenyl, alkynyl, alkoxy, alkoxyalkyl, alkenyloxy, carboxyl, alkoxycarbonyl, alkylcarbonyl and formyl. Examples of aryl include phenyl and naphthyl, in particular phenyl.

The term “heteroaryl”, alone or in combination, denotes a monovalent aromatic heterocyclic mono- or bicyclic ring system of 5 to 12 ring atoms, comprising 1, 2, 3 or 4 heteroatoms selected from N, O and S, the remaining ring atoms being carbon, optionally substituted with 1 to 3 substituents independently selected from halogen, hydroxyl, alkyl, alkenyl, alkynyl, alkoxy, alkoxyalkyl, alkenyloxy, carboxyl, alkoxycarbonyl, alkylcarbonyl and formyl. Examples of heteroaryl include pyrrolyl, furanyl, thienyl, imidazolyl, oxazolyl, thiazolyl, triazolyl, oxadiazolyl, thiadiazolyl, tetrazolyl, pyridinyl, pyrazinyl, pyrazolyl, pyridazinyl, pyrimidinyl, triazinyl, azepinyl, diazepinyl, isoxazolyl, benzofuranyl, isothiazolyl, benzothienyl, indolyl, isoindolyl, isobenzofuranyl, benzimidazolyl, benzoxazolyl, benzoisoxazolyl, benzothiazolyl, benzoisothiazolyl, benzooxadiazolyl, benzothiadiazolyl, benzotriazolyl, purinyl, quinolinyl, isoquinolinyl, quinazolinyl, quinoxalinyl, carbazolyl or acridinyl.

The term “heterocyclyl”, alone or in combination, signifies a monovalent saturated or partly unsaturated mono- or bicyclic ring system of 4 to 12, in particular 4 to 9 ring atoms, comprising 1, 2, 3 or 4 ring heteroatoms selected from N, O and S, the remaining ring atoms being carbon, optionally substituted with 1 to 3 substituents independently selected from halogen, hydroxyl, alkyl, alkenyl, alkynyl, alkoxy, alkoxyalkyl, alkenyloxy, carboxyl, alkoxycarbonyl, alkylcarbonyl and formyl. Examples for monocyclic saturated heterocyclyl are azetidinyl, pyrrolidinyl, tetrahydrofuranyl, tetrahydro-thienyl, pyrazolidinyl, imidazolidinyl, oxazolidinyl, isoxazolidinyl, thiazolidinyl, piperidinyl, tetrahydropyranyl, tetrahydrothiopyranyl, piperazinyl, morpholinyl, thiomorpholinyl, 1,1-dioxo-thiomorpholin-4-yl, azepanyl, diazepanyl, homopiperazinyl, or oxazepanyl. Examples for bicyclic saturated heterocycloalkyl are 8-aza-bicyclo[3.2.1]octyl, quinuclidinyl, 8-oxa-3-aza-bicyclo[3.2.1]octyl, 9-aza-bicyclo[3.3.1]nonyl, 3-oxa-9-aza-bicyclo[3.3.1]nonyl, or 3-thia-9-aza-bicyclo[3.3.1]nonyl. Examples for partly unsaturated heterocycloalkyl are dihydrofuryl, imidazolinyl, dihydro-oxazolyl, tetrahydro-pyridinyl or dihydropyranyl.

The term “pharmaceutically acceptable salts” refers to those salts which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid, particularly hydrochloric acid, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein. In addition these salts may be prepared form addition of an inorganic base or an organic base to the free acid. Salts derived from an inorganic base include, but are not limited to, the sodium, potassium, lithium, ammonium, calcium, magnesium salts. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyamine resins. The oligonucleotide of the invention can also be present in the form of zwitterions. Particularly preferred pharmaceutically acceptable salts of the invention are the salts of hydrochloric acid, hydrobromic acid, sulfuric acid, phosphoric acid and methanesulfonic acid.

The term “protecting group”, alone or in combination, signifies a group which selectively blocks a reactive site in a multifunctional compound such that a chemical reaction can be carried out selectively at another unprotected reactive site. Protecting groups can be removed. Exemplary protecting groups are amino-protecting groups, carboxy-protecting groups or hydroxy-protecting groups.

“Hydroxyl protecting group” is a protecting group of the hydroxyl group and is also used to protect thiol groups. Examples of hydroxyl protecting groups are acetyl (Ac), benzoyl (Bz), benzyl (Bn), β-methoxyethoxymethyl ether (MEM), dimethoxytrityl (or bis-(4-methoxyphenyl)phenylmethyl) (DMT), trimethoxytrityl (or tris-(4-methoxyphenyl)phenylmethyl) (TMT), methoxymethyl ether (MOM), methoxytrityl [(4-methoxyphenyl)diphenylmethyl (MMT), p-methoxybenzyl ether (PMB), methylthiomethyl ether, pivaloyl (Piv), tetrahydropyranyl (THP), tetrahydrofuran (THF), trityl or triphenylmethyl (Tr), silyl ether (for example trimethylsilyl (TMS), tert-butyldimethylsilyl (TBDMS), tri-iso-propylsilyloxymethyl (TOM) and triisopropylsilyl (TIPS) ethers), methyl ethers and ethoxyethyl ethers (EE). Particular examples of hydroxyl protecting group are DMT and TMT, in particular DMT.

If one of the starting materials or compounds of the invention contain one or more functional groups which are not stable or are reactive under the reaction conditions of one or more reaction steps, appropriate protecting groups (as described e.g. in “Protective Groups in Organic Chemistry” by T. W. Greene and P. G. M. Wuts, 3^{rd} Ed., 1999, Wiley, New York) can be introduced before the critical step applying methods well known in the art. Such protecting groups can be removed at a later stage of the synthesis using standard methods described in the literature. Examples of protecting groups are tert-butoxycarbonyl (Boc), 9-fluorenylmethyl carbamate (Fmoc), 2-trimethylsilylethyl carbamate (Teoc), carbobenzyloxy (Cbz) and p-methoxybenzyloxycarbonyl (Moz).

The compounds described herein can contain several asymmetric centers and can be present in the form of optically pure enantiomers, mixtures of enantiomers such as, for example, racemates, mixtures of diastereoisomers, diastereoisomeric racemates or mixtures of diastereoisomeric racemates.

An “effective amount” of an agent, e.g., a pharmaceutical formulation, refers to an amount effective, at dosages and for periods of time necessary, to achieve the desired therapeutic or prophylactic result.

Oligonucleotide

The term “oligonucleotide” as used herein is defined as it is generally understood by the skilled person as a molecule comprising two or more covalently linked nucleosides. Such covalently bound nucleosides may also be referred to as nucleic acid molecules or oligomers. Oligonucleotides are commonly made in the laboratory by solid-phase chemical synthesis followed by purification. When referring to a sequence of the oligonucleotide, reference is made to the sequence or order of nucleobase moieties, or modifications thereof, of the covalently linked nucleotides or nucleosides. An oligonucleotide is man-made, and is chemically synthesized, and is typically purified or isolated. The oligonucleotide may comprise one or more modified nucleosides or nucleotides.

The HTRA1 RNA antagonists referred to in the present invention may be oligonucleotides, such as siRNAs or antisense oligonucleotides, which are capable of modulating the expression of the HTRA1 target nucleic acid. A modulation via a HTRA1 RNA antagonist includes the ability to inhibit, down-regulate, reduce, suppress, remove, stop, block, prevent, lessen, lower, avoid or terminate expression of HTRA1, e.g. by degradation of mRNA or blockage of transcription or via alternative splicing of the HTRA1 pre-mRNA (splice modulation of HTRA1 pre-mRNA).

siRNAs and shRNAs

In some embodiments, the HTRA1 mRNA antagonist is an siRNA or a shRNA.

An siRNA is a short double stranded complex, comprising a sense and antisense strand which together form a duplex region of 18-25 base pairs. The antisense strand of siRNAs which target HTRA1 are complementary to the HTRA1 target nucleic acid, such as the HTRA1 mRNA. US2007185317 discloses siRNAs targeting HTRA1.

A short hairpin RNA or small hairpin RNA (shRNA/Hairpin Vector) is an artificial RNA molecule with a tight hairpin turn that can be used to silence target gene expression via RNA interference (RNAi). Expression of shRNA in cells is typically accomplished by delivery of plasmids or through viral or bacterial vectors.

Antisense Oligonucleotides

Advantageously, the HTRA1 mRNA antagonist is an antisense oligonucleotide which targets an HTRA1 nucleic acid.

The term “Antisense oligonucleotide” as used herein is defined as oligonucleotides capable of modulating expression of a target gene by hybridizing to a target nucleic acid, in particular to a contiguous sequence on a target nucleic acid, in a cell which is expressing the target nucleic acid. The antisense oligonucleotides are not essentially double stranded and are therefore not siRNAs or shRNAs. Preferably, the antisense oligonucleotides of the present invention are single stranded. It is understood that single stranded oligonucleotides of the present invention can form hairpins or intermolecular duplex structures (duplex between two molecules of the same oligonucleotide), as long as the degree of intra or inter self-complementarity is less than 50% across of the full length of the oligonucleotide. The antisense oligonucleotide of the invention is man-made, and is chemically synthesized, and is typically purified or isolated. The antisense oligonucleotide of the invention may comprise one or more modified nucleosides or nucleotides.

Contiguous Nucleotide Sequence

The term “contiguous nucleotide sequence” refers to the region of the oligonucleotide which is complementary to the target nucleic acid. The term is used interchangeably herein with the term “contiguous nucleobase sequence” and the term “oligonucleotide motif sequence”. In some embodiments all the nucleotides of the oligonucleotide constitute the contiguous nucleotide sequence. In some embodiments the oligonucleotide comprises the contiguous nucleotide sequence, such as a F-G-F′ gapmer region, and may optionally comprise further nucleotide(s), for example a nucleotide linker region which may be used to attach a functional group to the contiguous nucleotide sequence. The nucleotide linker region may or may not be complementary to the target nucleic acid.

Nucleotides

Nucleotides are the building blocks of oligonucleotides and polynucleotides, and for the purposes of the present invention include both naturally occurring and non-naturally occurring nucleotides. In nature, nucleotides, such as DNA and RNA nucleotides comprise a ribose sugar moiety, a nucleobase moiety and one or more phosphate groups (which is absent in nucleosides). Nucleosides and nucleotides may also interchangeably be referred to as “units” or “monomers”.

Modified Nucleoside

The term “modified nucleoside” or “nucleoside modification” as used herein refers to nucleosides modified as compared to the equivalent DNA or RNA nucleoside by the introduction of one or more modifications of the sugar moiety or the (nucleo)base moiety. In a preferred embodiment the modified nucleoside comprise a modified sugar moiety. The term modified nucleoside may also be used herein interchangeably with the term “nucleoside analogue” or modified “units” or modified “monomers”. Nucleosides with an unmodified DNA or RNA sugar moiety are termed DNA or RNA nucleosides herein. Nucleosides with modifications in the base region of the DNA or RNA nucleoside are still generally termed DNA or RNA if they allow Watson Crick base pairing.

Modified Internucleoside Linkage

The term “modified internucleoside linkage” is defined as generally understood by the skilled person as linkages other than phosphodiester (PO) linkages, that covalently couples two nucleosides together. The oligonucleotides of the invention may therefore comprise modified internucleoside linkages. In some embodiments, the modified internucleoside linkage increases the nuclease resistance of the oligonucleotide compared to a phosphodiester linkage. For naturally occurring oligonucleotides, the internucleoside linkage includes phosphate groups creating a phosphodiester bond between adjacent nucleosides. Modified internucleoside linkages are particularly useful in stabilizing oligonucleotides for in vivo use, and may serve to protect against nuclease cleavage at regions of DNA or RNA nucleosides in the oligonucleotide of the invention, for example within the gap region of a gapmer oligonucleotide, as well as in regions of modified nucleosides, such as region F and F′.

In an embodiment, the oligonucleotide comprises one or more internucleoside linkages modified from the natural phosphodiester, such one or more modified internucleoside linkages that is for example more resistant to nuclease attack. Nuclease resistance may be determined by incubating the oligonucleotide in blood serum or by using a nuclease resistance assay (e.g. snake venom phosphodiesterase (SVPD)), both are well known in the art. Internucleoside linkages which are capable of enhancing the nuclease resistance of an oligonucleotide are referred to as nuclease resistant internucleoside linkages. In some embodiments at least 50% of the internucleoside linkages in the oligonucleotide, or contiguous nucleotide sequence thereof, are modified, such as at least 60%, such as at least 70%, such as at least 75% such as at least 80% or such as at least 90% of the internucleoside linkages in the oligonucleotide, or contiguous nucleotide sequence thereof, are nuclease resistant internucleoside linkages. In some embodiments all of the internucleoside linkages of the oligonucleotide, or contiguous nucleotide sequence thereof, are nuclease resistant internucleoside linkages. It will be recognized that, in some embodiments the nucleosides which link the oligonucleotide of the invention to a non-nucleotide functional group, such as a conjugate, may be phosphodiester. A preferred modified internucleoside linkage for use in the oligonucleotide of the invention is phosphorothioate.

Phosphorothioate internucleoside linkages are particularly useful due to nuclease resistance, beneficial pharmacokinetics and ease of manufacture. In some embodiments at least 50% of the internucleoside linkages in the oligonucleotide, or contiguous nucleotide sequence thereof, are phosphorothioate, such as at least 60%, such as at least 70%, such as at least 75%, such as at least 80% or such as at least 90% of the internucleoside linkages in the oligonucleotide, or contiguous nucleotide sequence thereof, are phosphorothioate. In some embodiments, all of the internucleoside linkages of the oligonucleotide, or contiguous nucleotide sequence thereof, are phosphorothioate. In some embodiments, the oligonucleotide of the invention comprises both phosphorothioate internucleoside linkages and at least one phosphodiester linkage, such as 2, 3 or 4 phosphodiester linkages, in addition to the phosphorodithioate linkage(s). In a gapmer oligonucleotide, phosphodiester linkages, when present, are suitably not located between contiguous DNA nucleosides in the gap region G.

Nuclease resistant linkages, such as phosphorothioate linkages, are particularly useful in oligonucleotide regions capable of recruiting nuclease when forming a duplex with the target nucleic acid, such as region G for gapmers. Phosphorothioate linkages may, however, also be useful in non-nuclease recruiting regions and/or affinity enhancing regions such as regions F and F′ for gapmers. Gapmer oligonucleotides may, in some embodiments comprise one or more phosphodiester linkages in region F or F′, or both region F and F′, where all the internucleoside linkages in region G may be phosphorothioate.

Advantageously, all the internucleoside linkages in the contiguous nucleotide sequence of the oligonucleotide, or all the internucleoside linkages of the oligonucleotide, are phosphorothioate linkages.

It is recognized that, as disclosed in EP 2 742 135, antisense oligonucleotides may comprise other internucleoside linkages (other than phosphodiester and phosphorothioate), for example alkyl phosphonate/methyl phosphonate internucleosides, which according to EP 2 742 135 may for example be tolerated in an otherwise DNA phosphorothioate the gap region.

Nucleobase

The term nucleobase includes the purine (e.g. adenine and guanine) and pyrimidine (e.g. uracil, thymine and cytosine) moiety present in nucleosides and nucleotides which form hydrogen bonds in nucleic acid hybridization. In the context of the present invention the term nucleobase also encompasses modified nucleobases which may differ from naturally occurring nucleobases, but are functional during nucleic acid hybridization. In this context “nucleobase” refers to both naturally occurring nucleobases such as adenine, guanine, cytosine, thymidine, uracil, xanthine and hypoxanthine, as well as non-naturally occurring variants. Such variants are for example described in Hirao et al (2012) Accounts of Chemical Research vol 45 page 2055 and Bergstrom (2009) Current Protocols in Nucleic Acid Chemistry Suppl. 37 1.4.1.

In a some embodiments the nucleobase moiety is modified by changing the purine or pyrimidine into a modified purine or pyrimidine, such as substituted purine or substituted pyrimidine, such as a nucleobased selected from isocytosine, pseudoisocytosine, 5-methyl cytosine, 5-thiozolo-cytosine, 5-propynyl-cytosine, 5-propynyl-uracil, 5-bromouracil 5-thiazolo-uracil, 2-thio-uracil, 2′thio-thymine, inosine, diaminopurine, 6-aminopurine, 2-aminopurine, 2,6-diaminopurine and 2-chloro-6-aminopurine.

The nucleobase moieties may be indicated by the letter code for each corresponding nucleobase, e.g. A, T, G, C or U, wherein each letter may optionally include modified nucleobases of equivalent function. For example, in the exemplified oligonucleotides, the nucleobase moieties are selected from A, T, G, C, and 5-methyl cytosine. Optionally, for LNA gapmers, 5-methyl cytosine LNA nucleosides may be used.

Modified Oligonucleotide

The term modified oligonucleotide describes an oligonucleotide comprising one or more sugar-modified nucleosides and/or modified internucleoside linkages. The term chimeric” oligonucleotide is a term that has been used in the literature to describe oligonucleotides with modified nucleosides.

Complementarity

The term “complementarity” describes the capacity for Watson-Crick base-pairing of nucleosides/nucleotides. Watson-Crick base pairs are guanine (G)-cytosine (C) and adenine (A)-thymine (T)/uracil (U). It will be understood that oligonucleotides may comprise nucleosides with modified nucleobases, for example 5-methyl cytosine is often used in place of cytosine, and as such the term complementarity encompasses Watson Crick base-paring between non-modified and modified nucleobases (see for example Hirao et al (2012) Accounts of Chemical Research vol 45 page 2055 and Bergstrom (2009) Current Protocols in Nucleic Acid Chemistry Suppl. 37 1.4.1).

The term “% complementary” as used herein, refers to the proportion of nucleotides (in percent) of a contiguous nucleotide sequence in a nucleic acid molecule (e.g. oligonucleotide) which across the contiguous nucleotide sequence, are complementary to a reference sequence (e.g. a target sequence or sequence motif). The percentage of complementarity is thus calculated by counting the number of aligned nucleobases that are complementary (from Watson Crick base pair) between the two sequences (when aligned with the target sequence 5′-3′ and the oligonucleotide sequence from 3′-5′), dividing that number by the total number of nucleotides in the oligonucleotide and multiplying by 100. In such a comparison a nucleobase/nucleotide which does not align (form a base pair) is termed a mismatch. Insertions and deletions are not allowed in the calculation of % complementarity of a contiguous nucleotide sequence. It will be understood that in determining complementarity, chemical modifications of the nucleobases are disregarded as long as the functional capacity of the nucleobase to form Watson Crick base pairing is retained (e.g. 5′-methyl cytosine is considered identical to a cytosine for the purpose of calculating % identity).

The term “fully complementary”, refers to 100% complementarity.

Identity

The term “Identity” as used herein, refers to the proportion of nucleotides (expressed in percent) of a contiguous nucleotide sequence in a nucleic acid molecule (e.g. oligonucleotide) which across the contiguous nucleotide sequence, are identical to a reference sequence (e.g. a sequence motif). The percentage of identity is thus calculated by counting the number of aligned bases that are identical (a match) between two sequences (in the contiguous nucleotide sequence of the compound of the invention and in the reference sequence), dividing that number by the total number of nucleotides in the oligonucleotide and multiplying by 100. Therefore, Percentage of Identity=(Matches×100)/Length of aligned region (e.g. the contiguous nucleotide sequence). Insertions and deletions are not allowed in the calculation the percentage of identity of a contiguous nucleotide sequence. It will be understood that in determining identity, chemical modifications of the nucleobases are disregarded as long as the functional capacity of the nucleobase to form Watson Crick base pairing is retained (e.g. 5-methyl cytosine is considered identical to a cytosine for the purpose of calculating % identity).

Hybridization

The term “hybridizing” or “hybridizes” as used herein is to be understood as two nucleic acid strands (e.g. an oligonucleotide and a target nucleic acid) forming hydrogen bonds between base pairs on opposite strands thereby forming a duplex. The affinity of the binding between two nucleic acid strands is the strength of the hybridization. It is often described in terms of the melting temperature (Tₘ) defined as the temperature at which half of the oligonucleotides are duplexed with the target nucleic acid. At physiological conditions Tₘ is not strictly proportional to the affinity (Mergny and Lacroix, 2003, Oligonucleotides 13:515-537). The standard state Gibbs free energy ΔG° is a more accurate representation of binding affinity and is related to the dissociation constant (K_{d}) of the reaction by ΔG°=−RT ln(K_{d}), where R is the gas constant and T is the absolute temperature. Therefore, a very low ΔG° of the reaction between an oligonucleotide and the target nucleic acid reflects a strong hybridization between the oligonucleotide and target nucleic acid. ΔG° is the energy associated with a reaction where aqueous concentrations are 1M, the pH is 7, and the temperature is 37° C. The hybridization of oligonucleotides to a target nucleic acid is a spontaneous reaction and for spontaneous reactions ΔG° is less than zero. ΔG° can be measured experimentally, for example, by use of the isothermal titration calorimetry (ITC) method as described in Hansen et al., 1965, Chem. Comm. 36-38 and Holdgate et al., 2005, Drug Discov Today. The skilled person will know that commercial equipment is available for ΔG° measurements. ΔG° can also be estimated numerically by using the nearest neighbor model as described by SantaLucia, 1998, Proc Natl Acad Sci USA. 95: 1460-1465 using appropriately derived thermodynamic parameters described by Sugimoto et al., 1995, Biochemistry 34:11211-11216 and McTigue et al., 2004, Biochemistry 43:5388-5405. In order to have the possibility of modulating its intended nucleic acid target by hybridization, oligonucleotides of the present invention hybridize to a target nucleic acid with estimated ΔG° values below −10 kcal for oligonucleotides that are 10-30 nucleotides in length. In some embodiments the degree or strength of hybridization is measured by the standard state Gibbs free energy ΔG°. The oligonucleotides may hybridize to a target nucleic acid with estimated ΔG° values below the range of −10 kcal, such as below −15 kcal, such as below −20 kcal and such as below −25 kcal for oligonucleotides that are 8-30 nucleotides in length. In some embodiments the oligonucleotides hybridize to a target nucleic acid with an estimated ΔG° value of −10 to −60 kcal, such as −12 to −40, such as from −15 to −30 kcal or 16 to −27 kcal such as −18 to −25 kcal.

Target Sequence

The term “target sequence” as used herein refers to a sequence of nucleotides present in the target nucleic acid which comprises the nucleobase sequence which is complementary to the antisense oligonucleotide of the invention. In some embodiments, the target sequence consists of a region on the target nucleic acid with a nucleobase sequence that is complementary to the contiguous nucleotide sequence of the antisense oligonucleotide of the invention. This region of the target nucleic acid may interchangeably be referred to as the target nucleotide sequence, target sequence or target region. In some embodiments the target sequence is longer than the contiguous complementary sequence of a single oligonucleotide, and may, for example represent a preferred region of the target nucleic acid which may be targeted by several oligonucleotides of the invention.

The antisense oligonucleotide of the invention comprises a contiguous nucleotide sequence which is complementary to the target nucleic acid such as a target sequence described herein. The target sequence to which the oligonucleotide is complementary generally comprises a contiguous nucleobase sequence of at least 10 nucleotides. The contiguous nucleotide sequence is between 10 to 50 nucleotides, such as 12 to 30, such as 14 to 20, such as 15 to 18 contiguous nucleotides

Target Cell

The term “target cell” as used herein refers to a cell which is expressing the target nucleic acid. In some embodiments the target cell may be in vivo or in vitro. In some embodiments the target cell is a mammalian cell such as a rodent cell, such as a mouse cell or a rat cell, or a primate cell such as a monkey cell or a human cell.

High Affinity Modified Nucleosides

A high affinity modified nucleoside is a modified nucleotide which, when incorporated into the oligonucleotide enhances the affinity of the oligonucleotide for its complementary target, for example as measured by the melting temperature (Tₘ). A high affinity modified nucleoside of the present invention preferably result in an increase in melting temperature between +0.5 to +12° C., more preferably between +1.5 to +10° C. and most preferably between +3 to +8° C. per modified nucleoside. Numerous high affinity modified nucleosides are known in the art and include for example, many 2′ substituted nucleosides as well as locked nucleic acids (LNA) (see e.g. Freier & Altmann; Nucl. Acid Res., 1997, 25, 4429-4443 and Uhlmann; Curr. Opinion in Drug Development, 2000, 3(2), 293-213).

Sugar Modifications

The oligomer of the invention may comprise one or more nucleosides which have a modified sugar moiety, i.e. a modification of the sugar moiety when compared to the ribose sugar moiety found in DNA and RNA.

Numerous nucleosides with modification of the ribose sugar moiety have been made, primarily with the aim of improving certain properties of oligonucleotides, such as affinity and/or nuclease resistance.

Such modifications include those where the ribose ring structure is modified, e.g. by replacement with a hexose ring (HNA), or a bicyclic ring, which typically have a biradicle bridge between the C2 and C4 carbons on the ribose ring (LNA), or an unlinked ribose ring which typically lacks a bond between the C2 and C3 carbons (e.g. UNA). Other sugar modified nucleosides include, for example, bicyclohexose nucleic acids (WO2011/017521) or tricyclic nucleic acids (WO2013/154798). Modified nucleosides also include nucleosides where the sugar moiety is replaced with a non-sugar moiety, for example in the case of peptide nucleic acids (PNA), or morpholino nucleic acids.

2′ Modified Nucleosides.

A 2′ sugar modified nucleoside is a nucleoside which has a substituent other than H or —OH at the 2′ position (2′ substituted nucleoside) or comprises a 2′ linked biradicle capable of forming a bridge between the 2′ carbon and a second carbon in the ribose ring, such as LNA (2′-4′ biradicle bridged) nucleosides.

Indeed, much focus has been spent on developing 2′ substituted nucleosides, and numerous 2′ substituted nucleosides have been found to have beneficial properties when incorporated into oligonucleotides. For example, the 2′ modified sugar may provide enhanced binding affinity and/or increased nuclease resistance to the oligonucleotide. Examples of 2′ substituted modified nucleosides are 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, and 2′-F-ANA nucleoside. For further examples, please see e.g. Freier & Altmann; Nucl. Acid Res., 1997, 25, 4429-4443 and Uhlmann; Curr. Opinion in Drug Development, 2000, 3(2), 293-213, and Deleavey and Damha, Chemistry and Biology 2012, 19, 937. Below are illustrations of some 2′ substituted modified nucleosides.

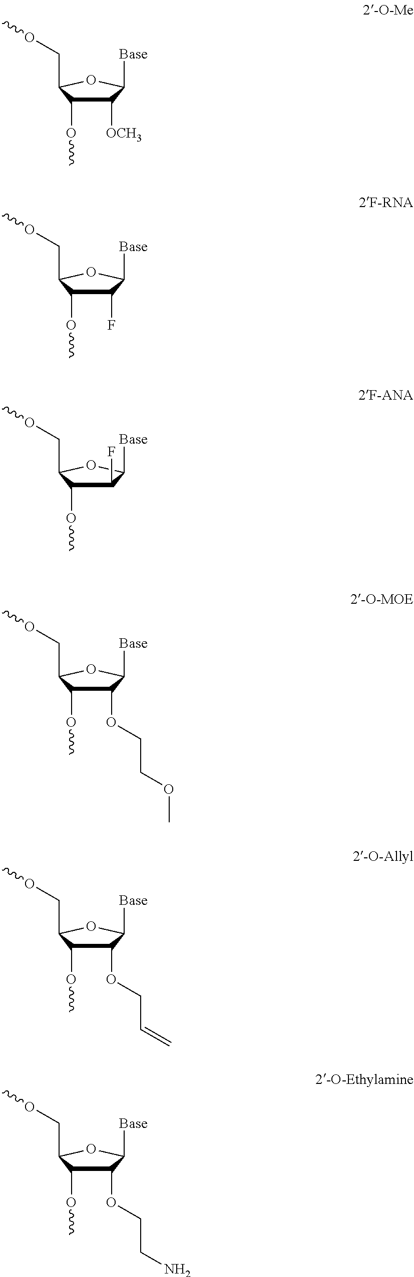

In relation to the present invention 2′ substituted sugar modified nucleosides does not include 2′ bridged nucleosides like LNA.

Locked Nucleic Acid (LNA)

A “LNA nucleoside” is a 2′-modified nucleoside which comprises a biradical linking the C2′ and C4′ of the ribose sugar ring of said nucleoside (also referred to as a “2′-4′ bridge”), which restricts or locks the conformation of the ribose ring. These nucleosides are also termed bridged nucleic acid or bicyclic nucleic acid (BNA) in the literature. The locking of the conformation of the ribose is associated with an enhanced affinity of hybridization (duplex stabilization) when the LNA is incorporated into an oligonucleotide for a complementary RNA or DNA molecule. This can be routinely determined by measuring the melting temperature of the oligonucleotide/complement duplex.

Non limiting, exemplary LNA nucleosides are disclosed in WO 99/014226, WO 00/66604, WO 98/039352, WO 2004/046160, WO 00/047599, WO 2007/134181, WO 2010/077578, WO 2010/036698, WO 2007/090071, WO 2009/006478, WO 2011/156202, WO 2008/154401, WO 2009/067647, WO 2008/150729, Morita et al., Bioorganic & Med. Chem. Lett. 12, 73-76, Seth et al. J. Org. Chem. 2010, Vol 75(5) pp. 1569-81, and Mitsuoka et al., Nucleic Acids Research 2009, 37(4), 1225-1238, and Wan and Seth, J. Medical Chemistry 2016, 59, 9645-9667.

Further non limiting, exemplary LNA nucleosides are disclosed in Scheme 1.

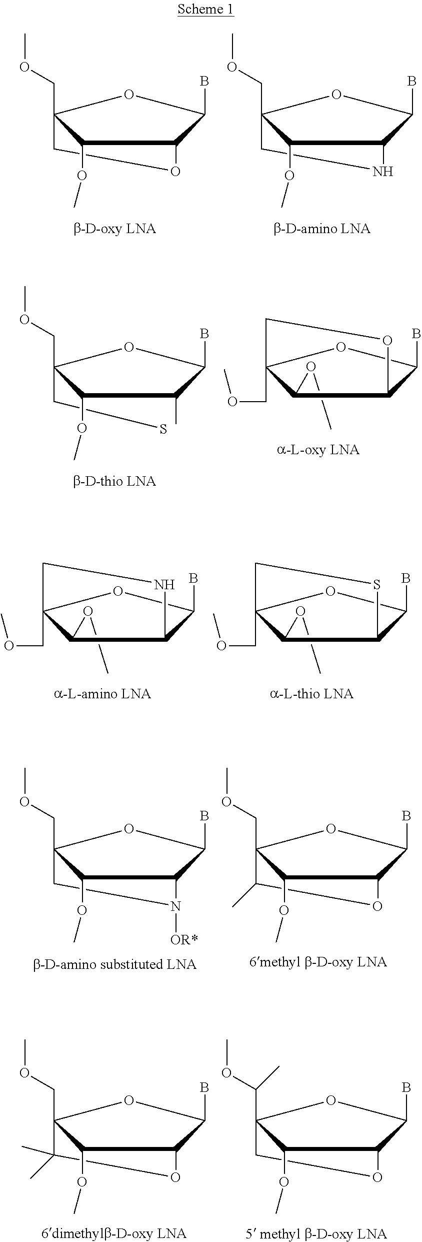

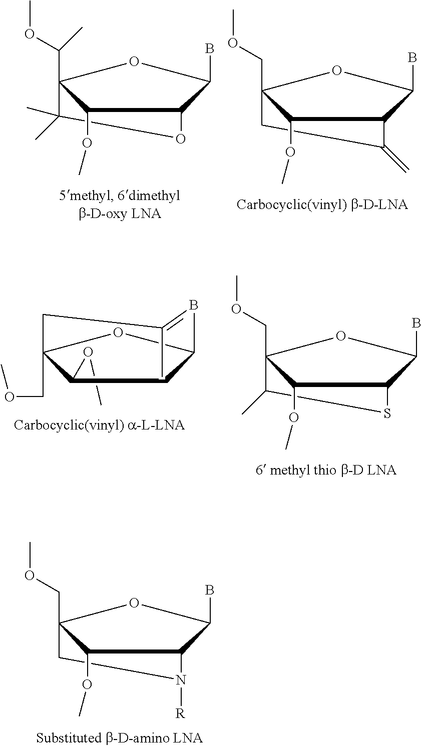

Particular LNA nucleosides are beta-D-oxy-LNA, 6′-methyl-beta-D-oxy LNA such as (S)-6′-methyl-beta-D-oxy-LNA (ScET) and ENA.

A particularly advantageous LNA is beta-D-oxy-LNA, as used in the compounds of the examples.

RNase H Activity and Recruitment

The RNase H activity of an antisense oligonucleotide refers to its ability to recruit RNase H when in a duplex with a complementary RNA molecule. WO01/23613 provides in vitro methods for determining RNaseH activity, which may be used to determine the ability to recruit RNaseH. Typically an oligonucleotide is deemed capable of recruiting RNase H if it, when provided with a complementary target nucleic acid sequence, has an initial rate, as measured in pmol/l/min, of at least 5%, such as at least 10% or more than 20% of the of the initial rate determined when using a oligonucleotide having the same base sequence as the modified oligonucleotide being tested, but containing only DNA monomers with phosphorothioate linkages between all monomers in the oligonucleotide, and using the methodology provided by Example 91-95 of WO01/23613 (hereby incorporated by reference). For use in determining RHase H activity, recombinant human RNase H1 is available from Lubio Science GmbH, Lucerne, Switzerland.

Gapmer

The antisense oligonucleotide, or contiguous nucleotide sequence thereof may be a gapmer. The antisense gapmers are commonly used to inhibit a target nucleic acid via RNase H mediated degradation. A gapmer oligonucleotide comprises at least three distinct structural regions a 5′-flank, a gap and a 3′-flank, F-G-F′ in the ‘5->3’ orientation. The “gap” region (G) comprises a stretch of contiguous DNA nucleotides which enable the oligonucleotide to recruit RNase H. The gap region is flanked by a 5′ flanking region (F) comprising one or more sugar modified nucleosides, advantageously high affinity sugar modified nucleosides, and by a 3′ flanking region (F′) comprising one or more sugar modified nucleosides, advantageously high affinity sugar modified nucleosides. The one or more sugar modified nucleosides in region F and F′ enhance the affinity of the oligonucleotide for the target nucleic acid (i.e. are affinity enhancing sugar modified nucleosides). In some embodiments, the one or more sugar modified nucleosides in region F and F′ are 2′ sugar modified nucleosides, such as high affinity 2′ sugar modifications, such as independently selected from LNA and 2′-MOE.

In a gapmer design, the 5′ and 3′ most nucleosides of the gap region are DNA nucleosides, and are positioned adjacent to a sugar modified nucleoside of the 5′ (F) or 3′ (F′) region respectively. The flanks may further defined by having at least one sugar modified nucleoside at the end most distant from the gap region, i.e. at the 5′ end of the 5′ flank and at the 3′ end of the 3′ flank.

Regions F-G-F′ form a contiguous nucleotide sequence. Antisense oligonucleotides of the invention, or the contiguous nucleotide sequence thereof, may comprise a gapmer region of formula F-G-F′.

The overall length of the gapmer design F-G-F′ may be, for example 12 to 32 nucleosides, such as 13 to 24, such as 14 to 22 nucleosides, Such as from 14 to 17, such as 16 to18 nucleosides.

By way of example, the gapmer oligonucleotide of the present invention can be represented by the following formulae:

F₁₋₈-G₅₋₁₆-F′₁₋₈, such as

F₁₋₈-G₇₋₁₆-F′₂₋₈

with the proviso that the overall length of the gapmer regions F-G-F′ is at least 12, such as at least 14 nucleotides in length.

Regions F, G and F′ are further defined below and can be incorporated into the F-G-F′ formula.

Gapmer—Region G

Region G (gap region) of the gapmer is a region of nucleosides which enables the oligonucleotide to recruit RNaseH, such as human RNase H1, typically DNA nucleosides. RNaseH is a cellular enzyme which recognizes the duplex between DNA and RNA, and enzymatically cleaves the RNA molecule. Suitably gapmers may have a gap region (G) of at least 5 or 6 contiguous DNA nucleosides, such as 5-16 contiguous DNA nucleosides, such as 6-15 contiguous DNA nucleosides, such as 7-14 contiguous DNA nucleosides, such as 8-12 contiguous DNA nucleotides, such as 8-12 contiguous DNA nucleotides in length. The gap region G may, in some embodiments consist of 6, 7, 8, 9, 10, 11, 12, 13, 14, 15 or 16 contiguous DNA nucleosides. Cytosine (C) DNA in the gap region may in some instances be methylated, such residues are either annotated as 5-methyl-cytosine (^{me}C or with an e instead of a c). Methylation of Cytosine DNA in the gap is advantageous if cg dinucleotides are present in the gap to reduce potential toxicity, the modification does not have significant impact on efficacy of the oligonucleotides.

In some embodiments the gap region G may consist of 6, 7, 8, 9, 10, 11, 12, 13, 14, 15 or 16 contiguous phosphorothioate linked DNA nucleosides. In some embodiments, all internucleoside linkages in the gap are phosphorothioate linkages.

Whilst traditional gapmers have a DNA gap region, there are numerous examples of modified nucleosides which allow for RNaseH recruitment when they are used within the gap region. Modified nucleosides which have been reported as being capable of recruiting RNaseH when included within a gap region include, for example, alpha-L-LNA, C4′ alkylated DNA (as described in PCT/EP2009/050349 and Vester et al., Bioorg. Med. Chem. Lett. 18 (2008) 2296-2300, both incorporated herein by reference), arabinose derived nucleosides like ANA and 2′F-ANA (Mangos et al. 2003 J. AM. CHEM. SOC. 125, 654-661), UNA (unlocked nucleic acid) (as described in Fluiter et al., Mol. Biosyst., 2009, 10, 1039 incorporated herein by reference). UNA is unlocked nucleic acid, typically where the bond between C2 and C3 of the ribose has been removed, forming an unlocked “sugar” residue. The modified nucleosides used in such gapmers may be nucleosides which adopt a 2′ endo (DNA like) structure when introduced into the gap region, i.e. modifications which allow for RNaseH recruitment). In some embodiments the DNA Gap region (G) described herein may optionally contain 1 to 3 sugar modified nucleosides which adopt a 2′ endo (DNA like) structure when introduced into the gap region.

Region G—“Gap-Breaker”

Alternatively, there are numerous reports of the insertion of a modified nucleoside which confers a 3′ endo conformation into the gap region of gapmers, whilst retaining some RNaseH activity. Such gapmers with a gap region comprising one or more 3′endo modified nucleosides are referred to as “gap-breaker” or “gap-disrupted” gapmers, see for example WO2013/022984. Gap-breaker oligonucleotides retain sufficient region of DNA nucleosides within the gap region to allow for RNaseH recruitment. The ability of gapbreaker oligonucleotide design to recruit RNaseH is typically sequence or even compound specific—see Rukov et al. 2015 Nucl. Acids Res. Vol. 43 pp. 8476-8487, which discloses “gapbreaker” oligonucleotides which recruit RNaseH which in some instances provide a more specific cleavage of the target RNA. Modified nucleosides used within the gap region of gap-breaker oligonucleotides may for example be modified nucleosides which confer a 3′endo confirmation, such 2′-O-methyl (OMe) or 2′-O-MOE (MOE) nucleosides, or beta-D LNA nucleosides (the bridge between C2′ and C4′ of the ribose sugar ring of a nucleoside is in the beta conformation), such as beta-D-oxy LNA or ScET nucleosides.

As with gapmers containing region G described above, the gap region of gap-breaker or gap-disrupted gapmers, have a DNA nucleosides at the 5′ end of the gap (adjacent to the 3′ nucleoside of region F), and a DNA nucleoside at the 3′ end of the gap (adjacent to the 5′ nucleoside of region F′). Gapmers which comprise a disrupted gap typically retain a region of at least 3 or 4 contiguous DNA nucleosides at either the 5′ end or 3′ end of the gap region.

Exemplary designs for gap-breaker oligonucleotides include

F₁₋₈-[D₃₋₄-E₁-D₃₋₄]-F₁₋₈

F₁₋₈-[D₁₋₄-E₁-D₃₋₄]-F′₁₋₈

F₁₋₈-[D₃₋₄-E₁-D₁₋₄]-F′₁₋₈

wherein region G is within the brackets [Dₙ-Eᵣ-Dₘ], D is a contiguous sequence of DNA nucleosides, E is a modified nucleoside (the gap-breaker or gap-disrupting nucleoside), and F and F′ are the flanking regions as defined herein, and with the proviso that the overall length of the gapmer regions F-G-F′ is at least 12, such as at least 14 nucleotides in length.

In some embodiments, region G of a gap disrupted gapmer comprises at least 6 DNA nucleosides, such as 6, 7, 8, 9, 10, 11, 12, 13, 14, 15 or 16 DNA nucleosides. As described above, the DNA nucleosides may be contiguous or may optionally be interspersed with one or more modified nucleosides, with the proviso that the gap region G is capable of mediating RNaseH recruitment.

Gapmer—Flanking Regions, F and F′

Region F is positioned immediately adjacent to the 5′ DNA nucleoside of region G. The 3′ most nucleoside of region F is a sugar modified nucleoside, such as a high affinity sugar modified nucleoside, for example a 2′ substituted nucleoside, such as a MOE nucleoside, or an LNA nucleoside.

Region F′ is positioned immediately adjacent to the 3′ DNA nucleoside of region G. The 5′ most nucleoside of region F′ is a sugar modified nucleoside, such as a high affinity sugar modified nucleoside, for example a 2′ substituted nucleoside, such as a MOE nucleoside, or an LNA nucleoside.

Region F is 1-8 contiguous nucleotides in length, such as 2-6, such as 3-4 contiguous nucleotides in length. Advantageously the 5′ most nucleoside of region F is a sugar modified nucleoside. In some embodiments the two 5′ most nucleoside of region F are sugar modified nucleoside. In some embodiments the 5′ most nucleoside of region F is an LNA nucleoside. In some embodiments the two 5′ most nucleoside of region F are LNA nucleosides. In some embodiments the two 5′ most nucleoside of region F are 2′ substituted nucleoside nucleosides, such as two 3′ MOE nucleosides. In some embodiments the 5′ most nucleoside of region F is a 2′ substituted nucleoside, such as a MOE nucleoside.

Region F′ is 2-8 contiguous nucleotides in length, such as 3-6, such as 4-5 contiguous nucleotides in length. Advantageously, embodiments the 3′ most nucleoside of region F′ is a sugar modified nucleoside. In some embodiments the two 3′ most nucleoside of region F′ are sugar modified nucleoside. In some embodiments the two 3′ most nucleoside of region F′ are LNA nucleosides. In some embodiments the 3′ most nucleoside of region F′ is an LNA nucleoside. In some embodiments the two 3′ most nucleoside of region F′ are 2′ substituted nucleoside nucleosides, such as two 3′ MOE nucleosides. In some embodiments the 3′ most nucleoside of region F′ is a 2′ substituted nucleoside, such as a MOE nucleoside.

It should be noted that when the length of region F or F′ is one, it is advantageously an LNA nucleoside.

In some embodiments, region F and F′ independently consists of or comprises a contiguous sequence of sugar modified nucleosides. In some embodiments, the sugar modified nucleosides of region F may be independently selected from 2′-O-alkyl-RNA units, 2′-O-methyl-RNA, 2′-amino-DNA units, 2′-fluoro-DNA units, 2′-alkoxy-RNA, MOE units, LNA units, arabino nucleic acid (ANA) units and 2′-fluoro-ANA units.

In some embodiments, region F and F′ independently comprises both LNA and a 2′ substituted modified nucleosides (mixed wing design).

In some embodiments, region F and F′ consists of only one type of sugar modified nucleosides, such as only MOE or only beta-D-oxy LNA or only ScET. Such designs are also termed uniform flanks or uniform gapmer design.

In some embodiments, all the nucleosides of region F or F′, or F and F′ are LNA nucleosides, such as independently selected from beta-D-oxy LNA, ENA or ScET nucleosides. In some embodiments region F consists of 1-5, such as 2-4, such as 3-4 such as 1, 2, 3, 4 or 5 contiguous LNA nucleosides. In some embodiments, all the nucleosides of region F and F′ are beta-D-oxy LNA nucleosides.

In some embodiments, all the nucleosides of region F or F′, or F and F′ are 2′ substituted nucleosides, such as OMe or MOE nucleosides. In some embodiments region F consists of 1, 2, 3, 4, 5, 6, 7, or 8 contiguous OMe or MOE nucleosides. In some embodiments only one of the flanking regions can consist of 2′ substituted nucleosides, such as OMe or MOE nucleosides. In some embodiments it is the 5′ (F) flanking region that consists 2′ substituted nucleosides, such as OMe or MOE nucleosides whereas the 3′ (F′) flanking region comprises at least one LNA nucleoside, such as beta-D-oxy LNA nucleosides or cET nucleosides. In some embodiments it is the 3′ (F′) flanking region that consists 2′ substituted nucleosides, such as OMe or MOE nucleosides whereas the 5′ (F) flanking region comprises at least one LNA nucleoside, such as beta-D-oxy LNA nucleosides or cET nucleosides.

In some embodiments, all the modified nucleosides of region F and F′ are LNA nucleosides, such as independently selected from beta-D-oxy LNA, ENA or ScET nucleosides, wherein region F or F′, or F and F′ may optionally comprise DNA nucleosides (an alternating flank, see definition of these for more details). In some embodiments, all the modified nucleosides of region F and F′ are beta-D-oxy LNA nucleosides, wherein region F or F′, or F and F′ may optionally comprise DNA nucleosides (an alternating flank, see definition of these for more details).

In some embodiments the 5′ most and the 3′ most nucleosides of region F and F′ are LNA nucleosides, such as beta-D-oxy LNA nucleosides or ScET nucleosides.

In some embodiments, the internucleoside linkage between region F and region G is a phosphorothioate internucleoside linkage. In some embodiments, the internucleoside linkage between region F′ and region G is a phosphorothioate internucleoside linkage. In some embodiments, the internucleoside linkages between the nucleosides of region F or F′, F and F′ are phosphorothioate internucleoside linkages.

Further gapmer designs are disclosed in WO 2004/046160, WO 2007/146511 and WO 2008/113832, hereby incorporated by reference.

LNA Gapmer

An LNA gapmer is a gapmer wherein either one or both of region F and F′ comprises or consists of LNA nucleosides. A beta-D-oxy gapmer is a gapmer wherein either one or both of region F and F′ comprises or consists of beta-D-oxy LNA nucleosides.

In some embodiments the LNA gapmer is of formula: [LNA]₁₋₅-[region G]-[LNA]₁₋₅, wherein region G is as defined in the Gapmer region G definition.

MOE Gapmers

A MOE gapmers is a gapmer wherein regions F and F′ consist of MOE nucleosides. In some embodiments the MOE gapmer is of design [MOE]₁₋₈-[Region G]-[MOE]₁₋₈, such as [MOE]₂₋₇-[Region G]₆₋₁₆-[MOE]₂₋₇, such as [MOE]₃₋₆-[Region G]-[MOE]₃₋₆, wherein region G is as defined in the Gapmer definition. MOE gapmers with a 5-10-5 design (MOE-DNA-MOE) have been widely used in the art.

Mixed Wing Gapmer

A mixed wing gapmer is an LNA gapmer wherein one or both of region F and F′ comprise a 2′ substituted nucleoside, such as a 2′ substituted nucleoside independently selected from the group consisting of 2′-O-alkyl-RNA units, 2′-O-methyl-RNA, 2′-amino-DNA units, 2′-fluoro-DNA units, 2′-alkoxy-RNA, MOE units, arabino nucleic acid (ANA) units and 2′-fluoro-ANA units, such as a MOE nucleosides. In some embodiments wherein at least one of region F and F′, or both region F and F′ comprise at least one LNA nucleoside, the remaining nucleosides of region F and F′ are independently selected from the group consisting of MOE and LNA. In some embodiments wherein at least one of region F and F′, or both region F and F′ comprise at least two LNA nucleosides, the remaining nucleosides of region F and F′ are independently selected from the group consisting of MOE and LNA. In some mixed wing embodiments, one or both of region F and F′ may further comprise one or more DNA nucleosides.

Mixed wing gapmer designs are disclosed in WO 2008/049085 and WO 2012/109395, both of which are hereby incorporated by reference.

Alternating Flank Gapmers

Flanking regions may comprise both LNA and DNA nucleoside and are referred to as “alternating flanks” as they comprise an alternating motif of LNA-DNA-LNA nucleosides. Gapmers comprising such alternating flanks are referred to as “alternating flank gapmers”. “Alternative flank gapmers” are thus LNA gapmer oligonucleotides where at least one of the flanks (F or F′) comprises DNA in addition to the LNA nucleoside(s). In some embodiments at least one of region F or F′, or both region F and F′, comprise both LNA nucleosides and DNA nucleosides. In such embodiments, the flanking region F or F′, or both F and F′ comprise at least three nucleosides, wherein the 5′ and 3′ most nucleosides of the F and/or F′ region are LNA nucleosides.

Alternating flank LNA gapmers are disclosed in WO 2016/127002.

Oligonucleotides with alternating flanks are LNA gapmer oligonucleotides where at least one of the flanks (F or F′) comprises DNA in addition to the LNA nucleoside(s). In some embodiments at least one of region F or F′, or both region F and F′, comprise both LNA nucleosides and DNA nucleosides. In such embodiments, the flanking region F or F′, or both F and F′ comprise at least three nucleosides, wherein the 5′ and 3′ most nucleosides of the F and/or F′ region are LNA nucleosides.

In some embodiments at least one of region F or F′, or both region F and F′, comprise both LNA nucleosides and DNA nucleosides. In such embodiments, the flanking region F or F′, or both F and F′ comprise at least three nucleosides, wherein the 5′ and 3′ most nucleosides of the F or F′ region are LNA nucleosides, and the. Flanking regions which comprise both LNA and DNA nucleoside are referred to as alternating flanks, as they comprise an alternating motif of LNA-DNA-LNA nucleosides. Alternating flank LNA gapmers are disclosed in WO2016/127002.

An alternating flank region may comprise up to 3 contiguous DNA nucleosides, such as 1 to 2 or 1 or 2 or 3 contiguous DNA nucleosides.

The alternating flak can be annotated as a series of integers, representing a number of LNA nucleosides (L) followed by a number of DNA nucleosides (D), for example

[L]₁₋₃-[D]₁₋₄-[L]₁₋₃

[L]₁₋₂-[D]₁₋₂-[L]₁₋₂-[D]₁₋₂-[L]₁₋₂

In oligonucleotide designs these will often be represented as numbers such that 2-2-1 represents 5′ [L]₂-[D]₂-[L] 3′, and 1-1-1-1-1 represents 5′ [L]-[D]-[L]-[D]-[L] 3′. The length of the flank (region F and F′) in oligonucleotides with alternating flanks may independently be 3 to 10 nucleosides, such as 4 to 8, such as 5 to 6 nucleosides, such as 4, 5, 6 or 7 modified nucleosides. In some embodiments only one of the flanks in the gapmer oligonucleotide is alternating while the other is constituted of LNA nucleotides. It may be advantageous to have at least two LNA nucleosides at the 3′ end of the 3′ flank (F′), to confer additional exonuclease resistance. Some examples of oligonucleotides with alternating flanks are:

[L]₁₋₅-[D]₁₋₄-[L]₁₋₃-[G]₅₋₁₆-[L]₂₋₆

[L]₁₋₂-[D]₁₋₂-[L]₁₋₂-[D]₁₋₂-[L]₁₋₂-[G]₅₋₁₆-[L]₁₋₂-[D]₁₋₃-[L]₂₋₄

[L]₁₋₅-[G]₅₋₁₆-[L]-[D]-[L]-[D]-[L]₂

with the proviso that the overall length of the gapmer is at least 12, such as at least 14 nucleotides in length.

Region D′ or D″ in an Oligonucleotide

The oligonucleotide of the invention may in some embodiments comprise or consist of the contiguous nucleotide sequence of the oligonucleotide which is complementary to the target nucleic acid, such as the gapmer F-G-F′, and further 5′ and/or 3′ nucleosides. The further 5′ and/or 3′ nucleosides may or may not be fully complementary to the target nucleic acid. Such further 5′ and/or 3′ nucleosides may be referred to as region D′ and D″ herein.

The addition of region D′ or D″ may be used for the purpose of joining the contiguous nucleotide sequence, such as the gapmer, to a conjugate moiety or another functional group. When used for joining the contiguous nucleotide sequence with a conjugate moiety is can serve as a biocleavable linker. Alternatively it may be used to provide exonuclease protection or for ease of synthesis or manufacture.

Region D′ and D″ can be attached to the 5′ end of region F or the 3′ end of region F′, respectively to generate designs of the following formulas D′-F-G-F′, F-G-F′-D″ or

D′-F-G-F′-D″. In this instance the F-G-F′ is the gapmer portion of the oligonucleotide and region D′ or D″ constitute a separate part of the oligonucleotide.

Region D′ or D″ may independently comprise or consist of 1, 2, 3, 4 or 5 additional nucleotides, which may be complementary or non-complementary to the target nucleic acid. The nucleotide adjacent to the F or F′ region is not a sugar-modified nucleotide, such as a DNA or RNA or base modified versions of these. The D′ or D′ region may serve as a nuclease susceptible biocleavable linker (see definition of linkers). In some embodiments the additional 5′ and/or 3′ end nucleotides are linked with phosphodiester linkages, and are DNA or RNA. Nucleotide based biocleavable linkers suitable for use as region D′ or D″ are disclosed in WO 2014/076195, which include by way of example a phosphodiester linked DNA dinucleotide. The use of biocleavable linkers in poly-oligonucleotide constructs is disclosed in WO 2015/113922, where they are used to link multiple antisense constructs (e.g. gapmer regions) within a single oligonucleotide.

In one embodiment the oligonucleotide of the invention comprises a region D′ and/or D″ in addition to the contiguous nucleotide sequence which constitutes the gapmer.

In some embodiments, the oligonucleotide of the present invention can be represented by the following formulae:

F-G-F′; in particular F₁₋₈-G₅₋₁₆-F′₂₋₈

D′-F-G-F′, in particular D′₁₋₃-F₁₋₈-G₅₋₁₆-F′₂₋₈

F-G-F′-D″, in particular F₁₋₈-G₅₋₁₆-F′₂₋₈-D″₁₋₃

D′-F-G-F′-D″, in particular D′₁₋₃-F₁₋₈-G₅₋₁₆-F′₂₋₈-D″₁₋₃

In some embodiments the internucleoside linkage positioned between region D′ and region F is a phosphodiester linkage. In some embodiments the internucleoside linkage positioned between region F′ and region D″ is a phosphodiester linkage.

Conjugate

The term conjugate as used herein refers to an oligonucleotide which is covalently linked to a non-nucleotide moiety (conjugate moiety or region C or third region).

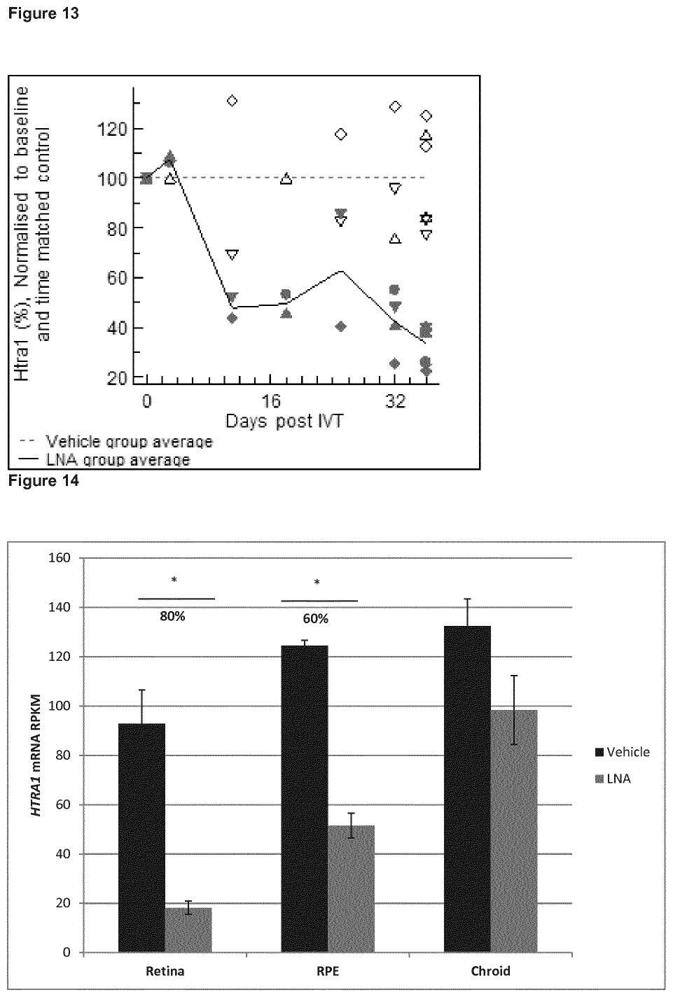

Conjugation of the oligonucleotide of the invention to one or more non-nucleotide moieties may improve the pharmacology of the oligonucleotide, e.g. by affecting the activity, cellular distribution, cellular uptake or stability of the oligonucleotide. In some embodiments the conjugate moiety modify or enhance the pharmacokinetic properties of the oligonucleotide by improving cellular distribution, bioavailability, metabolism, excretion, permeability, and/or cellular uptake of the oligonucleotide. In particular the conjugate may target the oligonucleotide to a specific organ, tissue or cell type and thereby enhance the effectiveness of the oligonucleotide in that organ, tissue or cell type. A the same time the conjugate may serve to reduce activity of the oligonucleotide in non-target cell types, tissues or organs, e.g. off target activity or activity in non-target cell types, tissues or organs. WO 93/07883 and WO2013/033230 provides suitable conjugate moieties, which are hereby incorporated by reference. Further suitable conjugate moieties are those capable of binding to the asialoglycoprotein receptor (ASGPr). In particular tri-valent N-acetylgalactosamine conjugate moieties are suitable for binding to the ASGPr, see for example WO 2014/076196, WO 2014/207232 and WO 2014/179620 (hereby incorporated by reference, in particular, FIG. 13 of WO2014/076196 or claims 158-164 of WO2014/179620).

Oligonucleotide conjugates and their synthesis has also been reported in comprehensive reviews by Manoharan in Antisense Drug Technology, Principles, Strategies, and Applications, S. T. Crooke, ed., Ch. 16, Marcel Dekker, Inc., 2001 and Manoharan, Antisense and Nucleic Acid Drug Development, 2002, 12, 103, each of which is incorporated herein by reference in its entirety.

In an embodiment, the non-nucleotide moiety (conjugate moiety) is selected from the group consisting of carbohydrates, cell surface receptor ligands, drug substances, hormones, lipophilic substances, polymers, proteins, peptides, toxins (e.g. bacterial toxins), vitamins, viral proteins (e.g. capsids) or combinations thereof.

Linkers

A linkage or linker is a connection between two atoms that links one chemical group or segment of interest to another chemical group or segment of interest via one or more covalent bonds. Conjugate moieties can be attached to the oligonucleotide directly or through a linking moiety (e.g. linker or tether). Linkers serve to covalently connect a third region, e.g. a conjugate moiety (Region C), to a first region, e.g. an oligonucleotide or contiguous nucleotide sequence complementary to the target nucleic acid (region A), thereby connecting one of the termini of region A to C.

In some embodiments of the invention the conjugate or oligonucleotide conjugate of the invention may optionally, comprise a linker region (second region or region B and/or region Y) which is positioned between the oligonucleotide or contiguous nucleotide sequence complementary to the target nucleic acid (region A or first region) and the conjugate moiety (region C or third region).

Region B refers to biocleavable linkers comprising or consisting of a physiologically labile bond that is cleavable under conditions normally encountered or analogous to those encountered within a mammalian body. Conditions under which physiologically labile linkers undergo chemical transformation (e.g., cleavage) include chemical conditions such as pH, temperature, oxidative or reductive conditions or agents, and salt concentration found in or analogous to those encountered in mammalian cells. Mammalian intracellular conditions also include the presence of enzymatic activity normally present in a mammalian cell such as from proteolytic enzymes or hydrolytic enzymes or nucleases. In one embodiment the biocleavable linker is susceptible to S1 nuclease cleavage. In a preferred embodiment the nuclease susceptible linker comprises between 1 and 10 nucleosides, such as 1, 2, 3, 4, 5, 6, 7, 8, 9 or 10 nucleosides, more preferably between 2 and 6 nucleosides and most preferably between 2 and 4 linked nucleosides comprising at least two consecutive phosphodiester linkages, such as at least 3 or 4 or 5 consecutive phosphodiester linkages. Preferably the nucleosides are DNA or RNA. Phosphodiester containing biocleavable linkers are described in more detail in WO 2014/076195 (hereby incorporated by reference).

Conjugates may also be linked to the oligonucleotide via non-biocleavable linkers, or in some embodiments the conjugate may comprise a non-cleavable linker which is covalently attached to the biocleavable linker (region Y). Linkers that are not necessarily biocleavable but primarily serve to covalently connect a conjugate moiety (region C or third region), to an oligonucleotide (region A or first region), may comprise a chain structure or an oligomer of repeating units such as ethylene glycol, amino acid units or amino alkyl groups The oligonucleotide conjugates of the present invention can be constructed of the following regional elements A-C, A-B-C, A-B-Y-C, A-Y-B-C or A-Y-C. In some embodiments the non-cleavable linker (region Y) is an amino alkyl, such as a C2-C36 amino alkyl group, including, for example C6 to C12 amino alkyl groups. In a preferred embodiment the linker (region Y) is a C6 amino alkyl group. Conjugate linker groups may be routinely attached to an oligonucleotide via use of an amino modified oligonucleotide, and an activated ester group on the conjugate group.

HTRA1

The term “HTRA1” as used herein, refers to any native refers to a mammalian such as a primate or human high temperature requirement A1 protein from any mammalian source, including primates (e.g., humans) and rodents (e.g., mice and rats), unless otherwise indicated. The term encompasses “full-length,” unprocessed HTRA1 as well as any form of HTRA1 that result from processing in the cell. The term also encompasses naturally occurring variants of HTRA1, e.g., splice variants or allelic variants. The UniProt Accession number for human HTRA1 is Q92743. The amino acid sequence of an exemplary human HTRA1 is shown in SEQ ID NO: 1.

SEQ ID NO 1:
MQIPRAALLPLLLLLLAAPASAQLSRAGRSAPLAAGCPDRCEP
ARCPPQPEHCEGGRARDACGCCEVCGAPEGAACGLQEGPCGEG
LQCVVPFGVPASATVRRRAQAGLCVCASSEPVCGSDANTYANL
CQLRAASRRSERLHRPPVIVLQRGACGQGQEDPNSLRHKYNFI
ADVVEKIAPAVVHIELFRKLPFSKREVPVASGSGFIVSEDGLI
VTNAHVVTNKHRVKVELKNGATYEAKIKDVDEKADIALIKIDH
QGKLPVLLLGRSSELRPGEFVVAIGSPFSLQNTVTTGIVSTTQ
RGGKELGLRNSDMDYIQTDAIINYGNSGGPLVNLDGEVIGINT
LKVTAGISFAIPSDKIKKFLTESHDRQAKGKAITKKKYIGIRM
MSLTSSKAKELKDRHRDFPDVISGAYIIEVIPDTPAEAGGLKE
NDVIISINGQSVVSANDVSDVIKRESTLNMVVRRGNEDIMITV
IPEEIDP

The amino acid sequence of an exemplary primate HTRA1 is shown in SEQ ID NO: 2 (Macaca fascicularis). A Macaca mulatta HTRA1 amino acis sequence is disclosed in Uniprot Accession number H9FX91).

SEQ ID NO 2
MQIPRAALLPLLLLLLLAAPASAQLSRAGRSPEHCEGGRARDA
CGCCEVCGAPEGAACGLQEGPCGEGLQCVVPFGVPASATVRRR
AQAGLCVCASNEPVCGSDANTYANLCQLRAASRRSERLHRPPV
IVLQRGACGQGQEDPNSLRHKYNFIADVVEKIAPAVVHIELFR
KLPFSKREVPVASGSGFIVSEDGLIVTNAHVVTNKHRVKVELK
NGATYEAKIKDVDEKADIALIKIDHQGKLPVLLLGRSSELRPG
EFVVAIGSPFSLQNTVTTGIVSTTQRGGKELGLRNSDMDYIQT
DAIINYGNSGGPLVNLDGEVIGINTLKVTAGISFAIPSDKIKK
FLTESHDRQAKGKAITKKKYIGIRMMSLTSSKAKELKDRHRDF
PDVISGAYIIEVIPDTPAEAGGLKENDVIISINGQSVVSANDV
SDVIKRESTLNMVVRRGNEDIMITVIPEEIDP

HTRA1 is also known in the art as protease, serine, 11 (IGF binding) (PRSS11), ARMD7, HtrA, and IGFBP5-protease. The term “HTRA1” also encompasses “HTRA1 variants,” which means an active HtrA1 polypeptide having at least about 90% amino acid sequence identity to a native sequence HtrA1 polypeptide, such as SEQ ID NO: 1 or SEQ ID NO 2. Ordinarily, a HTRA1 variant will have at least about 95% amino acid sequence identity, or at least about 98% amino acid sequence identity, or at least about 99% amino acid sequence identity with a native HTRA1 sequence, e.g., SEQ ID NO: 1 or SEQ ID NO 2.

The term “HTRA1 mRNA antagonist” is used to refer to an HTRA1 antagonist which targets a HTRA1 nucleic acid, such as a mRNA, including the HTRA1 mRNA or pre-mRNA. In some embodiments the HTRA1 mRNA antagonist is an antisense oligonucleotide or a siRNA and shRNA or a ribozyme.

An HTRA1 mRNA antagonist is capable of inhibiting the expression of the HTRA1 target nucleic acid in a cell which is expressing the HTRA1 target nucleic acid. Advantageously the HTRA1 mRNA antagonist is or comprises an oligonucleotide where the contiguous sequence of nucleobases of the oligonucleotide is complementary to, such as fully complementary to, the HTRA1 target nucleic acid, as measured across the length of the oligonucleotide or contiguous nucleotide sequence thereof. The HTRA1 target nucleic acid may, in some embodiments, be a RNA or DNA, such as a messenger RNA, such as a mature mRNA or a pre-mRNA. In some embodiments the target nucleic acid is a RNA which encodes mammalian HTRA1 protein, such as human HTRA1, e.g. the human HTRA1 mRNA sequence, such as that disclosed as SEQ ID NO 3 or 4. Further information on exemplary target nucleic acids is provided in tables 1 & 2.

TABLE 1
Genome and assembly information for human and Cyno HTRA1.

| | | | | | | NCBI |
|---|---|---|---|---|---|---|
| | | | | | | reference |
| | | | | | | sequence* |
| | | | | | | accession |
| | | | Genomic coordinates | | | number |
| Species | Chr. | Strand | Start | End | Assembly | for mRNA |
| Human | 10 | fwd | 122461525 | 122514908 | GRCh38.p2 release 107 | NM_002775.4 |
| Cyno | 9 | fwd | 121764994 | 121817518 | Macaca_fascicularis_5.0 | NC_022280.1** |

Fwd = forward strand.
The genome coordinates provide the pre-mRNA sequence (genomic sequence). The NCBI reference provides the mRNA sequence (cDNA sequence).
*The National Center for Biotechnology Information reference sequence database is a comprehensive, integrated, non-redundant, well-annotated set of reference sequences including genomic, transcript, and protein. It is hosted at www.ncbi.nlm.nih.gov/refseq.
**In the NCBI reference sequence there is a stretch of 100 nucleotides from position 126 to position 227 whose identity is not known. In SEQ ID NO 5 & 6, this stretch has been replaced by the nucleotides appearing in both human and Macaca mulatta HTRA1 premRNA sequences in this region.

TABLE 2
Sequence details for human and Cyno HTRA1.

| | | Length | SEQ ID |
|---|---|---|---|
| Species | RNA type | (nt) | NO |
| Human | mRNA | 2138 | 3 |
| Human | premRNA | 53384 | 4 |
| Cyno | mRNA | 2123 | 5 |
| Cyno | premRNA | 52575 | 6 |

Oligonucleotide Antagonists of HTRA1

PCT/EP2017/065937 and EP17173964.2, both of which are incorporated by reference in their entirety, disclose numerous antisense oligonucleotides which are potent in vivo inhibitors of HTRA1 mRNA and their therapeutic use, including use to treat macular degeneration, which may be used in the present invention.

The HTRA1 mRNA antagonists may be antisense oligonucleotides or siRNAs which comprise a contiguous nucleotide sequence of 10-30 nucleotides in length with at least 90% complementarity, such as fully complementary, to a mammalian HTRA1 nucleic acid, such as SEQ ID NO 3, SEQ ID NO 4, SEQ ID NO 5 or SEQ ID NO 6.

Advantageously, the HTRA1 mRNA antagonist is an LNA antisense oligonucleotide, such as an LNA gapmer oligonucleotide.

In some embodiments, the HTRA1 mRNA antagonists, such as the antisense oligonucleotide, including the LNA antisense oligonucleotide or gapmer oligonucleotide, comprises a contiguous nucleotide region of 10-22 nucleotides which are at least 90% such as 100% complementarity to SEQ ID NO 7:

SEQ ID NO 7:
5′ CCAACAACCAGGTAAATATTTG 3′

In some embodiments, the HTRA1 mRNA antagonists, such as the antisense oligonucleotide or siRNA; such as an LNA antisense oligonucleotide or gapmer oligonucleotide, comprises a contiguous nucleotide region of at least 12 contiguous nucleotides in length present in a sequence selected from the group consisting of

SEQ ID NO 8:
CAAATATTTACCTGGTTG
SEQ ID NO 9:
TTTACCTGGTTGTTGG
SEQ ID NO 10:
CCAAATATTTACCTGGTT
SEQ ID NO 11:
CCAAATATTTACCTGGTTGT
SEQ ID NO 12:
ATATTTACCTGGTTGTTG
SEQ ID NO 13:
TATTTACCTGGTTGTT
SEQ ID NO 14:
ATATTTACCTGGTTGT,
and
SEQ ID NO 15:
ATATTTACCTGGTTGTT.

In some embodiments, the HTRA1 mRNA antagonist (antisense oligonucleotide) is or comprises an oligonucleotide selected from the group selected from:

(SEQ ID NO 8, Comp # 8,1)
CₛAₛAₛAₛtₛaₛtₛtₛtₛaₛcₛcₛtₛgₛGₛTₛTₛG
(SEQ ID NO 9, Comp # 9,1)
TₛTₛtₛaₛcₛcₛtₛgₛgₛtₛtₛgₛtₛTₛGₛG
(SEQ ID NO 10, Comp # 10,1)
CₛCₛAₛAₛaₛtₛaₛtₛtₛtₛaₛcₛcₛtₛgₛGₛTₛT
(SEQ ID NO 11, Comp # 11,1)
CₛCₛAₛaₛaₛtₛaₛtₛtₛtₛaₛcₛcₛtₛgₛgₛtₛtₛGₛT
(SEQ ID NO 12, Comp # 12,1)
AₛTₛAₛtₛtₛtₛaₛcₛcₛtₛgₛgₛtₛtₛgₛTₛTₛG
(SEQ ID NO 13, Comp # 13,1)
TₛAₛTₛtₛtₛaₛcₛcₛtₛgₛgₛtₛTₛGₛTₛT
(SEQ ID NO 13, Comp # 13,2)
TₛAₛtₛtₛtₛaₛcₛcₛtₛgₛGₛtₛTₛgₛTₛT
(SEQ ID NO 13, Comp # 13,3)
TₛAₛTₛtₛtₛaₛcₛcₛtₛgₛgₛTₛTₛgₛTₛT
(SEQ ID NO 14, Comp # 14,1)
AₛTₛAₛTₛtₛtₛaₛcₛcₛtₛgₛgₛTₛTₛGₛT
(SEQ ID NO 14, Comp # 14,2)
AₛtₛAₛTₛTₛtₛaₛcₛcₛtₛgₛgₛtₛTₛGₛT
(SEQ ID NO 15, Comp # 15,1)
AₛtₛAₛTₛtₛtₛaₛcₛcₛtₛgₛGₛTₛTₛgₛTₛT
(SEQ ID NO 15, Comp # 15,2)
AₛTₛAₛtₛtₛtₛaₛcₛcₛtₛgₛGₛtₛTₛgₛTₛT
(SEQ ID NO 15, Comp # 15,3)
AₛtₛAₛTₛtₛtₛaₛcₛcₛtₛgₛgₛtₛTₛGₛTₛT

Wherein a capital letter represents a beta-D oxy LNA nucleoside unit, a lower case letter represents a DNA nucleoside unit, subscript s represents a phosphorothioate internucleoside linkage, wherein all LNA cytosines are 5-methyl cytosine.

In some embodiments the HTRA1 mRNA antagonist is or comprises the LNA antisense oligonucleotide of formula 5′ TₛAₛTₛtₛtₛaₛcₛcₛtₛgₛgₛtₛTₛGₛTₛT 3′ (SEQ ID NO 13) or 5′ AₛtₛAₛTₛtₛtₛaₛcₛcₛtₛgₛgₛtₛTₛGₛTₛT 3′ (SEQ ID NO 15) wherein a capital letter represents an beta-D oxy LNA nucleoside unit, a lower case letter represents a DNA nucleoside unit, subscript s represents a phosphorothioate internucleoside linkage, wherein all LNA cytosines are 5-methyl cytosine, or a pharmaceutically acceptable salt thereof.

In some embodiments, the antisense oligonucleotide is of 10-30 nucleotides in length, wherein said antisense oligonucleotide targets a HTRA1 nucleic acid, and comprises a contiguous nucleotide region of 10-22 nucleotides which are at least 90% such as 100% complementarity to SEQ ID NO 16.

SEQ ID NO 16:
GACAGTCAGCATTTGTCTCCTCCTTTAACTGAGTCATCATCTT
AGTCCAACTAATGCAGTCGATACAATGCGTAGATAGAAGAAGC
CCCACGGGAGCCAGGATGGGACTGGTCGTGTTTGTGCTTTTCT
CCAAGTCAGCACCCAAAGGTCAATGCACAGAGACCCCGGGTGG
GTGAGCGCTGGCTTCTCAAACGGCCGAAGTTGCCTCTTTTAGG
AATCTCTTTGGAATTGGGAGCACGATGACTCTGAGTTTGAGCT
ATTAAAGTACTTCTTAC

In some embodiments, the antisense oligonucleotide is or comprises a contiguous nucleotide region selected from any one of SEQ ID NO 17, 18 and 19, or at least 12 contiguous nucleotides thereof:

(SEQ ID NO 17)
CTTCTTCTATCTACGCAT
(SEQ ID NO 18)
TACTTTAATAGCTCAA
(SEQ ID NO 19)
TTCTATCTACGCATTG

In some embodiments, the antisense oligonucleotide is or comprises a contiguous nucleotide region selected from:

(SEQ ID NO 17)
CTTCttctatctacgcAT,
(SEQ ID NO 18)
TACTttaatagcTCAA,
and
(SEQ ID NO 19)
TTCtatctacgcaTTG;

wherein capital letters are LNA nucleotides, and lower case letters are DNA nucleosides, and cytosine residues are optionally 5-methyl cytosine.

In some embodiments, the antisense oligonucleotide is or comprises a contiguous nucleotide region selected from:

(SEQ ID NO 17, C ID#17)
^{m}CₛTₛTₛ^{m}Cₛtₛtₛcₛtₛaₛtₛcₛtₛaₛ^{m}cₛgₛcₛAₛT,
(SEQ ID NO 18, C ID#18)
TₛAₛ^{m}CₛTₛtₛtₛaₛaₛtₛaₛgₛcₛTₛ^{m}CₛAₛA,
(SEQ ID NO 19, C ID#19),
TₛTₛ^{m}Cₛtₛaₛtₛcₛtₛaₛ^{m}cₛgₛcₛaₛTₛTₛG,

wherein capital letters represent beta-D-oxy LNA nucleosides, lower case letters are DNA nucleosides, subscript s represents a phosphorothioate internucleoside linkage, and ^{m}C represent 5 methyl cytosine beta-D-oxy LNA nucleosides, and ^{m}c represents 5 methyl cytosine DNA nucleosides.

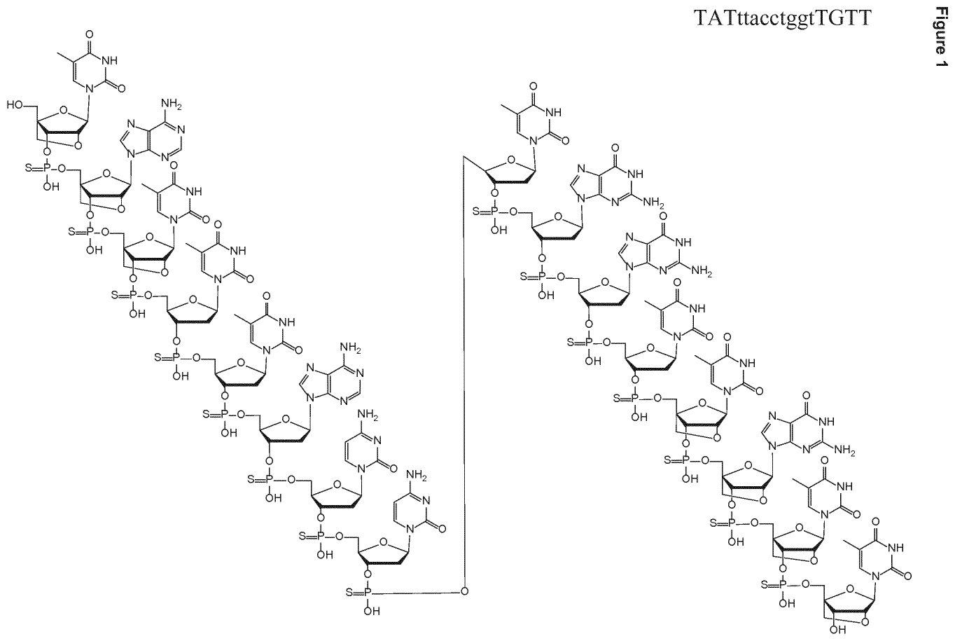

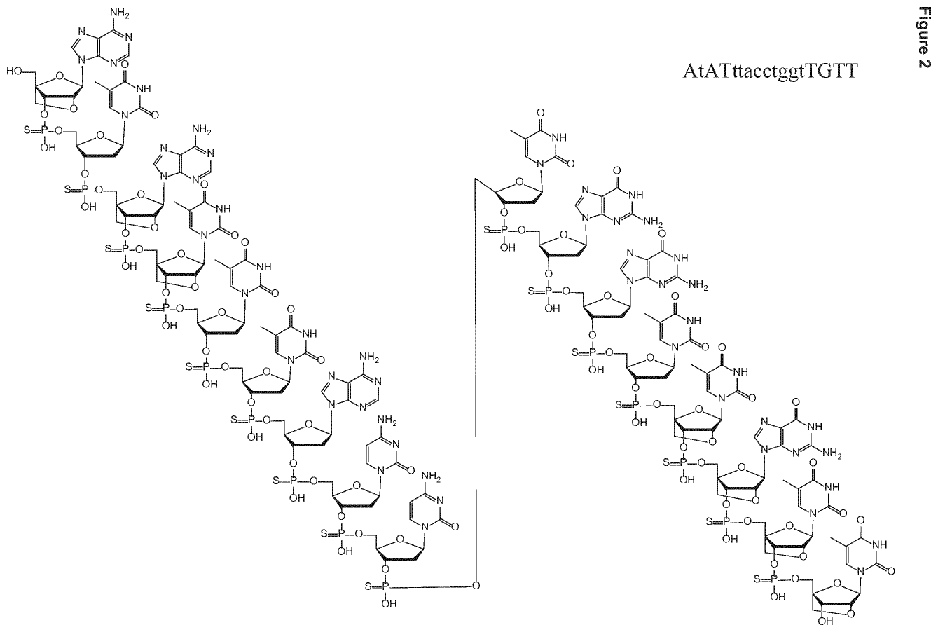

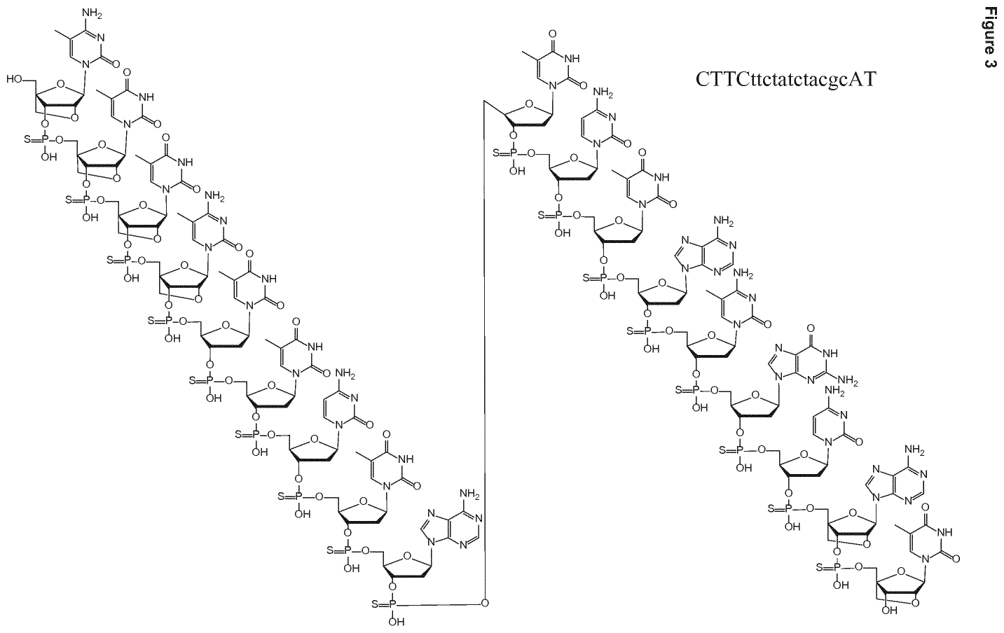

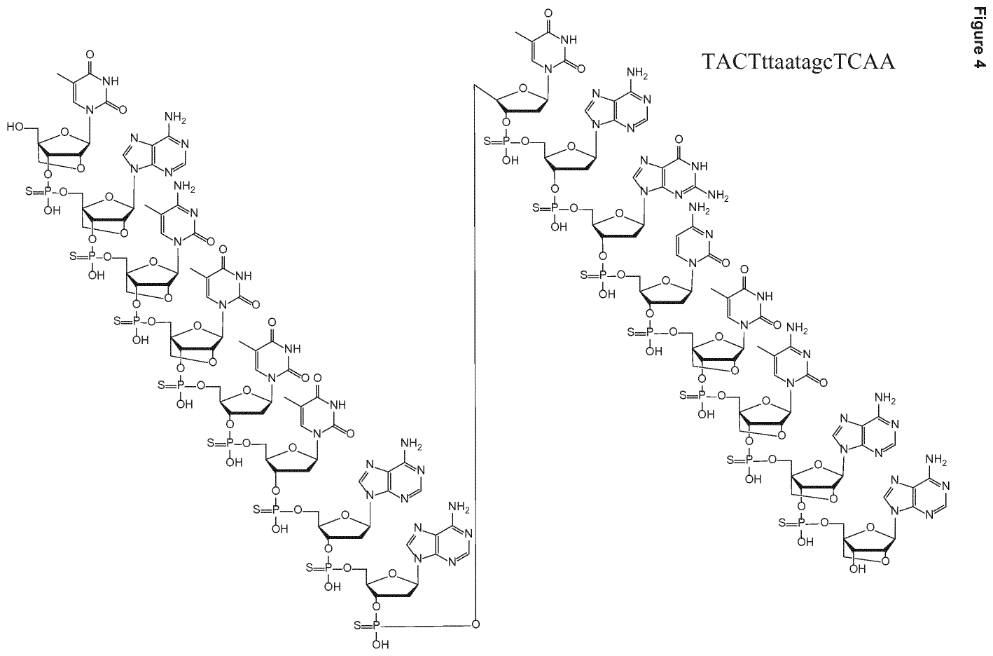

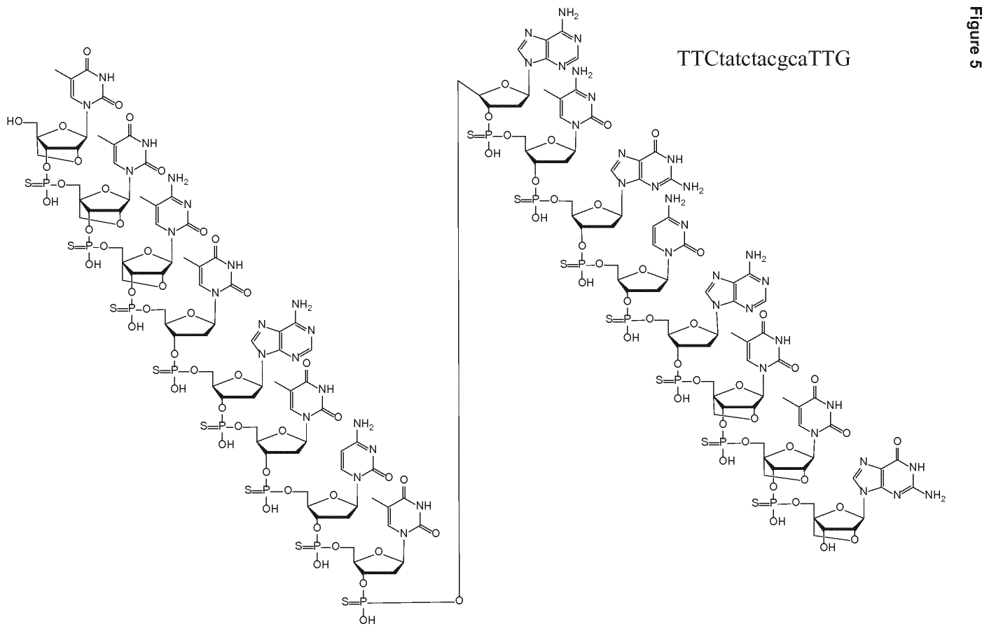

See FIGS. 1, 2, 3, 4 and 5 for exemplary LNA antisense oligonucleotide compounds which are HTRA1 mRNA antagonists for use according to the present invention or in the methods according to the present invention.

Ocular and HTRA1 Associated Disorders

The term “HtrA1-associated disorder,” as used herein, refers in the broadest sense to any disorder or condition associated with HtrA1 expression or activities, including abnormal HTRA1 expression or activities. In some embodiments, HTRA1-associated disorders are associated with excess HTRA1 levels or activity in which atypical symptoms may manifest due to the levels or activity of HTRA1 locally (e.g., in an eye) and/or systemically in the body. Exemplary HTRA1-associated disorders include HTRA1-associated ocular disorders, which include, but are not limited to, for example, age-related macular degeneration (AMD), including wet (exudative) AMD (including early, intermediate, and advanced wet AMD) and dry (nonexudative) AMD (including early, intermediate, and advanced dry AMD (e.g., geographic atrophy (GA)).

As used herein, the term “ocular disorder” includes, but is not limited to, disorders of the eye including macular degenerative diseases such as age-related macular degeneration (AMD), including wet (exudative) AMD (including early, intermediate, and advanced wet AMD) and dry (nonexudative) AMD (including early, intermediate, and advanced dry AMD (e.g., geographic atrophy (GA)); diabetic retinopathy (DR) and other ischemia-related retinopathies; endophthalmitis; uveitis; choroidal neovascularization (CNV); retinopathy of prematurity (ROP); polypoidal choroidal vasculopathy (PCV); diabetic macular edema; pathological myopia; von Hippel-Lindau disease; histoplasmosis of the eye; Central Retinal Vein Occlusion (CRVO); corneal neovascularization; and retinal neovascularization. In some embodiments, the ocular disorder is AMD (e.g., GA).

The Subject

An “individual” or “subject” is a mammal. Mammals include, primates (e.g., humans and non-human primates such as monkeys), and rodents (e.g., mice and rats). In certain embodiments, the individual or subject is a human. A “subject” may be a “patient”—a patient is a human subject who is in need of treatment, and may be an individual who has an HTRA1 related disorder, such as an ocular disorder, a subject who is at risk of developing an HTRA1 related disorder, such as an ocular disorder.

In some embodiments, the patient is a person who has been diagnosed with an ocular disorder, such as those listed herein, such as an ocular disorder selected from the group consisting of AMD, diabetic retinopathy, retinopathy of prematurity, or polypoidal choroidal vasculopathy. In some embodiments the ocular disorder is selected from the group consisting of early dry AMD, intermediate dry AMD, or advanced dry AMD. In some embodiments the ocular disorder is geographic atrophy.

In some embodiments, the patient is a person who has been identified at being of risk of developing an ocular disorder such as those listed herein, such as an ocular disorder selected from the group consisting of AMD, diabetic retinopathy, retinopathy of prematurity, or polypoidal choroidal vasculopathy. In some embodiments the ocular disorder is selected from the group consisting of early dry AMD, intermediate dry AMD, or advanced dry AMD. In some embodiments the ocular disorder is geographic atrophy.

In some embodiments, the patient is a person who had elevated HTRA1 levels in their aqueous or vitreous humor. In some embodiments, the patient is a person who has been diagnosed with an HTRA1 associated disorder or a person who has been identified at being of risk of developing an HTRA1 associated disorder. The patient may therefore be a subject, who has elevated HTRA1 levels in their aqueous or vitreous humor, and optionally may be asymptomatic.

In some embodiments, the patient may be a subject who has one or more disease associated polymorphisms in the HTRA1 gene or HTRA1 control sequence, such as the HTRA1 promoter polymorphism rs11200638(G/A) (see e.g., DeWan et al., Science 314: 989-992, 2006, which is incorporated herein by reference in its entirety). The patient may therefore be a subject, who has a disease associated polymorphism in their HTRA1 gene or HTRA1 control sequence and optionally may be asymptomatic.

Pharmaceutical Salts

For use as a therapeutic, the HTRA1 mRNA antagonist, such as an oligonucleotide targeting HTRA1, used according to the method or use of the invention, may be provided as a suitable pharmaceutical salt, such as a sodium or potassium salt. In some embodiments the oligonucleotide of the invention is a sodium salt.

Pharmaceutical Composition

In a further aspect, the invention provides pharmaceutical compositions comprising any of the aforementioned oligonucleotides and/or oligonucleotide conjugates and a pharmaceutically acceptable diluent, carrier, salt and/or adjuvant. A pharmaceutically acceptable diluent includes phosphate-buffered saline (PBS) and pharmaceutically acceptable salts include, but are not limited to, sodium and potassium salts. In some embodiments the pharmaceutically acceptable diluent is sterile phosphate buffered saline. In some embodiments the oligonucleotide is used in the pharmaceutically acceptable diluent at a concentration of 50-300 μM solution. In some embodiments, the oligonucleotide of the invention is administered at a dose of 10-1000 μg.

WO 2007/031091 provides suitable and preferred examples of pharmaceutically acceptable diluents, carriers and adjuvants (hereby incorporated by reference). Suitable dosages, formulations, administration routes, compositions, dosage forms, combinations with other therapeutic agents, pro-drug formulations are also provided in WO2007/031091. Oligonucleotides or oligonucleotide conjugates of the invention may be mixed with pharmaceutically acceptable active or inert substances for the preparation of pharmaceutical compositions or formulations. Compositions and methods for the formulation of pharmaceutical compositions are dependent upon a number of criteria, including, but not limited to, route of administration, extent of disease, or dose to be administered.

In some embodiments, the oligonucleotide or oligonucleotide conjugate of the invention is a prodrug. In particular with respect to oligonucleotide conjugates the conjugate moiety is cleaved of the oligonucleotide once the prodrug is delivered to the site of action, e.g. the target cell.

Administration

HTRA1 mRNA antagonists, may be administered via topical (such as, to the skin, inhalation, ophthalmic or otic) or enteral (such as, orally or through the gastrointestinal tract) or parenteral (such as, intravenous, subcutaneous, intra-muscular, intracerebral, intracerebroventricular or intrathecal).

In some embodiments the as HTRA1 mRNA antagonists, are administered by a parenteral route including intravenous, intraarterial, subcutaneous, intraperitoneal or intramuscular injection or infusion, intrathecal or intracranial, e.g. intracerebral or intraventricular, administration. In some embodiments the active oligonucleotide or oligonucleotide conjugate is administered intravenously. In another embodiment the active oligonucleotide or oligonucleotide conjugate is administered subcutaneously.

For use in treating occular disorders, HTRA1 mRNA antagonists, for example the LNA oligonucleotides described herein, intraocular injection may be used.

In some embodiments, the compound of the invention, or pharmaceutically acceptable salt thereof, is administered via an intraocular injection in a dose from about 10 μg to about 200 μg per eye, such as about 50 μg to about 150 μg per eye, such as about 100 μg per eye. In some embodiments, the dosage interval, i.e. the period of time between consecutive dosings is at least monthly, such as at least bi monthly or at least once every three months.

Determining the Level of HTRA1 in a Sample

A sample of the vitreous or aqueous humor, advantageously the aqueous humor, is obtained from the subject. The level of HTRA1 present in the sample may be determined by any known method in the art, suitably via the use of an anti-HTRA1 antibody in an immuno-assay, or via the use of mass spectroscopy.

In the some embodiments, HTRA1 protein levels are determined using mass-spectrometry.

In some embodiments, the level of HTRA1 protein is determined using an immuno assay, such as using an HTRA1 specific antibody.

In some embodiments, the HTRA1 protein levels are determined using HTRA1 antibody capture followed by LC-MS.

In some embodiments, the HTRA1 protein levels are determined via LC-MS of a protease, e.g. trypsin, digested protein sample of immune-captured HTRA1 obtained from a sample of vitreous or aqueous humor form the subject. Other proteases which may be used to digest the immuno-captured HTRA1 include LysC, AspN, GluC.

In some embodiments, HTRA1 protein is immunocaptured from the sample using an HTRA1 antibody, such as via an immunocapture Enzyme-Linked Immunosorbant Assay (ELISA), or via western blot.

Comparing the Level of HTRA1 with One or More Reference Samples

As explained above, HTRA1 over-expression is associated with numerous ocular diseases, including AMD, such as early dry AMD, intermediate dry AMD, or advanced dry AMD such as geographic atrophy. The method of the invention in step ii) may therefore comprise a comparison of the HTRA1 levels (e.g. HTRA1 protein levels) obtained in step i) with one or more reference values of the HTRA1 level in a healthy subject (negative control subject) who do not suffer from an ocular disease or have a disease associate polymorphism in the HTRA 1 gene (including the HTRA control region), or an average or mean reference value obtained from a population of such healthy subjects (negative control subject). Alternatively or in addition, step ii) may comprise a comparison of the HTRA1 levels (e.g. HTRA1 protein levels) obtained in step ii) with one or more reference values of the HTRA1 levels in a subject who has been diagnosed with an HTRA1 associated ocular disease, or has been characterized as over-expressing HTRA1, and/or has been characterized as having a disease associate polymorphism in the HTRA 1 gene (including the HTRA control region)—i.e. a positive control. A HTRA1 level which is elevated as compared to the negative control subject, and/or is similar to or equivalent to the positive control value is an indication that the subject is likely to be or is suitable for treatment with the HTRA1 mRNA antagonist.

In a further embodiment, in addition to the positive and/or negative control values, the values obtained previously from the same individual subject may be used. This reference samples may therefore include a historically value, or historical values, obtained previously from the same subject. By comparing the HTRA1 levels obtained in step i) with historical value(s) from the same subject allows the monitoring of the efficacy of the HTRA1 mRNA antagonist therapy, and therefore may be used to determine the likely effective dose of the HTRA1 mRNA antagonist. In this regard, in some embodiments, the aim of HTRA1 mRNA antagonist therapy is to essentially normalize the HTRA1 levels rather than achieve complete inhibition. The method or use of the invention may therefore be used for patient monitoring of HTRA1 levels prior to, during or after the HTRA1 treatment, and for example may be use between administration doses of the HTRA1 mRNA antagonist, for example to allow for modulation of the dosage to optimize the effectiveness of treatment.

### Further Embodiments of the Invention

1. A method for determining the suitability of treatment of a subject for administration with an HTRA1 mRNA antagonist, said method comprising the steps of:

i) determining the level of HTRA1 in a sample of aqueous or vitreous humor obtained from the subject

ii) comparing the level of HTRA1 obtained from step i) with one or more reference samples or reference values;

to determine whether the subject is likely to be, or is suitable for, treatment of with the HTRA1 mRNA antagonist,

wherein the subject is suffering from or is at risk of developing an ocular disorder, such as macular degeneration.

2. The method according to embodiment 1, wherein the level of HTRA1 in the sample of aqueous humor or vitreous humor is determined by quantifying the level of HTRA1 protein in the sample.

3. The method according to embodiment 2, wherein the level of HTRA1 protein is determined using an immuno assay, such as using an HTRA1 specific antibody.

4. The method according to embodiment 3, wherein the level of HTRA1 protein is determined using mass spectrometry, such as via HTRA1 antibody capture followed by LC-MS.

5. The method according to any one of embodiments 1-4, wherein at least one of the reference sample or reference values is obtained from a control subject who is suffering from macular degeneration.

6. The method according to any one of embodiments 1-5, wherein at least one of the reference samples or reference values is obtained from a control subject who is not suffering from macular degeneration.

7. The method according to any one of embodiments 1-6, wherein the at least one of the reference samples is a value previously obtained from the same subject whose suitability of treatment is being assessed.

8. The method according to any one of embodiments 1-7, wherein the reference values is derived from a dataset modeling the correlation between HTRA1 concentration in the retina and the HTRA1 concentration in the aqueous humor or vitreous humor.

9. The method according to any one of embodiments 1-8, wherein the HTRA1 mRNA antagonist is selected from the group consisting of an antisense oligonucleotide targeting HTRA1 mRNA or pre-mRNA, an siRNA targeting HTRA1 mRNA, a ribozyme targeting HTRA1 mRNA or pre-mRNA.

10. The method according to any one of embodiments 1-9 wherein the method is for determining whether the subject has an enhanced HTRA1 mRNA or HTRA1 protein expression in the retina such as retinal epithelial cells.

11. The method according to any one of embodiments 1-10, wherein the subject is suffering from or is at risk of developing an ocular disorder selected from the group consisting of macular degenerative diseases such as age-related macular degeneration (AMD), including wet (exudative) AMD (including early, intermediate, and advanced wet AMD) and dry (nonexudative) AMD (including early, intermediate, and advanced dry AMD (e.g., geographic atrophy (GA)); diabetic retinopathy (DR) and other ischemia-related retinopathies; endophthalmitis; uveitis; choroidal neovascularization (CNV); retinopathy of prematurity (ROP); polypoidal choroidal vasculopathy (PCV); diabetic macular edema; pathological myopia; von Hippel-Lindau disease; histoplasmosis of the eye; Central Retinal Vein Occlusion (CRVO); corneal neovascularization; and retinal neovascularization.

12. The method according to any one of embodiments 1-11, wherein the subject is suffering from or is at risk of developing age-related macular degeneration (AMD), such as AMD selected from the group consisting of wet (exudative) AMD (including early, intermediate, and advanced wet AMD), dry (non-exudative) AMD (including early, intermediate, and advanced dry AMD (e.g., geographic atrophy (GA)); advantageously dry

AMD. 13. The method according to any one of embodiments 1-12, wherein the method is for determining the suitable dose regimen for the administration with the HTRA1 mRNA antagonist.

14. The method according to any one of embodiments 1-13, wherein the HTRA1 mRNA antagonist is an oligonucleotide which comprises a contiguous nucleotide region of 10-30 nucleotides which are fully complementary to a HTRA1 target nucleic acid sequence, such as SEQ ID NO 1 or SEQ ID NO 2.

15. The method according to any one of embodiments 1-14, wherein the HTRA1 mRNA antagonist is or comprises an oligonucleotide which comprises a contiguous nucleotide sequence of at least 12 nucleotides in length which are at least 90& complementary to, such as fully complementary to SEQ ID NO 7 or SEQ ID NO 16.

16. The method according to any one of embodiments 1-15, wherein the HTRA1 mRNA antagonist is or comprises an antisense oligonucleotide, such as an LNA gapmer oligonucleotide.

17. The method according to any one of embodiments 1-16, wherein the HTRA1 mRNA antagonist is selected from the group consisting of

(SEQ ID NO 13, #13,1)
5′ TₛAₛTₛtₛtₛaₛcₛcₛtₛgₛgₛtₛTₛGₛTₛT 3′
(SEQ ID NO 15, #15,3)
5′ AₛtₛAₛTₛtₛtₛaₛcₛcₛtₛgₛgₛtₛTₛGₛTₛT 3′
(SEQ ID NO 17, #17)
5′ CₛTₛTₛCₛtₛtₛcₛtₛaₛtₛcₛtₛaₛ^{m}cₛgₛcₛAₛT 3′,
(SEQ ID NO 18, #18)
5′ TₛAₛCₛTₛtₛtₛaₛaₛtₛaₛgₛcₛTₛCₛAₛA 3′;
and
(SEQ ID NO 19, #19)
5′ TₛTₛCₛtₛaₛtₛcₛtₛaₛ^{m}cₛgₛcₛaₛTₛTₛG 3′,

wherein a capital letter represents an beta-D oxy LNA nucleoside unit, a lower case letter represents a DNA nucleoside unit, subscript s represents a phosphorothioate internucleoside linkage, ^{m}c represents 5 methyl cytosine DNA nucleosides, and all LNA cytosines are 5-methyl cytosine; or a pharmaceutically acceptable salt thereof.

18. A method for treating a subject suffering from or at risk of developing macular degeneration, said method comprising performing the method according to any one of embodiments 1-17, and administering to the subject an effective amount of an HTRA1 mRNA antagonist.

19. The method according to any one of embodiments 1-17, wherein the HTRA1 mRNA antagonist is for administration via intra-vitral administration.

20. Use of an HTRA1 antibody as a companion diagnostic for a HTRA1 RNA antagonist therapeutic.

21. Use of an HTRA1 antibody as a biomarker for the efficacy for an HTRA1 RNA antagonist therapeutic, such as an HTRA1 mRNA antagonist, such as according to any one of the preceding embodiments.

22. Use of an HTRA1 antibody in the detection of HTRA1 levels in an aqueous humor or vitreous humor sample obtained from a subject who is undergoing treatment with an HTRA1 mRNA antagonist, or is being assessed for suitability of treatment with an HTRA1 mRNA antagonist, such as according to any one of the preceding embodiments.

22. Use of a biomarker for determining the likely response of a subject to a therapeutic agent comprising a HTRA1 mRNA antagonist, such as according to any one of the preceding embodiments, wherein the biomarker comprises an elevated level of HTRA1 in a biological sample obtained from the aqueous humor or vitreous humor of the subject, as compared to the level of HTRA1 obtained from a reference sample from a healthy subject.

### EXAMPLES

Oligonucleotide Synthesis

Oligonucleotide synthesis is generally known in the art. Below is a protocol which may be applied. The oligonucleotides of the present invention may have been produced by slightly varying methods in terms of apparatus, support and concentrations used.

Oligonucleotides are synthesized on uridine universal supports using the phosphoramidite approach on an Oligomaker 48 at 1 μmol scale. At the end of the synthesis, the oligonucleotides are cleaved from the solid support using aqueous ammonia for 5-16 hours at 60° C. The oligonucleotides are purified by reverse phase HPLC (RP-HPLC) or by solid phase extractions and characterized by UPLC, and the molecular mass is further confirmed by ESI-MS.

Elongation of the Oligonucleotide:

The coupling of β-cyanoethyl-phosphoramidites (DNA-A(Bz), DNA-G(ibu), DNA-C(Bz), DNA-T, LNA-5-methyl-C(Bz), LNA-A(Bz), LNA-G(dmf), LNA-T) is performed by using a solution of 0.1 M of the 5′-O-DMT-protected amidite in acetonitrile and DCI (4,5-dicyanoimidazole) in acetonitrile (0.25 M) as activator. For the final cycle a phosphoramidite with desired modifications can be used, e.g. a C6 linker for attaching a conjugate group or a conjugate group as such. Thiolation for introduction of phosphorthioate linkages is carried out by using xanthane hydride (0.01 M in acetonitrile/pyridine 9:1). Phosphordiester linkages can be introduced using 0.02 M iodine in THF/Pyridine/water 7:2:1. The rest of the reagents are the ones typically used for oligonucleotide synthesis.

For post solid phase synthesis conjugation a commercially available C6 aminolinker phorphoramidite can be used in the last cycle of the solid phase synthesis and after deprotection and cleavage from the solid support the aminolinked deprotected oligonucleotide is isolated. The conjugates are introduced via activation of the functional group using standard synthesis methods.

Purification by RP-HPLC:

The crude compounds are purified by preparative RP-HPLC on a Phenomenex Jupiter C18 10p 150×10 mm column. 0.1 M ammonium acetate pH 8 and acetonitrile is used as buffers at a flow rate of 5 mL/min. The collected fractions are lyophilized to give the purified compound typically as a white solid.

### Example 1. Cynomolgus Monkey (Non-Human Primate, NHP) In Vivo Pharmacokinetics and Pharmacodynamics (PK/PD) Study, 21 Days of Treatment, Intravitreal (IVT) Injection, Single Dose

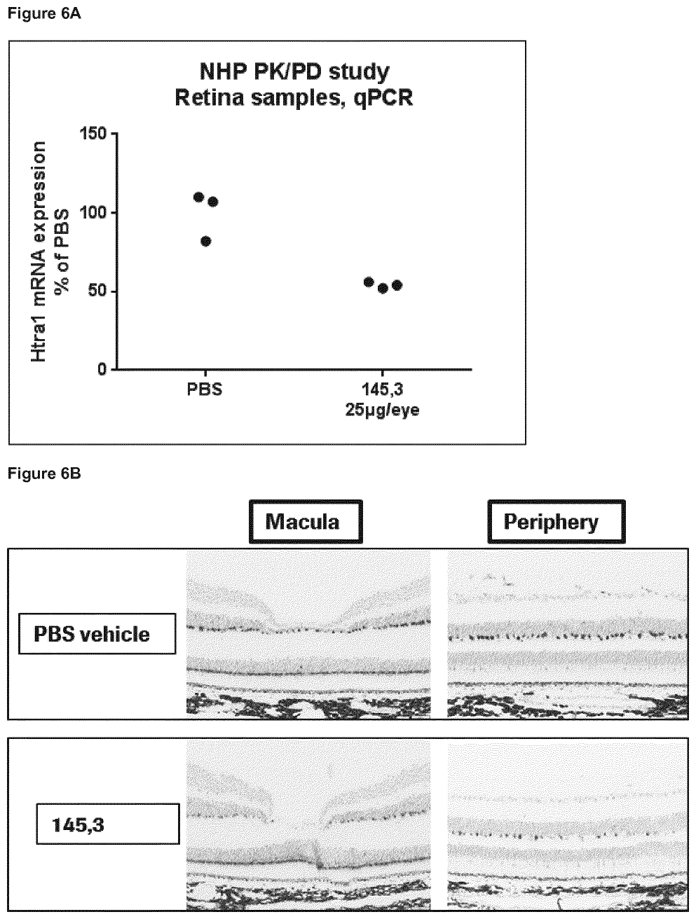

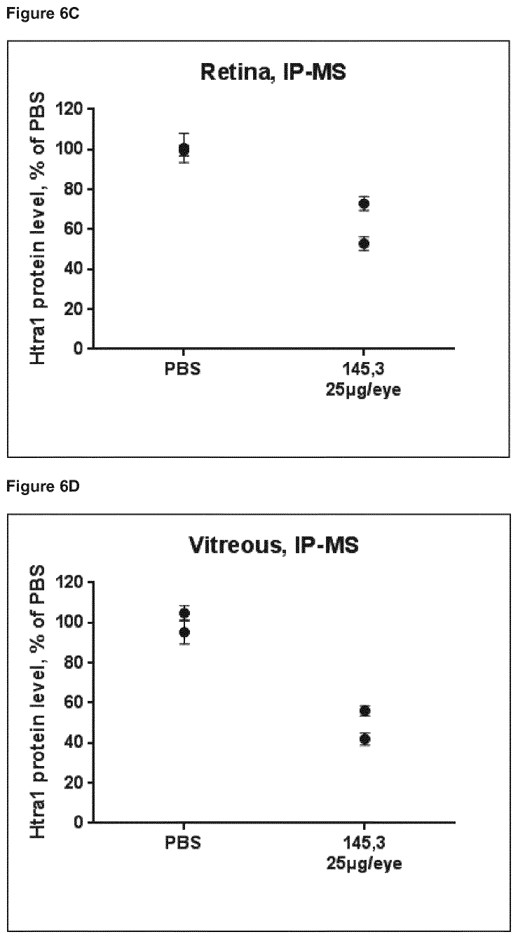

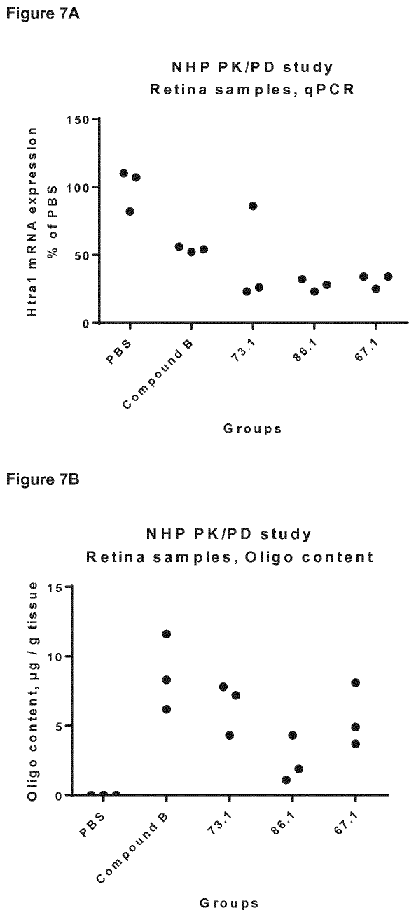

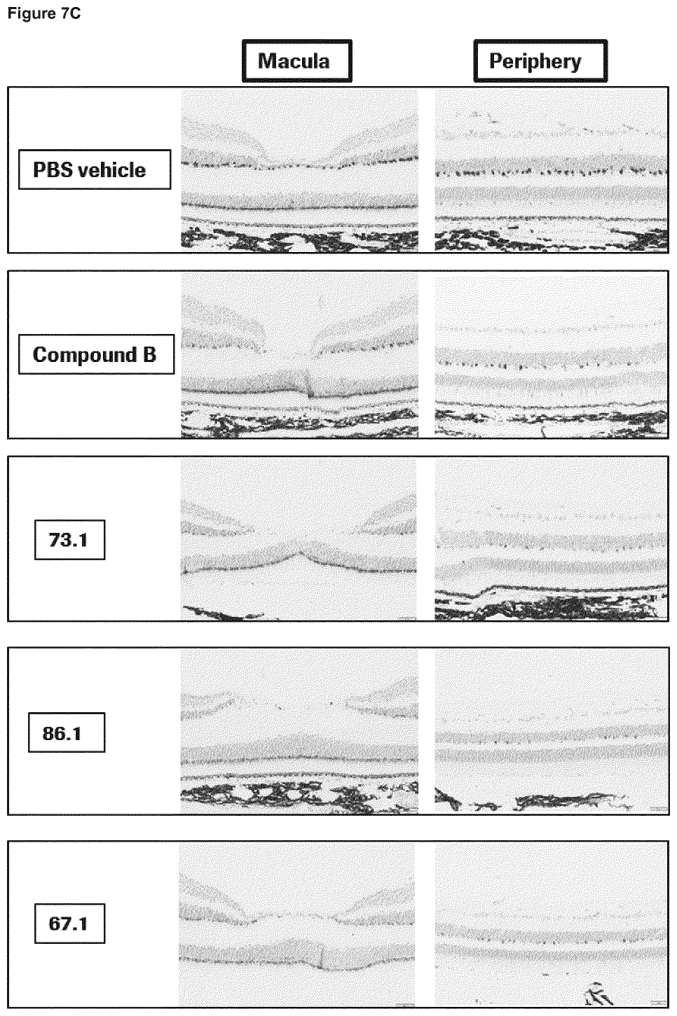

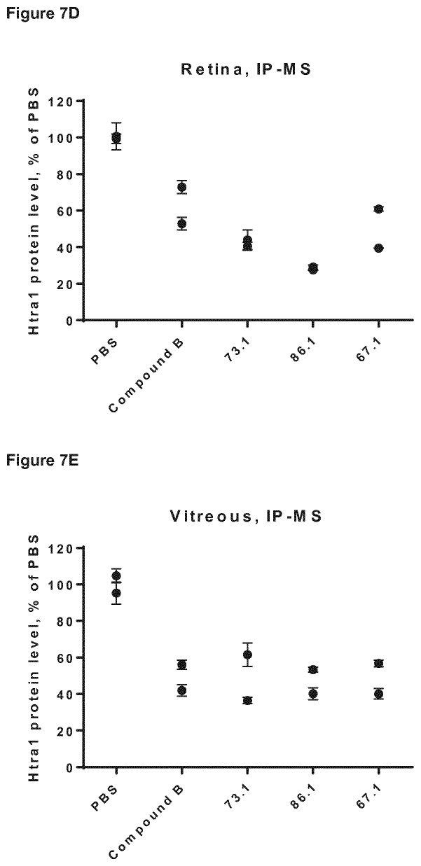

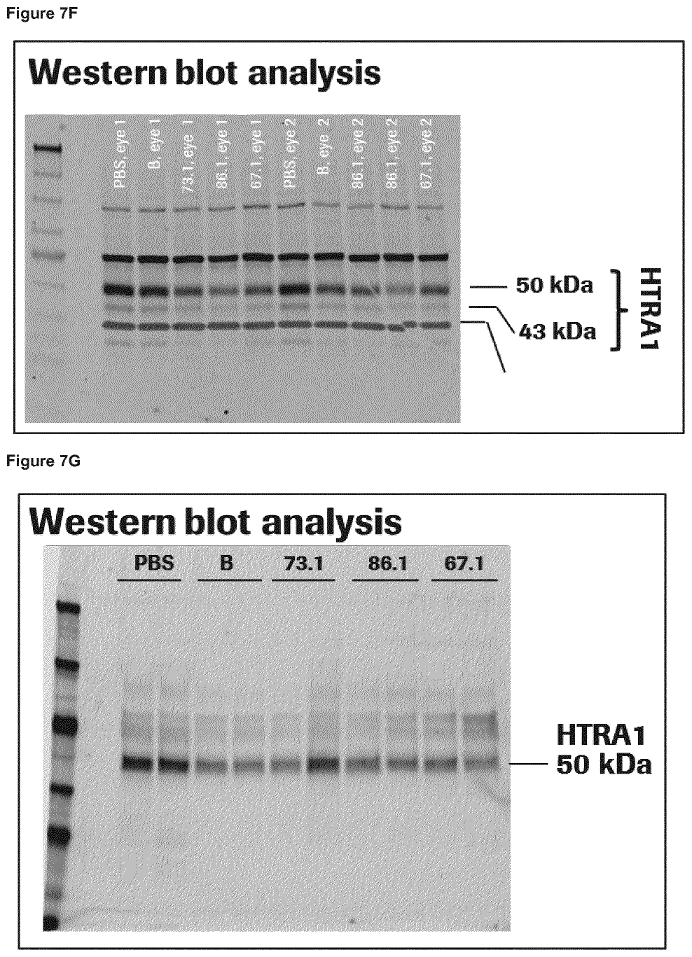

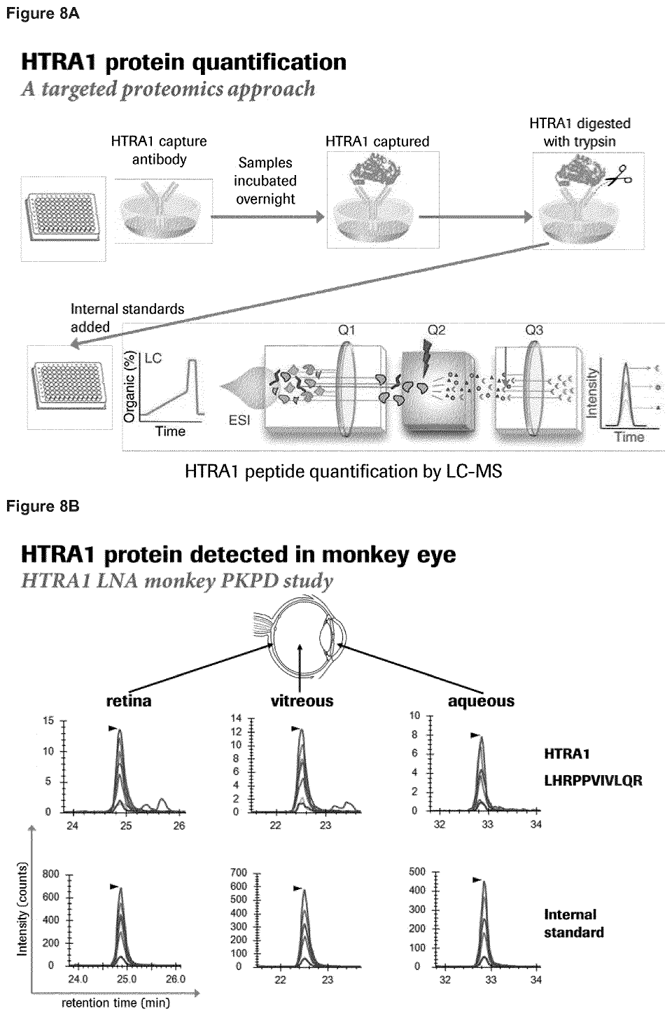

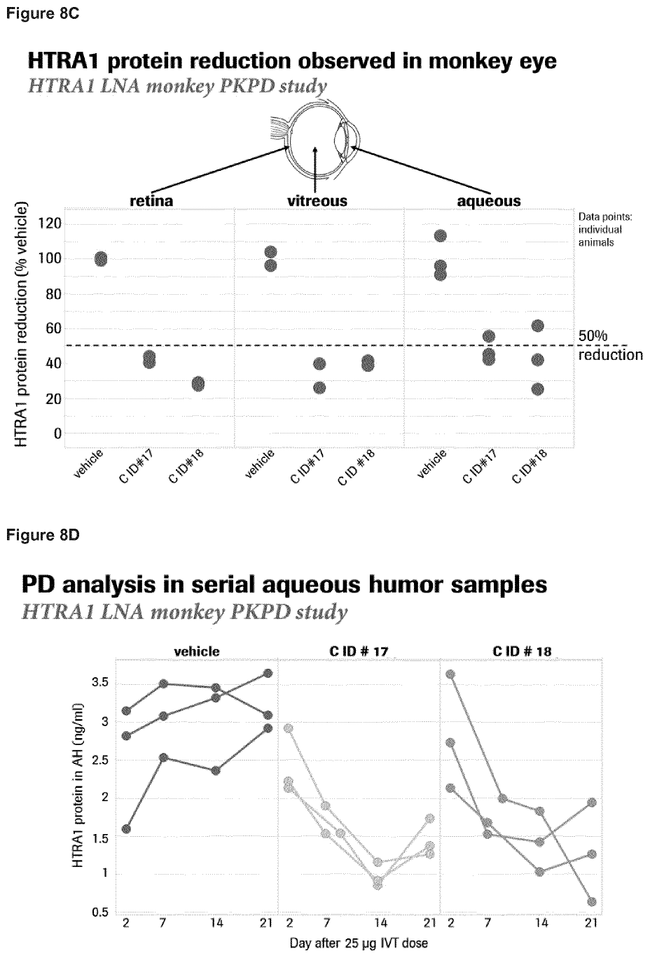

Knockdown was observed for 1 selected HTRA1 LNA oligonucleotide, 15.3, targeting the “hotspot” in human HTRA1 pre-mRNA between position 33042-33064 both at mRNA in the retina and at protein level in the retina and in the vitreous (see FIG. 6).

Animals

All experiments were performed on Cynomolgus monkeys (Macaca fascicularis).

Compounds and Dosing Procedures

Buprenorphine analgesia was administered prior to, and two days after test compound injection. The animals were anesthetized with an intramuscular injection of ketamine and xylazine. The test item and negative control (PBS) were administered intravitreally in both eyes of anesthetized animals (50 μL per administration) on study day 1 after local application of tetracaine anesthetic.

Euthanasia

At the end of the in-life phase (Day 22) all monkeys were euthanized by intraperitoneal an overdose injection of pentobarbital.

Oligo content measurement and quantification of Htra1 RNA expression by qPCR Immediately after euthanasia, eye tissues were quickly and carefully dissected out on ice and stored at −80° C. until shipment. Retina sample was lysed in 700 μL MagNa Pure 96 LC RNA Isolation Tissue buffer and homogenized by adding 1 stainless steel bead per 2 ml tube 2×1.5 min using a precellys evolution homogenizer followed by 30 min incubation at RT. The samples were centrifuged, 13000 rpm, 5 min. Half was set aside for bioanalysis and for the other half, RNA extraction was continued directly.

For bioanalysis, the samples were diluted 10-50 fold for oligo content measurements with a hybridization ELISA method. A biotinylated LNA-capture probe and a digoxigenin-conjugated LNA-detection probe (both 35 nM in 5×SSCT, each complementary to one end of the LNA oligonucleotide to be detected) was mixed with the diluted homogenates or relevant standards, incubated for 30 minutes at RT and then added to a streptavidine-coated ELISA plates (Nunc cat. no. 436014).

The plates were incubated for 1 hour at RT, washed in 2×SSCT (300 mM sodium chloride, 30 mM sodium citrate and 0.05% v/v Tween-20, pH 7.0) The captured LNA duplexes were detected using an anti-DIG antibodies conjugated with alkaline phosphatase (Roche Applied Science cat. No. 11093274910) and an alkaline phosphatase substrate system (Blue Phos substrate, KPL product code 50-88-00). The amount of oligo complexes was measured as absorbance at 615 nm on a Biotek reader.

For RNA extraction, cellular RNA large volume kit (05467535001, Roche) was used in the MagNA Pure 96 system with the program: Tissue FF standard LV3.1 according to the instructions of the manufacturer, including DNAse treatment. RNA quality control and concentration were measured with an Eon reader (Biotek). The RNA concentration was normalized across samples, and subsequent cDNA synthesis and qPCR was performed in a one-step reaction using qScript XLT one-step RT-qPCR ToughMix Low ROX, 95134-100 (Quanta Biosciences). The following TaqMan primer assays were used in singplex reactions: Htra1, Mf01016150_, Mf01016152_m1 and Rh02799527_m1 and housekeeping genes, ARFGAP2, Mf01058488_g1 and Rh01058485_m1, and ARL1, Mf02795431_m1, from Life Technologies. The qPCR analyses were run on a ViiA7 machine (Life Technologies). Eyes/group: n=3 eyes. Each eye was treated as an individual sample. The relative Htra1 mRNA expression level is shown as % of control (PBS).

Histology

Eyeballs were removed and fixed in 10% neutral buffered formalin for 24 hours, trimmed and embedded in paraffin.

For ISH analysis, sections of formalin-fixed, paraffin-embedded retina tissue 4 μm thick were processed using the fully automated Ventana Discovery ULTRA Staining Module (Procedure: mRNA Discovery Ultra Red 4.0—v0.00.0152) using the RNAscope 2.5 VS Probe-Mmu-HTRA1, REF 486979, Advanced Cell Diagnostics, Inc. Chromogen used is Fastred, Hematoxylin II counterstain.

HTRA1 Protein Quantification Using a Plate-Based Immunoprecipitation Mass Spectrometry (IP-MS) Approach

Sample Preparation, Retina

Retinas were homogenized in 4 volumes (w/v) of RIPA buffer (50 mM Tris-HCl, pH 7.4, 150 mM NaCl, 0.25% deoxycholic acid, 1% NP-40, 1 mM EDTA, Millipore) with protease inhibitors (Complete EDTA-free, Roche) using a Precellys 24 (5500, 15 s, 2 cycles). Homogenates were centrifuged (13,000 rpm, 3 min) and the protein contents of the supernatants determined (Pierce BCA protein assay)

Sample Preparation, Vitreous

Vitreous humors (300 μl) were diluted with 5×RIPA buffer (final concentration: 50 mM Tris-HCl, pH 7.4, 150 mM NaCl, 0.25% deoxycholic acid, 1% NP-40, 1 mM EDTA) with protease inhibitors (Complete EDTA-free, Roche) and homogenized using a Precellys 24 (5500, 15 s, 2 cycles). Homogenates were centrifuged (13,000 rpm, 3 min) and the protein contents of the supernatants determined (Pierce BCA protein assay)

Plate-Based HTRA1 Immunoprecipitation and Tryptic Digest

A 96 well plate (Nunc MaxiSorp) was coated with anti-HTRA1 mouse monoclonal antibody (R&D MAB2916, 500 ng/well in 50 μl PBS) and incubated overnight at 4° C. The plate was washed twice with PBS (200 μl) and blocked with 3% (w/v) BSA in PBS for 30 min at 20° C. followed by two PBS washes. Samples (75 μg retina, 100 μg vitreous in 50 μl PBS) were randomized and added to the plate followed by overnight incubation at 4° C. on a shaker (150 rpm). The plate was then washed twice with PBS and once with water. 10 mM DTT in 50 mM TEAB (30 μl) were then added to each well followed by incubation for 1 h at 20° C. to reduce cysteine sulfhydryls. 150 mM iodoacetamide in 50 mM TEAB (5 μl) were then added to each well followed by incubation for 30 min at 20° C. in the dark in order to block cysteine sulfhydryls. 10 μl Digestion solution were added to each well (final concentrations: 1.24 ng/μl trypsin, 20 fmol/μl BSA peptides, 26 fmol/μl isotope-labeled HTRA1 peptides, 1 fmol/μl iRT peptides, Biognosys) followed by incubation overnight at 20° C.

HTRA1 Peptide Quantification by Targeted Mass Spectrometry (Selected Reaction Monitoring, SRM)

Mass spectrometry analysis was performed on an Ultimate RSLCnano LC coupled to a TSQ Quantiva triple quadrupole mass spectrometer (Thermo Scientific). Samples (20 μL) were injected directly from the 96 well plate used for IP and loaded at 5 μL/min for 6 min onto a Acclaim Pepmap 100 trap column (100 μm×2 cm, C18, 5 μm, 100 Å, Thermo Scientific) in loading buffer (0.5% v/v formic acid, 2% v/v ACN). Peptides were then resolved on a PepMap Easy-SPRAY analytical column (75 μm×15 cm, 3 μm, 100 Å, Thermo Scientific) with integrated electrospray emitter heated to 40° C. using the following gradient at a flow rate of 250 nL/min: 6 min, 98% buffer A (2% ACN, 0.1% formic acid), 2% buffer B (ACN+0.1% formic acid); 36 min, 30% buffer B; 41 min, 60% buffer B; 43 min, 80% buffer B; 49 min, 80% buffer B; 50 min, 2% buffer B. The TSQ Quantiva was operated in SRM mode with the following parameters: cycle time, 1.5 s; spray voltage, 1800 V; collision gas pressure, 2 mTorr; Q1 and Q3 resolution, 0.7 FWHM; ion transfer tube temperature 300° C. SRM transitions were acquired for the HTRA1 peptide “LHRPPVIVLQR” and an isotope labelled (L-[U-130, U-15N]R) synthetic version, which was used an internal standard. Data analysis was performed using Skyline version 3.6.

### Example 2: Cynomolgus Monkey In Vivo Pharmacokinetics and Pharmacodynamics Study, 21 Days of Treatment, Intravitreal (IVT) Injection, Single Dose

Knock down was observed for 3 HTRA1 LNA oligonucleotides targeting the “hotspot” in human HTRA1 pre-mRNA between position 53113-53384 both at mRNA in the retina and at protein level in the retina and in the vitreous.

Animals

All experiments were performed on Cynomolgus monkeys (Macaca fascicularis).

Four animals were included in each group of the study, 20 in total.

Compounds and Dosing Procedures

Buprenorphine analgesia was administered prior to, and two days after test compound injection. The animals were anesthetized with an intramuscular injection of ketamine and xylazine. The test item and negative control (PBS) were administered intravitreally in both eyes of anesthetized animals (50 μL per administration) on study day 1 after local application of tetracaine anesthetic.

Euthanasia

At the end of the in-life phase (Day 22) all monkeys were euthanized by intraperitoneal an overdose injection of pentobarbital.

Oligo content measurement and quantification of Htra1 RNA expression by qPCR Immediately after euthanasia, eye tissues were quickly and carefully dissected out on ice and stored at −80° C. until shipment. Retina sample was lysed in 700 μL MagNa Pure 96 LC RNA Isolation Tissue buffer and homogenized by adding 1 stainless steel bead per 2 ml tube 2×1.5 min using a precellys evolution homogenizer followed by 30 min incubation at RT. The samples were centrifuged, 13000 rpm, 5 min. Half was set aside for bioanalysis and for the other half, RNA extraction was continued directly.

For bioanalysis, the samples were diluted 10-50 fold for oligo content measurements with a hybridization ELISA method. A biotinylated LNA-capture probe and a digoxigenin-conjugated LNA-detection probe (both 35 nM in 5×SSCT, each complementary to one end of the LNA oligonucleotide to be detected) was mixed with the diluted homogenates or relevant standards, incubated for 30 minutes at RT and then added to a streptavidine-coated ELISA plates (Nunc cat. no. 436014).

The plates were incubated for 1 hour at RT, washed in 2×SSCT (300 mM sodium chloride, 30 mM sodium citrate and 0.05% v/v Tween-20, pH 7.0) The captured LNA duplexes were detected using an anti-DIG antibodies conjugated with alkaline phosphatase (Roche Applied Science cat. No. 11093274910) and an alkaline phosphatase substrate system (Blue Phos substrate, KPL product code 50-88-00). The amount of oligo complexes was measured as absorbance at 615 nm on a Biotek reader.

For RNA extraction, cellular RNA large volume kit (05467535001, Roche) was used in the MagNA Pure 96 system with the program: Tissue FF standard LV3.1 according to the instructions of the manufacturer, including DNAse treatment. RNA quality control and concentration were measured with an Eon reader (Biotek). The RNA concentration was normalized across samples, and subsequent cDNA synthesis and qPCR was performed in a one-step reaction using qScript XLT one-step RT-qPCR ToughMix Low ROX, 95134-100 (Quanta Biosciences). The following TaqMan primer assays were used in singplex reactions: Htra1, Mf01016150_, Mf01016152_m1 and Rh02799527_m1 and housekeeping genes, ARFGAP2, Mf01058488_g1 and Rh01058485_m1, and ARL1, Mf02795431_m1, from Life Technologies. The qPCR analyses were run on a ViiA7 machine (Life Technologies). Eyes/group: n=3 eyes. Each eye was treated as an individual sample. The relative Htra1 mRNA expression level is shown as % of control (PBS).

Histology

Eyeballs were removed and fixed in 10% neutral buffered formalin for 24 hours, trimmed and embedded in paraffin.

For ISH analysis, sections of formalin-fixed, paraffin-embedded cyno retina tissue 4 μm thick were processed using the fully automated Ventana Discovery ULTRA Staining Module (Procedure: mRNA Discovery Ultra Red 4.0—v0.00.0152) using the RNAscope 2.5 VS Probe-Mmu-HTRA1, REF 486979, Advanced Cell Diagnostics, Inc. Chromogen used is Fastred, Hematoxylin II counterstain.

HTRA1 Protein Quantification Using a Plate-Based Immunoprecipitation Mass Spectrometry (IP-MS) Approach

Sample Preparation, Retina

Retinas were homogenized in 4 volumes (w/v) of RIPA buffer (50 mM Tris-HCl, pH 7.4, 150 mM NaCl, 0.25% deoxycholic acid, 1% NP-40, 1 mM EDTA, Millipore) with protease inhibitors (Complete EDTA-free, Roche) using a Precellys 24 (5500, 15 s, 2 cycles). Homogenates were centrifuged (13,000 rpm, 3 min) and the protein contents of the supernatants determined (Pierce BCA protein assay)

Sample Preparation, Vitreous

Vitreous humors (300 μl) were diluted with 5×RIPA buffer (final concentration: 50 mM Tris-HCl, pH 7.4, 150 mM NaCl, 0.25% deoxycholic acid, 1% NP-40, 1 mM EDTA) with protease inhibitors (Complete EDTA-free, Roche) and homogenized using a Precellys 24 (5500, 15 s, 2 cycles). Homogenates were centrifuged (13,000 rpm, 3 min) and the protein contents of the supernatants determined (Pierce BCA protein assay)

Plate-Based HTRA1 Immunoprecipitation and Tryptic Digest

A 96 well plate (Nunc MaxiSorp) was coated with anti-HTRA1 mouse monoclonal antibody (R&D MAB2916, 500 ng/well in 50 μl PBS) and incubated overnight at 4° C. The plate was washed twice with PBS (200 μl) and blocked with 3% (w/v) BSA in PBS for 30 min at 20° C. followed by two PBS washes. Samples (75 μg retina, 100 μg vitreous in 50 μl PBS) were randomized and added to the plate followed by overnight incubation at 4° C. on a shaker (150 rpm). The plate was then washed twice with PBS and once with water. 10 mM DTT in 50 mM TEAB (30 μl) were then added to each well followed by incubation for 1 h at 20° C. to reduce cysteine sulfhydryls. 150 mM iodoacetamide in 50 mM TEAB (5 μl) were then added to each well followed by incubation for 30 min at 20° C. in the dark in order to block cysteine sulfhydryls. 10 μl Digestion solution were added to each well (final concentrations: 1.24 ng/μl trypsin, 20 fmol/μl BSA peptides, 26 fmol/μl isotope-labeled HTRA1 peptides, 1 fmol/μl iRT peptides, Biognosys) followed by incubation overnight at 20° C.

HTRA1 Peptide Quantification by Targeted Mass Spectrometry (Selected Reaction Monitoring, SRM)

Mass spectrometry analysis was performed on an Ultimate RSLCnano LC coupled to a TSQ Quantiva triple quadrupole mass spectrometer (Thermo Scientific). Samples (20 μL) were injected directly from the 96 well plate used for IP and loaded at 5 μL/min for 6 min onto a Acclaim Pepmap 100 trap column (100 μm×2 cm, C18, 5 μm, 100 Å, Thermo Scientific) in loading buffer (0.5% v/v formic acid, 2% v/v ACN). Peptides were then resolved on a PepMap Easy-SPRAY analytical column (75 μm×15 cm, 3 μm, 100 Å, Thermo Scientific) with integrated electrospray emitter heated to 40° C. using the following gradient at a flow rate of 250 nL/min: 6 min, 98% buffer A (2% ACN, 0.1% formic acid), 2% buffer B (ACN+0.1% formic acid); 36 min, 30% buffer B; 41 min, 60% buffer B; 43 min, 80% buffer B; 49 min, 80% buffer B; 50 min, 2% buffer B. The TSQ Quantiva was operated in SRM mode with the following parameters: cycle time, 1.5 s; spray voltage, 1800 V; collision gas pressure, 2 mTorr; Q1 and Q3 resolution, 0.7 FWHM; ion transfer tube temperature 300° C. SRM transitions were acquired for the HTRA1 peptide “LHRPPVIVLQR” and an isotope labelled (L-[U-13C, U-15N]R) synthetic version, which was used an internal standard.

Data analysis was performed using Skyline version 3.6.

Western Blot

Dissected retina sample in 0.5 Precellyses tubes (CK14-0.5 ml, Bertin Technologies) were lysed and homogenized in RIPA lysis buffer (20-188, Milipore) with protease inhibitors (Complete EDTA-free Proteases-Inhibitor Mini, 11 836 170 001, Roche).

Vitreous sample were added to a 0.5 Precellyses tubes (CK14-0.5 ml, Bertin Technologies) were lysed and homogenized in ¼×RIPA lysis buffer (20-188, Milipore) with protease inhibitors (Complete EDTA-free Proteases-Inhibitor Mini, 11 836 170 001, Roche).

Samples (retina 20 μg protein, vitreous 40 μg protein) were analyzed on 4-15% gradient gel (#567-8084 Bio-Rad) under reducing conditions and transferred on Nitrocellulose (#170-4159 Bio-Rad) using a Trans-Blot Turbo Device from Bio-Rad.

Primary antibodies: Rabbit anti human HTRA1 (SF1) was a kind gift of Sascha Fauser (University of Cologne), mouse anti human Gapdh (#98795 Sigma-Aldrich). Secondary antibody: goat anti rabbit 800 CW and goat anti mouse 680RD were from Li-Cor Blot was imaged and analyzed on an Odyssee CLX from Li-Cor.

### Example 3—Cynomolgus Monkey In Vivo Assessment: HTRA1 Protein Determination in Aqueous Humor and Comparison to HTRA1 mRNA and Protein Inhibition in Retina

Experimental Methodology: See Example 2. Aqueous humor samples were taken and samples were prepared as according to example 2 vitreous humor samples. Cynomolgus Monkey Aqueous humor samples (AH) were analyzed with a size-based assay on a Analytical Methodology: Capillary Electrophoresis System (Peggy Sue™, Proteinsimple) Samples were thawed on ice and used undiluted. For quantification, recombinant HTRA1-S328A mutant (Origene #TP700208). Preparation was as described by the provider. Primary rabbit anti-human HTRA Antibody SF1 was provided by Prof. Dr. Sascha Fauser and used diluted 1:300. All other reagents were from Proteinsimple.

Samples were processed in technical triplicate, calibration curve in duplicate using a 12-230 kDa Separation module. Area under the peak was computed and analyzed using Xlfit (IDBS software).

Results

| Group | mRNA_retina | protein_retina | protein_AH |
|---|---|---|---|
| PBS | 82 | 101 | 95 |
| PBS | 107 | 99 | 118 |
| #15, 3 | 56 | 73 | 51 |
| #15, 3 | 52 | 53 | 68 |
| #17 | 23 | 41 | 47 |
| #17 | 26 | 44 | 44 |
| #18 | 32 | 29 | 44 |
| #18 | 23 | 28 | 64 |
| #19 | 34 | 39 | 44 |
| #19 | 34 | 61 | 42 |

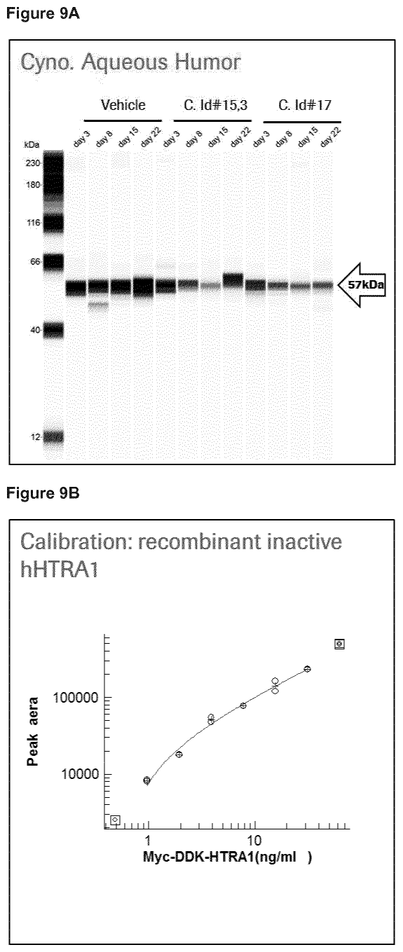

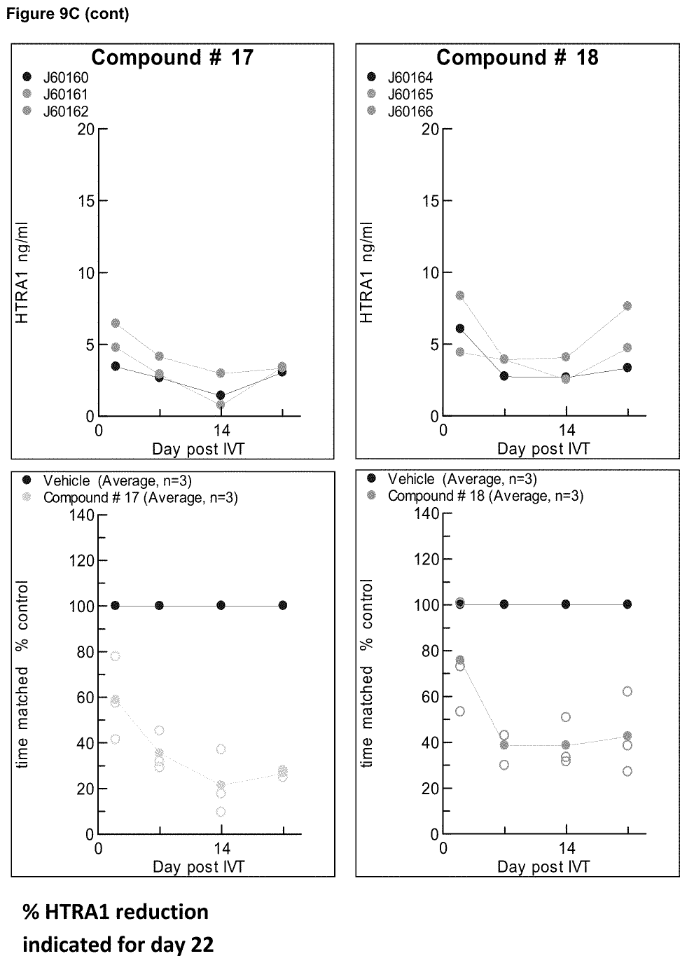

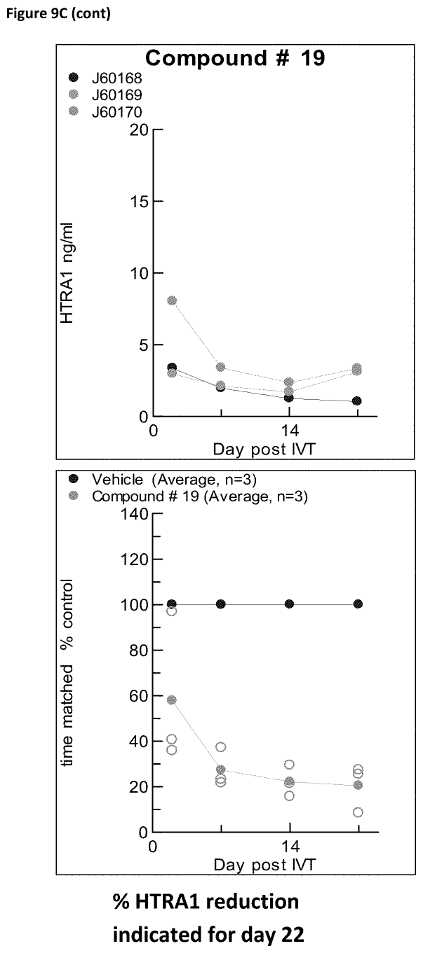

FIG. 9A shows a visualization of the HTRA1 protein levels in the aqueous humor of monkeys administered with compounds #15.3 and #17, with samples taken at days 3, 8, 15, and 22 post-injection. FIG. 9B provides the calibration curve used in calculating HTRA1 protein levels. FIG. 9C provides the calculated HTRA1 levels from aqueous humor from individual animal was plotted against time post injection.

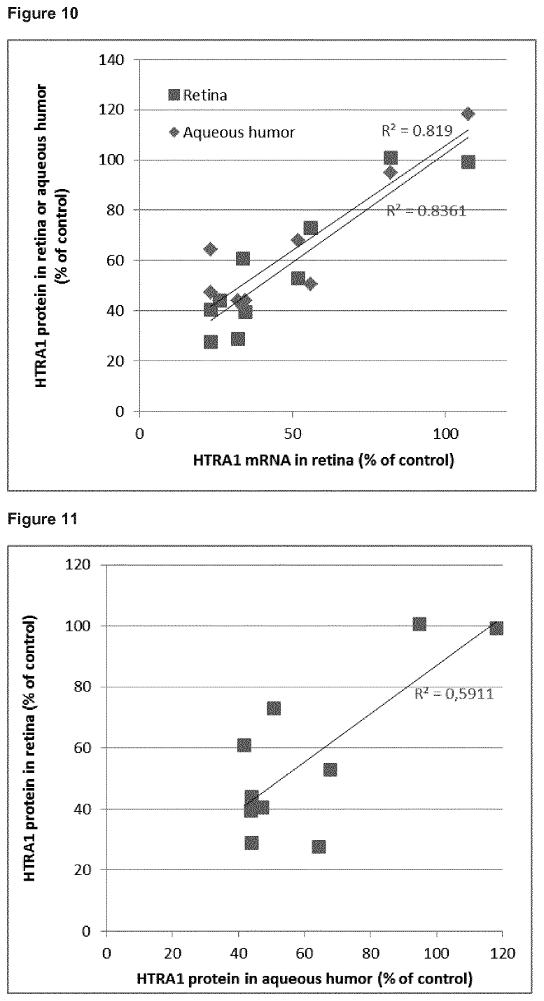

FIG. 10 illustrates a direct correlation between the level of HTRA1 protein in the aqueous humor and the level of HTRA1 mRNA in the retina. Aqueous humor HTRA1 protein levels may therefore be used as a biomarker for HTRA1 retina mRNA levels or HTRA1 retinal mRNA inhibition.

FIG. 11 illustrates that there is also a correlation between HTRA1 protein levels in retina and the HTRA1 protein levels in aqueous humor, although the correlation was not, in this experiment, as strong as the correlation between HTRA1 mRNA inhibition in the retina and HTRA1 protein levels in the aqueous humor, indicating that aqueous humor HTRA1 protein levels are particularly suited as biomarker for HTRA1 mRNA antagonists.

### Example 4: Cynomolgus Monkey In Vivo Pharmacokinetics and Pharmacodynamics Study, 36 Days of Treatment, Intravitreal (IVT) Injection, Single Dose

Dynamic of HTRA1 protein in the aqueous humor of HTRA1 LNA exposed animals was investigated over 36 days and residual HTRA1 protein content examined post mortem in different eye tissue compartments (vitreous, neural retina, retinal pigment epithelium (RPE) and Choroid). HTRA1 mRNA suppression was assessed in terminal samples.

Animals

All experiments were performed on male Cynomolgus monkeys (Macaca fascicularis), in the facilities of Charles Rivers Laboratories Montreal ULC at Sennville, Canada.

Compounds and Dosing Procedures

A topical antibiotic (tobramycin) was applied to both eyes twice on the day before and twice on the day after each injection. Prior to dosing, fasted animals received an intramuscular injection of a sedative cocktail of ketamine (5 mg/kg) and dexmetedomidine (0.01 mg/kg) followed by isoflurane/oxygen mix through a mask. A topical anesthetic (0.5% proparacaine) was instilled in each eye before bilateral intravitreal injection of 100 μg test item in 50μl or vehicle (phosphate buffered saline). Mydriatic drops (1% tropicamide) were applied to each eye as needed. Following completion of the dosing procedure, animals received an intramuscular injection of 0.1 mg/kg atipamezole, a reversal agent for dexmetedomidine On day 0 of the procedure 4 animals were bilaterally injected with 100 μg HTRA1 LNA #18 in 50μl and 4 animals with the same volume of vehicle.

Aqueous Humor Sampling

Animals were assigned into two groups comprising each two controls and two treated monkeys. Aqueous humor samples were collected at baseline (all animals); day 3 and day 18, (group1); day 11, day 25, (group2) day 32 (all animals) and after euthanasia day 36. Animals were anesthetized according to the same procedure as for compound application and 30-40 μl aqueous humor sampled after mydriasis, and stored frozen −80° C.

Euthanasia

On day 36 Fasted animals were anesthetized with ketamine and isoflurane, aorta and vena cava clamped and a transcardial perfusion of the upper body performed with phosphate buffered saline. The flushed eyes balls were removed and cleaned from adherent tissue and 200μl aqueous humor sampled. Left eye was further processed for RNA preparation and right eye for protein analysis. HTRA1 protein in aqueous humor was measured from both left and right eye at necropsy.

Sample Processing for HTRA1 Protein Analysis

For terminal collection, up to 0.2 ml aqueous humor was harvested with an ultrafine insulin syringe with a 30G, ½″ needle. The eye ball was cleaned from adherent muscle and opened frontally by a circumferential incision 3 mm posterior to the corneal limbus. Vitreous was removed, homogenized by forcing the tissue (3 passes) through a 3 cc syringe with no needle, centrifuged (3 min 16,000×g at 4° C.) and stored frozen. Neural retina was carefully peeled off from the underling RPE and snap frozen on dry ice. The opened cup was then maintained frontal side up, filed with 1m1 RIPA buffer (Millipore, 50 mM Tris-HCl, pH 7.4, 150 mM NaCl, 0.25% deoxycholic acid, 1% NP-40, 1 mM EDTA) containing protease inhibitors (cOmplete™ EDTA free protease inhibitor cocktail, mini, Roche, 1 tablet/10 ml), shook on a rotary platform (200 rpm) for 5 minutes. The resulting RPE lysate was cleared by centrifugation (3 min 16,000×g at 4° C.) and stored frozen. The remaining posterior eye cup was flushed away with phosphate buffered saline, four incision made allowing to lay the tissue flat and Bruch's membrane and the adhering choroid was peeled off. Choroid was homogenized in 4 ml per gram tissue RIPA buffer containing protease inhibitor using a Geno Grinder (1500 rpm for 2 min) 3 times with intervening cooling on ice. Debris were removed by centrifugation and extract stored (3 min 16,000×g at 4° C.).

Vitreous humors were diluted with 5×RIPA buffer (final concentration: 50 mM TrisHCl, pH 7.4, 150 mM NaCl, 0.25% deoxycholic acid, 1% NP-40, 1 mM EDTA) with protease inhibitors (Complete EDTA-free, Roche) and homogenized using a Precellys 24 (5500 rpm, 15 s, 2 cycles). Homogenates were centrifuged (16,000×g, 3 min). Protein content of neural retina and choroid extract was measured using bicinchoninic acid method and reagents from Pierce (Rockford USA) using serum albumin as standard.

Sample Processing for RNA Preparation

For RNA preparation initial dissection steps were similar to the protein procedure. The peeled retina was transferred to a homogenization vessel, 10μl/mg tissue homogeneization buffer was added (Maxwell RNA isolation kit, Promega). The material was processed in a Geno grinder for three cycles of 2 minutes at 1500 rpm. The resulting lysate stored −80° C. until further processing.

After neural retina removal the opened cup was filled with RNA protect Cell reagent (Qiagen), shaken on a rotary platform (400 rpm) at 4° C. The resulting RPE cell suspension was harvested after 10 minutes, centrifuged 700 g for 5 minutes and lysed in 200 μl Maxwell homogenization buffer. The lysate was stored −80° C. until further processing.

Fresh RNA protect was added to the eye cup which was shaken for further 10 minutes to remove remaining RPE cells and discarded. Four incisions were made allowing to lay the tissue flat and Bruch's membrane and the adhering choroid was peeled off and stored frozen until further processing. For homogenization, frozen choroid was added without thawing to Promega homogenization buffer (10μl/mg tissue) and processed in a Tissue Lyser II (Retsch) for 3 cycles of 2 min at 20 Hz.

RNA was purified from lysate using a Maxwell RNA isolation robot and corresponding reagents (Promega) according to the provider instructions.

RNA quality was assessed and quantity measured by capillary electrophoresis on an Experion device (BioRad).

HTRA1 mRNA Quantification Using RNA Sequencing

Four hundred ng of total RNA was used to prepare mRNA sequencing libraries using the TruSeq™ Stranded mRNA library prep kit (Illumina 20020594). Libraries were sequenced on an Illumina HiSeq 4000 sequencer (2×50 bp). Base calling was performed with BCL to FASTQ file converter bcl2fastq v2.17.1.14 from Illumina (https://support.illumina.com/downloads.html). In order to estimate gene expression levels, paired-end RNASeq reads were mapped to the Macaca fascicularis genome (macFas5 from WashU) with STAR aligner version 2.5.2a using default mapping parameters (Dobin et al. 2013). Numbers of mapped reads for all RefSeq transcript variants of a gene (counts) were combined into a single value by using SAMTOOLS software (Li et al. 2009) and normalized as rpkms (number of mapped reads per kilobase transcript per million sequenced reads, Mortazavi et al. 2008).

HTRA1 Protein Quantification Using Capillary Electrophoresis Based Method

Proteins from different eye compartments were analyzed by capillary electrophoresis using a 12 −230 kDa separation matrix on a Peggy Sue device (Protein Simple, San Jose, Calif., USA), as described by the provider. Human recombinant 6His tagged-HTRA1 S328A (RD Biotech, France) samples (62.5-.0.12 ng/ml) were analyzed in parallel for quantification. HTRA1 protein was detected by using a rabbit anti-human HTRA1 antiserum SF1 (dilution 1:300), provided by Prof. Dr. Sascha Fauser. Cynomolgous RPE lysate, containing 5.5 ng/ml HTRA1s, was used to assess assay reproducibility.

Since preliminary experiments (not shown) excluded a matrix effect, samples were analyzed at the highest possible concentration enabling optimal quantification of HTRA1 suppression. Aqueous humor samples were used undiluted; while cleared homogenized vitreous samples were diluted 80% in RIPA buffer containing protease inhibitors. The concentrations of neural retina, RPE and choroid lysates was adjusted to 0.5 mg total protein/ml.

Throughout the study, intra-assay variation was 9.1% and inter-assay variation 21%. To improve comparability all samples from each tissue were analyzed in a single run. With the exception of the aqueous humor samples collected at different time points which were analyzed in a run distinct from the terminal samples.

Results 1: HTRA1 Protein Suppression in Different Tissue Compartments, Post Mortem, 32 Days after LNA Application:

The highest HTRA1 concentrations were found in the choroid lysates (PBS group: 28 ng/mg total protein sd=2.2), and the LNA treatment only caused a slight suppression within this anatomical region (16% decrease in the LNA group: 24 ng/mg total protein sd=3.5). The lowest HTRA1 concentrations measured were in the RPE lysats (PBS group: 5.2 ng/mg total protein sd=0.4), with significant I impact of the intervention (55% decrease, LNA group: 2.4 ng/mg total protein sd=0.2)

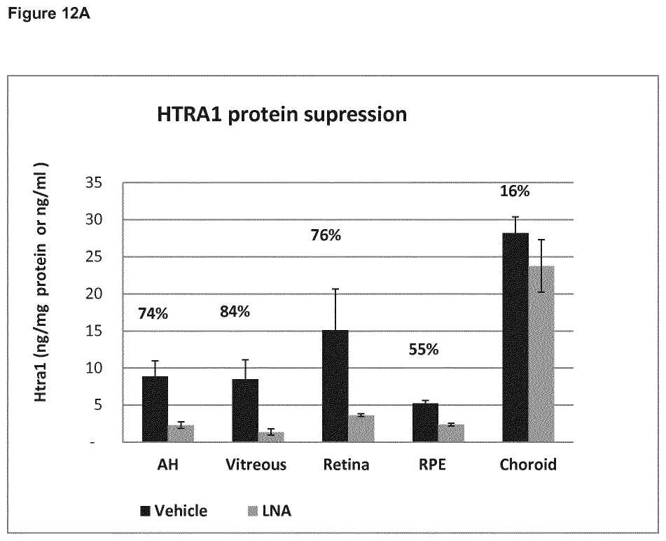

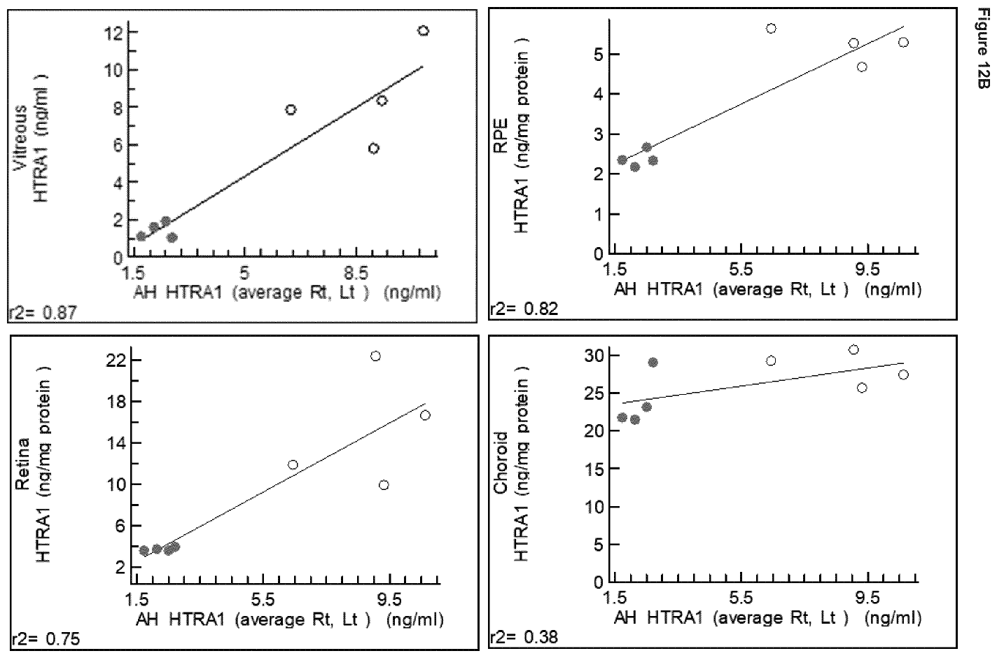

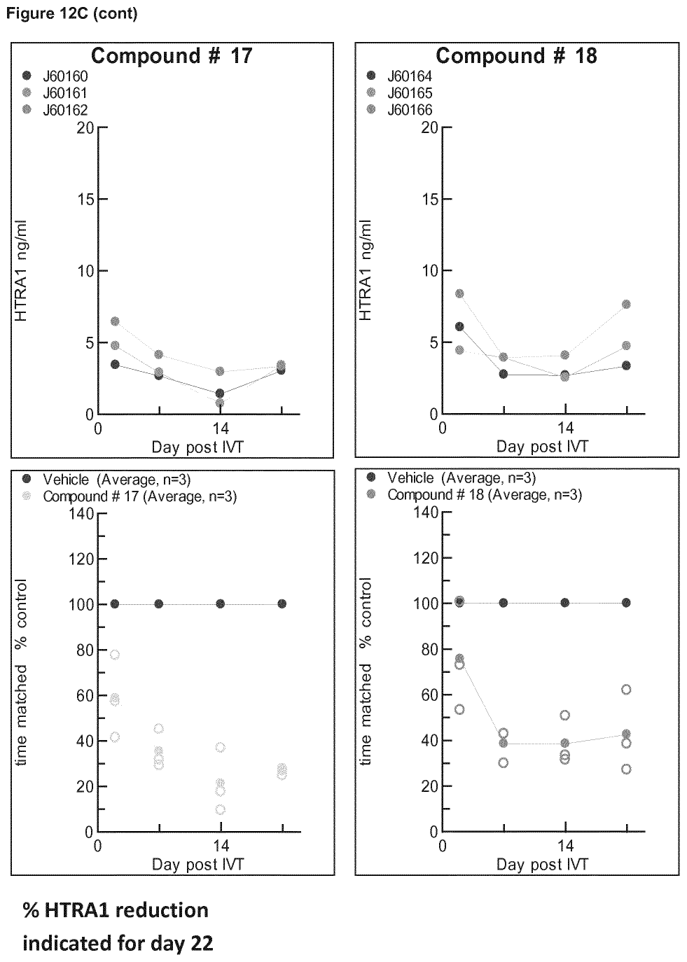

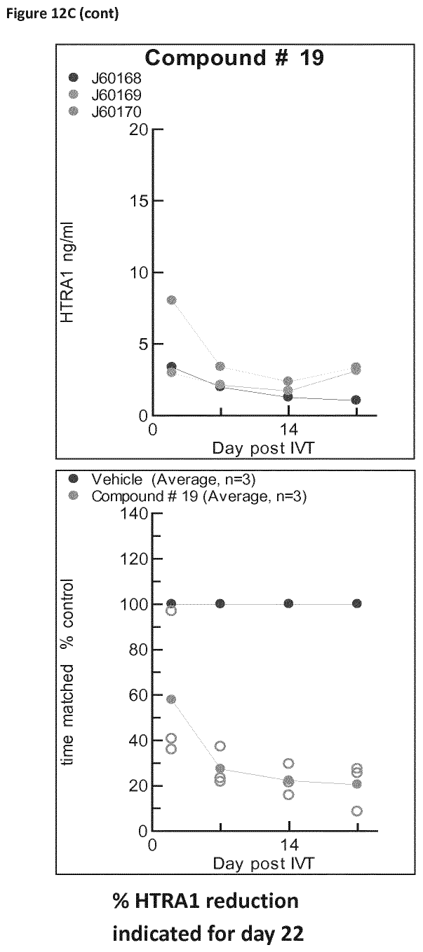

Highest impact of the intervention was observed in the neural retina; vitreous and aqueous humor (AH) with average HTRA1 levels 76; 84; and 74% lower than in vehicle treated animals, respectively. Group average suppression in the different compartments is shown in FIG. 12A.

Despite the dispersion of HTRA1 values determined the in the control group, Htra1 concentrations measured in vitreous, retina and RPE were in general accordance with levels measured in aqueous humor; suggesting potential usefulness as target engagement biomarker. Correlation between tissue HTRA1 level and aqueous humor levels illustrated in FIG. 12B

Results 2: Dynamic of HTRA1 Protein Concentration in Aqueous Humor

One baseline sample could not be measured due to insufficient sample availability and the animal (Vehicle treatment, Group1) was excluded from current analysis.

Baseline HTRA1 protein concentration in aqueous humor was heterogeneous (4.32 ng/ml standard deviation(sd) 0.98 ng/ml) with higher values in the treatment group. (Active treatment 3.65 ng/ml sd 0.18 ng/ml, vehicle 5.23 ng/ml sd 0.82 ng/ml). There was a trend for an increase in HTRA1 concentration in both groups at day3 post IVT, (Active treatment: n=2, +51% and +54%; Vehicle n=1, +42%). Therefore, effect of the intervention was analyzed after normalization to individual baseline values and to time matched vehicle group. The treatment had no effect on day 3 but aqueous humor HTRA1 concentration was reduced from day 11 on (52%), an effect reaching 66% suppression on day 36. Data is shown in FIG. 13.

Results 3: HTRA1 mRNA Suppression in Different Tissue Compartments, Post Mortem, 32 Days after LNA Application.

HTRA1 mRNA levels were measured by RNA sequencing in the retina, RPE, and choroid dissected from the left eye from eight animals (4 animals in the vehicle group and 4 animals in the LNA treated group). An average of 129,876,690 reads were generated per sample, with an average of 122,038,760 mapped reads per sample. One retina sample was excluded from the vehicle and LNA groups and one RPE sample was excluded from the vehicle group due to failure to pass quality control measures. In the remaining samples, HTRA1 mRNA levels were significantly and robustly reduced in the retina upon LNA treatment (80% decrease in the LNA group versus the vehicle group). A slightly lower (60%) yet significant decrease was observed in the RPE upon LNA treatment. No significant reduction was noted in the choroid (see FIG. 14).

### CONCLUSION

Single intravitreal injection of 100 μg HTRA1 LNA in Cynomolgous Monkeys led to 80% decreased HTRA1 mRNA expression in the neural retina and 60% reduction in the retinal pigment epithelium 36 day after exposure. Correspondingly HTRA1 protein reduction was 76% and 55% in respectively the neural retina and RPE, indicating a sustained drug activity and broad distribution and efficacy of the drug in the different eye compartments explored. As expected, due to the blood-retina barrier choroid was only minimally reached by the oligonucleotide. Consequently, no significant reduction of HTRA1 mRNA was observed in this tissue. Importantly, these data show a significant and prolonged impact in the RPE layer playing a pivotal role in AMD pathogenesis. The strong correlation between HTRA1 suppression in mRNA and protein levels indicates that local HTRA1 synthesis is the major source of the ocular protein pool and the proportion of blood-derived HTRA1 is negligible. HTRA1 protein levels in the essentially cell free vitreous and aqueous humors were reduced in an extent similar to the neural retina (r84% and 74%, respectively) indicating that protein concentrations in these biofluids can be used to assess the efficacy of the intervention in the back of the eye. Decrease in the aqueous humor could not be detected before day 11 illustrating a delayed onset of action and/or low HTRA1 protein turnover in this compartment.

### REFERENCES

Dobin et al. 2013, Bioinformatics 29: “STAR: ultrafast universal RNA-seq aligner”
Li et al. 2009, Bioinformatics 25: “The Sequence Alignment/Map format and SAMtools”
Mortazavi et al. 2008. Nature Methods 5: “Mapping and quantifying mammalian transcriptomes by RNA-Seq”

1. A method for determining the suitability of treatment of a subject for administration with an HTRA1 mRNA antagonist, said method comprising the steps of:
i) determining the level of HTRA1 in a sample of aqueous or vitreous humor obtained from the subject
ii) comparing the level of HTRA1 obtained from step i) with one or more reference samples or reference values;
to determine whether the subject is likely to be, or is suitable for, treatment of with the HTRA1 mRNA antagonist,
wherein the subject is suffering from or is at risk of developing an ocular disorder, such as macular degeneration.

2. The method according to claim 1, wherein the level of HTRA1 in the sample of aqueous humor or vitreous humor is determined by quantifying the level of HTRA1 protein in the sample.

3. The method according to claim 2, wherein the level of HTRA1 protein is determined using an immuno assay, such as using an HTRA1 specific antibody.

4. The method according to claim 3, wherein the level of HTRA1 protein is determined using mass spectrometry.

5. The method according to claim 1, wherein at least one of the reference sample or reference values is obtained from a control subject who is suffering from macular degeneration.

6. The method according to claim 1, wherein at least one of the reference samples or reference values is obtained from a control subject who is not suffering from macular degeneration.

7. The method according to claim 1, wherein the at least one of the reference samples is a value previously obtained from the same subject whose suitability of treatment is being assessed.

8. The method according to claim 1, wherein the reference values is derived from a dataset modeling the correlation between HTRA1 concentration in the retina and the HTRA1 concentration in the aqueous humor or vitreous humor.

9. The method according to claim 1, wherein the HTRA1 mRNA antagonist is selected from the group consisting of an antisense oligonucleotide targeting HTRA1 mRNA or pre-mRNA, an siRNA targeting HTRA1 mRNA, a ribozyme targeting HTRA1 mRNA or pre-mRNA.

10. The method according to claim 1 wherein the method is for determining whether the subject has an enhanced HTRA1 expression in the retina.

11. The method according to claim 1, wherein the subject is suffering from or is at risk of developing an ocular disorder selected from the group consisting of macular degenerative diseases such as age-related macular degeneration (AMD), including wet (exudative) AMD (including early, intermediate, and advanced wet AMD) and dry (nonexudative) AMD (including early, intermediate, and advanced dry AMD (e.g., geographic atrophy (GA)); diabetic retinopathy (DR) and other ischemia-related retinopathies; endophthalmitis; uveitis; choroidal neovascularization (CNV); retinopathy of prematurity (ROP); polypoidal choroidal vasculopathy (PCV); diabetic macular edema; pathological myopia; von Hippel-Lindau disease; histoplasmosis of the eye; Central Retinal Vein Occlusion (CRVO); corneal neovascularization; and retinal neovascularization.

12. The method according to claim 1, wherein the subject is suffering from or is at risk of developing age-related macular degeneration (AMD), such as AMD selected from the group consisting of wet (exudative) AMD (including early, intermediate, and advanced wet AMD), dry (non-exudative) AMD (including early, intermediate, and advanced dry AMD (e.g., geographic atrophy (GA)).

13. The method according to claim 1, wherein the method is for determining the suitable dose regimen for the administration with the HTRA1 mRNA antagonist.

14. The method according to claim 1, wherein the HTRA1 mRNA antagonist is an oligonucleotide which comprises a contiguous nucleotide region of 10-30 nucleotides which are fully complementary to a HTRA1 target nucleic acid sequence, such as SEQ ID NO 1 or SEQ ID NO 2.

15. The method according to claim 1, wherein the HTRA1 mRNA antagonist is or comprises an oligonucleotide which comprises a contiguous nucleotide sequence of at least 12 nucleotides in length which are at least 90% complementary to, such as fully complementary to SEQ ID NO 7 or SEQ ID NO 16.

16. The method according to claim 1, wherein the HTRA1 mRNA antagonist is or comprises an antisense oligonucleotide, such as an LNA gapmer oligonucleotide.

17. The method according to claim 1, wherein the HTRA1 mRNA antagonist is selected from the group consisting of
(SEQ ID NO 13, #13,1)
5′ TₛAₛTₛtₛtₛaₛcₛcₛtₛgₛgₛtₛTₛGₛTₛT 3′
(SEQ ID NO 15, #15,3)
5′ AₛtₛAₛTₛtₛtₛaₛcₛcₛtₛgₛgₛtₛTₛGₛTₛT 3′
(SEQ ID NO 17, #17)
5′ CₛTₛTₛCₛtₛtₛcₛtₛaₛtₛcₛtₛaₛ^{m}cₛgₛcₛAₛT 3',
(SEQ ID NO 18, #18)
5′ TₛAₛCₛTₛtₛtₛaₛaₛtₛaₛgₛcₛTₛCₛAₛA 3′;
and
(SEQ ID NO 19, #19)
5′ TₛTₛCₛtₛaₛtₛcₛtₛaₛ^{m}cₛgₛcₛaₛTₛTₛG 3′,
wherein a capital letter represents an beta-D oxy LNA nucleoside unit, a lower case letter represents a DNA nucleoside unit, subscript s represents a phosphorothioate internucleoside linkage, ^{m}c represents 5 methyl cytosine DNA nucleosides, and all LNA cytosines are 5-methyl cytosine; or a pharmaceutically acceptable salt thereof.

18. A method for treating a subject suffering from or at risk of developing macular degeneration, said method comprising performing the method according to claim 1, and administering to the subject an effective amount of an HTRA1 mRNA antagonist.

19. The method according to claim 1, wherein the HTRA1 mRNA antagonist is for administration via intra-vitral administration.

20.-23. (canceled)


SEQUENCE LISTING (19 sequences)

SEQ ID NO: 1 - protein, 480 aa, Homo sapiens
MQIPRAALLPLLLLLLAAPASAQLSRAGRSAPLAAGCPDRCEPARCPPQPEHCEGGRARDACGCCEVCGAPEGAACGLQEGPCGEGLQCVVPFGVPASATVRRRAQAGLCVCASSEPVCGSDANTYANLCQLRAASRRSERLHRPPVIVLQRGACGQGQEDPNSLRHKYNFIADVVEKIAPAVVHIELFRKLPFSKREVPVASGSGFIVSEDGLIVTNAHVVTNKHRVKVELKNGATYEAKIKDVDEKADIALIKIDHQGKLPVLLLGRSSELRPGEFVVAIGSPFSLQNTVTTGIVSTTQRGGKELGLRNSDMDYIQTDAIINYGNSGGPLVNLDGEVIGINTLKVTAGISFAIPSDKIKKFLTESHDRQAKGKAITKKKYIGIRMMSLTSSKAKELKDRHRDFPDVISGAYIIEVIPDTPAEAGGLKENDVIISINGQSVVSANDVSDVIKRESTLNMVVRRGNEDIMITVIPEEIDP

SEQ ID NO: 2 - protein, 462 aa, Macaca fascicularis
MQIPRAALLPLLLLLLLAAPASAQLSRAGRSPEHCEGGRARDACGCCEVCGAPEGAACGLQEGPCGEGLQCVVPFGVPASATVRRRAQAGLCVCASNEPVCGSDANTYANLCQLRAASRRSERLHRPPVIVLQRGACGQGQEDPNSLRHKYNFIADVVEKIAPAVVHIELFRKLPFSKREVPVASGSGFIVSEDGLIVTNAHVVTNKHRVKVELKNGATYEAKIKDVDEKADIALIKIDHQGKLPVLLLGRSSELRPGEFVVAIGSPFSLQNTVTTGIVSTTQRGGKELGLRNSDMDYIQTDAIINYGNSGGPLVNLDGEVIGINTLKVTAGISFAIPSDKIKKFLTESHDRQAKGKAITKKKYIGIRMMSLTSSKAKELKDRHRDFPDVISGAYIIEVIPDTPAEAGGLKENDVIISINGQSVVSANDVSDVIKRESTLNMVVRRGNEDIMITVIPEEIDP

SEQ ID NO: 3 - DNA, 2138 bp, Homo sapiens
caatgggctgggccgcgcggccgcgcgcactcgcacccgctgcccccgaggccctcctgcactctccccggcgccgctctccggccctcgccctgtccgccgccaccgccgccgccgccagagtcgccatgcagatcccgcgcgccgctcttctcccgctgctgctgctgctgctggcggcgcccgcctcggcgcagctgtcccgggccggccgctcggcgcctttggccgccgggtgcccagaccgctgcgagccggcgcgctgcccgccgcagccggagcactgcgagggcggccgggcccgggacgcgtgcggctgctgcgaggtgtgcggcgcgcccgagggcgccgcgtgcggcctgcaggagggcccgtgcggcgaggggctgcagtgcgtggtgcccttcggggtgccagcctcggccacggtgcggcggcgcgcgcaggccggcctctgtgtgtgcgccagcagcgagccggtgtgcggcagcgacgccaacacctacgccaacctgtgccagctgcgcgccgccagccgccgctccgagaggctgcaccggccgccggtcatcgtcctgcagcgcggagcctgcggccaagggcaggaagatcccaacagtttgcgccataaatataactttatcgcggacgtggtggagaagatcgcccctgccgtggttcatatcgaattgtttcgcaagcttccgttttctaaacgagaggtgccggtggctagtgggtctgggtttattgtgtcggaagatggactgatcgtgacaaatgcccacgtggtgaccaacaagcaccgggtcaaagttgagctgaagaacggtgccacttacgaagccaaaatcaaggatgtggatgagaaagcagacatcgcactcatcaaaattgaccaccagggcaagctgcctgtcctgctgcttggccgctcctcagagctgcggccgggagagttcgtggtcgccatcggaagcccgttttcccttcaaaacacagtcaccaccgggatcgtgagcaccacccagcgaggcggcaaagagctggggctccgcaactcagacatggactacatccagaccgacgccatcatcaactatggaaactcgggaggcccgttagtaaacctggacggtgaagtgattggaattaacactttgaaagtgacagctggaatctcctttgcaatcccatctgataagattaaaaagttcctcacggagtcccatgaccgacaggccaaaggaaaagccatcaccaagaagaagtatattggtatccgaatgatgtcactcacgtccagcaaagccaaagagctgaaggaccggcaccgggacttcccagacgtgatctcaggagcgtatataattgaagtaattcctgataccccagcagaagctggtggtctcaaggaaaacgacgtcataatcagcatcaatggacagtccgtggtctccgccaatgatgtcagcgacgtcattaaaagggaaagcaccctgaacatggtggtccgcaggggtaatgaagatatcatgatcacagtgattcccgaagaaattgacccataggcagaggcatgagctggacttcatgtttccctcaaagactctcccgtggatgacggatgaggactctgggctgctggaataggacactcaagacttttgactgccattttgtttgttcagtggagactccctggccaacagaatccttcttgatagtttgcaggcaaaacaaatgtaatgttgcagatccgcaggcagaagctctgcccttctgtatcctatgtatgcagtgtgctttttcttgccagcttgggccattcttgcttagacagtcagcatttgtctcctcctttaactgagtcatcatcttagtccaactaatgcagtcgatacaatgcgtagatagaagaagccccacgggagccaggatgggactggtcgtgtttgtgcttttctccaagtcagcacccaaaggtcaatgcacagagaccccgggtgggtgagcgctggcttctcaaacggccgaagttgcctcttttaggaatctctttggaattgggagcacgatgactctgagtttgagctattaaagtacttcttacacattgcaaaaaaaaaaaaaaaaaa

SEQ ID NO: 4 - DNA, 53384 bp, Homo sapiens
caatgggctgggccgcgcggccgcgcgcactcgcacccgctgcccccgaggccctcctgcactctccccggcgccgctctccggccctcgccctgtccgccgccaccgccgccgccgccagagtcgccatgcagatcccgcgcgccgctcttctcccgctgctgctgctgctgctggcggcgcccgcctcggcgcagctgtcccgggccggccgctcggcgcctttggccgccgggtgcccagaccgctgcgagccggcgcgctgcccgccgcagccggagcactgcgagggcggccgggcccgggacgcgtgcggctgctgcgaggtgtgcggcgcgcccgagggcgccgcgtgcggcctgcaggagggcccgtgcggcgaggggctgcagtgcgtggtgcccttcggggtgccagcctcggccacggtgcggcggcgcgcgcaggccggcctctgtgtgtgcgccagcagcgagccggtgtgcggcagcgacgccaacacctacgccaacctgtgccagctgcgcgccgccagccgccgctccgagaggctgcaccggccgccggtcatcgtcctgcagcgcggagcctgcggccaaggtactccgccgcgctcctgggcagctccccactctctccatcccagctcggacctgcttctgcgggactggtgggcaggttgaggggcagcgaagcgttgtggggtggccagggcaactctcggggacaggcaggtgggccccggggtggcggatttccgcgggctgcctcggaaccgagcttcgcgcccagcccggggccggttctgcgcccagacgatgccagtacgcccggcctgcactctggggctcgagacgccgggcgaccggccatggagtgccctgagggcaaccacacagcgcggggaccccaggacaaataagaggaatgggggcataaaggaaggagagaagttcaggactgggaattggcgcctcgcagagcggcttcaggaccacaagaagtcatttcggttgctttttcttctatttacgtcctccgtcccctttaaaattcactgctttgatcacgggaccgctcagtgaaaactgtatgtaactcttttggaaaggaacagtgtttgccggcccgccccggagtttctccaaaaagtctaccccgagcagggaacggtttggcaccgctctcgtttcggcggcgttgctgcctgtcttgctttcctcgttttgagccagccctacaaaaatgaaagtggctccttttgaataagctgaatcgggctttggatcacgaaatctgcagaggcggagaagggaccgggttagtgatgaggaagaagtctacccctctgttcctacagccgcacacaggacctgttctggcaggggagacggtggtgatgggggaaggagtggaatggagcaatgtctaactctctcgcgggaccttccggagagatgctcctcatcttcaggcagaggccatgtggaaaaataatatcgagttcagcagcggccagccccgcgttgtaggaaccagacagcggggcttggcagtgcgcttgggcgcagccgtgccgctgctgccggaccccagtgctgcctcctcaacacgggcagtgccaggagaggggcataggggagcacagtgcagagggactggtctagagtttactttataggaatatggttcggtgtgaccaactagggcttagcatagtttggcttacgtggacgggaagatgccagagccgaactgggtgaaattcgagattgcgtatttcaccaacacaggagcacagccctcgggaaactcagcctagtcaggcagtagagagttgtcccggagagaagtgatcctgcagactcgagaaggggcatgatgatagcacacgtctgttgagcacccagtctgtgtgccgggtgtgttacctctgtgacctcatttggtcaaacgaggaggcagttgctcctctctctctctttttttttcttaagagacagggtctccctctgtcgcccatgctggagtgtagtggtgtgatcatggctcactgcagcctccgacccctgggctcaatgattctcctgcttcagcctcccaagtggctgggactacaggcggatgccaccacacccagcttctcattcccgttttacagatagcggagctaaggttgaaaaacttgcccaaggtcattcagctggaatttaaacccagacagcctcattcagaggagtcagcccagcacttaactccaagggtgtgggagaggggtcaggtgctgtaaatttcctggtgggctggacgtgcatccccctcagagctgggaacagcatacacaaagcctaagacttgtttggaggtgaatagatcagtgtggctggggaacgttttgggagggcagcaggagtgagccaggctggtggcccagagtcccagggctgaagaggctggctgtgccccgtgccctgtgcgcagatgttcttgaactggagcaactcaaagcctagtgtagtgtagggctgacctagcagtggagtgcggaatgcatccagggtggagagtttagactactgcaataatctgggtgtgaggcgacaacattgaaaaagcatgtttttgtccaaaacaagccagctgttactggtctcgctgtttgtggtctcatcgcacggggtcctgagttgctggcaccatgcgagccgcctaatttattgctagtgaggcaagttgcttaacaagttttggagttggctgagtccctgtgtggaggaaaacaggtcccccattggccatcgggctcacagcgggcccccggtgtaccagtgaggggacagccacagagggataagcatggtggctttgaaaggagggagagacagagtgggtacaatgcttttcttatccctccctccttcttttgcaaatatttattgagctctgtagggtgtctgacaccgtttgcatgtttgtctgtctggcacatcggaggtacttggtacgagtggattagtgaatgaataaatgaatgaatgaagacaaacgggaggtgcttgcgatacacagccattctgtttttccttagtggaaggcactgctttgctgcgccccctctctggatctcacactccacccttgacttttcggaggtgtttccgaggacaggcgcctgggagccagcagacttcattcagtccaagccaggctccaggactcaacagctggtgcccacgggcaggtcacttgacgtcactgttaaatgaggtgaattggctgcctgctctggctggaagattggcgggagagtcactttagctgccatggacatgagccttttctaggggtgccacttgactagaggcctggagttggagcaagtcatacacggatctggagacagagctctcgaggcaggagcgggtgctgcgatttcaaatattataaggtggctttgtctggggcagagcatgccaggggatgagaggtagaaatgtcatcagatcaggggtccccagggaggtgactagcactttgggtcacagtagatctttggatagaggaacatgtcaccattcaaaggaaagcactttcatctgtaagctgtttattgaatagacctcagagaacatctctgctcaccgctctggaaatgaaggcaaatcatctatttcagaagtcaatgcactggcagggtttggatgggaaagtatacaattcagctagagaacaaagatctgtcatctccagctgtactggtcagatgattacaaaaaagaaaggaattgaaatactaatagggtactaataatgagggctaacatatatgttgtgcttattctatgccgggtgcatactaattcatttgatcctccggacagtcctatgagtgagtgctgtagtcttccctgggttacagctgggcagctaagtcacagagaagtaccttgctcaggactggtggtcccacacaactggatggagagcctcgttcataaccaccatgctgtgctgttgacagagcaacagagattttaaaccaaccccagctaagccccagctaatagctgaaataaacagggctccagatggctgtggcttagagatggaacaggacagatcacagccttcactctgcaggctcaggagcctgaagacaaggttgcctccagttgccgtcagtgcagccctcactaaagaaaagcaaaaagagccgagggactgtaggaaggctgtttccaagccagagatccagacaaactgctcttgaagagagaaagcccttccagattcccccatgtcccaaaagaccagccgggattccggacctctgctaaaacatggacaagaagccaggaacgagacctgaaacagacttcccaaacagcagaagcctcatccatttctcctgctagtacatcctccaggaaagcccaccctactccatgcagcagcccagacaagcttggaggtctgcaagctgcaggggtgcccagaaactccacccctggaggtttttaggatcgcctgctcctggtctcaccccagagcctctaaaggcagaggctgtatgtacatacctggtgaagaaccaagggcttagatggttgctttacttcttggagccctggaatgtttgtaaaatttacttttttttttgagacagtgtctcgttctgtcgcctacgctggagtgcagtggcgcgatctcggctcactgcaagctccacctcccgggttcatgccattctcctgccttagcctccagagtagctgggactacaggcacccgccaccacgcccagctaatttttttgtattttttggtagagacggggtttcaccgtgttagccaggatggtctccatctcctgaccttgtgatccgcccgctttggcctcccaaagtgctgggattacaggagtgagccactgcacccgtgccaaaatgtactttatttaggtgactctttcgtgggaacctcaaacaagcaatcattgctagctgagtgctgaccctgtactgagctctggggagacagggttgaataaaacaaagtcactgcccacaggtaacttatattcaatacaatgggggaaaatacaatcactgcttccctggggttgtatttttccattgttaaagtgggcagtttgctcgagagtcattttcactattggcaattcaaatacaccttttgtcagttaaaaaacaagtgtgccagggacctgagcttcatcttagggcagggtgggtggaaacatttgtgagtctccagcttttagtcacctgaaacttggaaacttggaggtcttttgagcagtttatgagtctctgcctgctctggtcggctgccttcttttattgctctgttggttttgctaaagagttaaaatattaaggcttcataaaattaggaagttaacaagctcaaaaaccaagtgtttgagttacttcattccactgagagagctgtaaatgggttgcattggaacttaaaataactgcattgagtaagtgatggtggcgggcaccatgagctaactgtggtcagaagcctgatggcctccgctttggggctggattctccgtttggagctgtgtgatcctggatgagtttcatgccttggattcagaaatcagactttccatgagcttatatttcaagtgaataaatagctctggtcaggcttaatttgaagaagaagtaagcttggcagtgggtgagggttccttggaaggccaactggggcggaggggctgagggcaagcggctctggcccttcctggggtgttacctgaccaggtaacagctccctcgacctctcggagcctcggcagtgaggggattgggccagttgatctctgaggctccttttaactagaatggtctgggatttttctaagaaaacaagtctttgaggaggttgtggtcacctcattcctaatttaaagcctggggaggcttccttatgagctacttctttttcctaaattattgatggttaaagccaaggctggcatcgaatagatgtgatccatcttgagcctggttgctttgtgtttcagctttgtactggctgctgaagtccccgggagaccacaggggtgacatgttcatctccaagagatgagcttccacgagactcataccccttgctccttccctggggctccaaggcctttgggtcatctgaagtgagatacccttgtgtcatttcatcttttccttctccaccttctctgccgttaaaaaaaaaagaagaaagagaaaaatcctattaatagagaaaccgagaagtgtagccattctgaatgtgtttccaaaaggctcctggaagtggcatggaagttacagtgattcagcactacttggtgacgtgtgcctagaaccacagggggacattagccaggacaacacgcctcaggacagaagtaagtggctgcgaagaggcatgtccatcactgccggaaagatgcagagttcagtttttggagtcagtgctgagagttccatttctaaattcattcagagcatttatttaacacctactgtgtgctcagaagtgtatcaggtatggggactcagaggtaagggctggtggcccctgatctcaaggtactcgtggtagatagtatgatgctcagcttaagggctgggcttctgaagtcggattgccattttctggatgtgtggtgtttcttgggtgacttcatctctaagtctcagtttccccatcagtaagataagagaagtaatagcagatacatacgtagctcttagggcattgcagaatggaaggacctccttatatgaaacgcaaagcactgtgcctgatgcattgctagaactcaggcaatattagcgtgttgtcattgtcatcatcatcatcatcatcatcatcatcatcatcatcatcttcaaggcactgacaaaggagtcagctgtgtgggaggagtgctgggacactcttgtctccctggggatgaggtgggtgggtgggttaggaaatcttcacagagaaggagggtgatgtgagacttctgtccgggagctgactcggaatttgccatctaatatgttggaaaaggttctctgggcagaggtatccaaagtcactttgcctgtcaccctttgaggtcccagttgttgcctatatcatgtgaccagtgtgtggcttctcttgaattaagagctgcatgtctggactgcctgggattttacagatgtcatctcgttaactctccctggagcttgtgacacccaggagatggcagtttatagaagccctggcaccttcttgaatgatgcttggtttggtttctatgcactgggaattcctcacaaggaaagatttgtcacatcttaaggaaggaaaaaaaggcaaatttgggagtccatggataccctattattttagattccaggacaaattgtcgaataagcacgtttcataaaaacaatcctccgcagcatcccgtgacagcagctggtccctcgccacaggataattatgtctccttgtgcacacaaaagtctccgagggcatattgttgtggctggagtttctgataatttccaaattgaacaacctcagtcctaatgagtcagaggcttgtgcaatattttcaaacctcaggaacatctttttcattagttgtgcaataaagatggtaggcctatctctgtgatgagctgtttttttttctcaaagtttgatgagattcgccgtagaattccttctcacatagtcttgggcaagattttacccgatcttccaacacatgagtcatctcatatcctgtgactaagaagagctgtctctttggtgccagttttctaagtgcagtcaccacttgatggagacggatggacacagttgggattgcccaggcagatgggcaatcttgccagctagacataggggagggaagcctcaatgttcagcggtcacatctgcttttctgtggcacagagtgagctatacaggaatattgtattctccaggacagttagggcagtgggaaatgtcatcaaacagaacagtgacccaaagagccactgccactgggtgctctgtgggagctgggcactgtgctcattgtgttatgggccttgctttgttcttaccttgtagccacccagagaggcagggcattatccttgcttcctagctgaggccacagaagaggctcctagaggttagctgtaacttgtccaaggccagccagtgcaaggaggcagagccaggatttgagcccatgtctgtttcactcccaaactattcttcagatttctttaagtcaagtgttatttagaaatgttttgtttattcatcaaatatttggtgggtgtttccagctatctttctgttattaatttctagtttaattctattgtgggctgagaatatattttgtatgatttctattctattacgtttgttagggtgtattttctggtctagaatgtggtctgtcttggtgagtgttccctgtgtgcttgagaggaatgtgtgttctgtcattgttgaatggagtgttctataaatgtcacttaggtctagtggattgatagtgcggttcaggtcaactgtatccttcctgattttctgcctactgatctatcaattcctgaaagagaagtgttgacgtctcctgagtctattctgaaacactgaattgcggtctccatgatgaaccactagagttagaaaacctgggtcctagccccatttgggcctttgggatgactcccttctgcctcagtttcctcatctacaacagggggacaatgatgctgcctaggagacatcagcaggatactgtgaaagtccagtggcataaggggtatggaggagcttcgtcaactcctaaagcttcagtgctaggaatcctaaagcattgaaatccaaagatataaggaatatgaaggagttttgtcaattcctaatgcttcagtgctaggaatcctaaagcattaaagtccaatgatataaggaatatgaaggagctttgtcaactcctaaagcttcaatgctaggaatcctaaagcattgaagtccagtgatataaggaatatgaaggagttttatcaactcccaatgcttcagtgctaggaatcctaaagcactgaagtccaatgatacaaggaatatgaaggagctttgtcaactcctaaagcttcagtgctttaggagtcctaaagcattgaagctgtaagagattaggacctctagttggcaattccagactcttccaggactcctgatagagccaacaccaagaatagtgaagccagaaggatggaaatagtaaaatgcctcctgggtgtcaaagcatgggtctcctctgggcatgttctcttgtcctactgagacatgatagctcttggccaaagtgactgaacttgaccctctgtttcaggaaggccaaatgcagggttcactaccatcatgtccaagggcagatgcgttggtccagaacatcagcatcccaatcattataccaagcaaacagccgtctctgcctgcaccgtggagagcacacgctcctcctggggtggcctgcatcctgtgttcttctcaggccgactttctgtttaatgtttgctggtcaggaaatggcctgagctgaggttcttcagatcccagtctgacctttctccaccagcatttgtggctctgaaaaatatagcccagtgtggtttagccccactggatgaaacccagtaggaaaagtctgataatagcagaagacgcacaggaggaagagtgaggatttgagagcatctgggaaggaccatgtgcctggatatcgttctgtctgtgggattctgtgacacttgtcatttacagtctgttcccatggaattctcatcattggccaaacatatagtccttctgtcctctgaaaaatatcattctgctccgacctttcacacccatctctgaccacatcaactccctgtttgcatgcatcttgtggatgaaggacaccactttacctgtaaagacactggtggcttcccaaagccaccaactgacttgtagagaagacagaatcccagagtatgaaacctgagggtgaagggtcctggcaggtcctagagctcaacccttcacttcacaggtggggaaactgagggagccaatgggaacatgactctcacaagctgcacagctcatctgtaggggccagtgtggagtctgtttgtcctgagacccagggctgagcctttgagccctccgcatctcagccgcatcctcctgttggagcagttaggtgtttgggagaggccacggtccatgctcatggttttcctgtaaggctggagaaacaggccttgttcccttagtctctctaatcaaaatgaggttgcagaaaacccttctccctacttctccctaaaataatttccttgggttagaagatgactaaaagactattcatccgatgactgatgtctcccttcaagagttataagcacatataaatgcctttgaatggtaattataataattttgctgaagggaaaatatcagtataaatatcatggtggacacatggaatgaggactgagatgctttcatgtcttttcagctgtggttagattttctttaagcagaatatacaagtttttcctctcctagcataaggactctttttttttgtatcttttctctctactttttagacatgatggaaaatgcatttatacatttgatgacatattgtactatctcagttgtttaaaattataaatgtaatttaatcatatgaaaaattaagaaaagaagattcatatttcaccatcatctccccagaaatatcatttctttattactattattattattattattattattattattattattattattttgagacagggtcttgctccatcacccaggctggagtaaggggcacgatcttgactccctgcaacctccacctcccaggttcaagcagttctcatgcctcagcctcctcagtagctgggattacaggcctgcaccaccacacccagctaccttttatatttttaagtagagacagtttcgccatgttggccagactggtctcgaactcctggcctcaagtgattggcctgcttcagcctcccaaagtgtggggattacaggcatgagctaccatgcctggcctaattccatcatttctgtcccaagtgttgccaccgtttggttaactgttcccctgttcacatccatttgggccaaggttgcaatgttaaacaatcctgagatggacattttcatgtttatggctatttctgtatctagggtcattctcttaggagaggtactaaggagtacaaaaactgggaagaaggatatggaatttttatggatctggtataaattgccaaattattttccagaagggttgtagccatatttgttgccatcagctctagaatttcaacctcgtaagtcactgaaagaaattctcccaaaatcaatccttcaggaataatggaagaagatggtgccaaaccccagccattctgctcactgttagattccttttttggtcttacaggttacttttattctcaggttgatggctcttagagttgagcaatgtttggggtagaataacgagcacttttaaaacttggttctacctggggagggggtgagttgtgatcacagacagtctcacctgggaggggcttgggtgtttgtcggcttgtccttctaacactcgtgtctcaggcgagcagcctgggaccagtgaggtgacctgaaggctggaggtcacaagctaagaggcgacagagaacccaggtctcaggaagcccagcccagagctcgctgcactgagcctctcggatgccagctctgtccaggatgcgggaggaggccagactgatttggtctgttttgaaaagtgatgaaaatatttattcaaatgttttgtactcataggcagaagtataacaggagctgcatatacaaaattattttctagtagtcacattaaaaaagtaaaaagaaagaacacgattatttttctttttaaaacagctttattgagagataatttacatactataaaatttacccctttaaagtgtacaatttgctgttcttatatattcacaatcatgcacgtatcactaccagctccaggacactttcatcaccgtaaaaagaaaccccgtatccattagtagccaccccatacttctcctctgcccagccctaggaaaccaccggttcattttctatttctatgaatttgcttattctggacatttcatataaatggaatcaaagaatacgtaacgggcttctgtctcttagcataatgttttcaaggttgtccacattgtagcatggatcattatttcattccattttatgattaaaaatatgccttttaagggatacagggagaccagacgtctattttatctcccctccctgatggggaatcctaatttcagcctggaaagtcactgcgaaagtctaaactgcagaggtgatactgtttccactggaagaaactgtagcacctgactcaggaagccagcattaaaaccaagaatattctatatggatggggattacgcactgaaaggaaaacatgaggaaatgcacttttcagatttattagatcatagaacttttttggagctggaaaggatgtcggaaaccgtctagcctaccccctcatcttaccactgaggtaactgaggcccaggaaggggaagtggcttgttttgggtccgggaccactcttcatttcttatttgagccaaagcttccttctggcgtctgtctctgtttcacaagttcccctcgcatgggggctgggtactgcttggaagaactggcttcttccttgatacaggggctcgttcaccatcacctccctccctcacgtctcttctgcctctctgcagcctcaggccctcctcctgcaccaggggggcagactcaacccgggtgggcactgcctcccagtccgtggccagaggctggagggctagggagactgaacagccccggcagctccagacataacaacctatgttgaggagtcagggcaggaagcgaacccagctgagaaatctgcgaaggtcaggaccagagccagacgcttatcaagagcaaagttaatggtttttgtgaaccgagcagtcagctgtttccccgaagataataatagacacatcatgttgggcattcaggaggcatctgaaaaaaaaaatgtgcagtggaattgattggaagcttttccctaatgcataaaataggccagaaaagactatcaaatgtaacagcaccgatcaaacccaatagatcaagcaaggactgaaaaacacaattttttttttctttgccagtgagtctgaaaagtgattttcaatgacaggcgcctttaaacatagacaacataaacaacaacatagttgttctggaagaggcatcttttcccagtaaagccaaagatgcagatctaggctgtgcttgtgactgacagcacagtgaggggttcacagccagctggccaggtgccccccgaaagcacatttcgaatctactctatttgagagagactgccttagccttgtttgggtaagtcttcctccttcacttcacctgccacagacttttccaggcaccatctgctgcagtcttggcccagcccctgcaacagttactgctcaaggcacccgggacatgcaggacgggggagcagcctgaggtctggcgtccggcgagcttttcccacttggagccgtctgggagactgtcccggaaagagaggggctgccaacacttggaagtgccaatgtgtgctgcaagtcgaggccaggctcccggctcccccgcctcttcctccttgattcattaaaaggaaagaaagaggccacacgaaactctcctgaatttcatttctttgtttctatgcaaaagacagagcgtggtcattcatcattcaaattttagcctttttaaacaaataataattcctgcttgtgaattcagtgtattttaacaagagtaggtctgagggccgttggccgtgtctttccttagatttgcagacagcggccctgatggtgcatagggtttcaggtttcctttagacctcagctggctgcctgggccaccacttagcaatgccattgtccttcctgtgcattttctttgcagaattcgaggaaatccagtcgcacaggcccctctgtgcccatgtccccggcgccctggaatgtgcagtaccagcagcagcgattagaatgggggtctggtttcccggaatgtgcaaggtctggcttctgtttctgctgcctccatgccccagaccagtgctgggccgggctctgggctggagccgtggctgacaagtttccttggaatttaatggagcgggccagacagcatgcagccactcaaactgaaaacctgggaaagaaatgagtgttgtggggcagctttgctgcattcactgggtcatatatgcttctttttcttttcctcaggcaacccctcttgcagacaggaggccccctcccctttcgcttcatgcctcactggccattaggaaccttttaaaactgatttctctcctgaccctcagagagaacatagtccaagttccctggaggaggaggaagcgctctgtgtttctctgcagttcacggctcagttaaatgcagcctacgtgctgtctttccccactcctctgcctgctcccgttgtgcttctcatgatcattctcaaattcagcgagaaacctcacaaagggagcttttcttagggaagagtcatccttggcctcccgaatgtggaccagcccctctccccagctgcacagcatcaggttagttaaccacctgcctccatctgggtcctgtctggacaggcctactcacacctgctgcaggcatccaacttgccctcaggtgcctgtggctcgtccagaggggtggagcccacattccagtcctgacaggtaaagttcagtggcggggaccctgcatttagtgtaaagatcaatattccaggtcctctcttcctgccacccagcgactggccgtttgcaggcactcggtcccagttgtcctgggcctgcagcccttgcattctctctgctttgtctctgctattgcacccctgccccatcagaaatgcaggtgggggggccttccgctgggacagtgagagactgggtagtaaggggagcgctagagggatggttgcgcttgcatccagccctgactgcattcgctctcccccgcctctctgtgaaggtgctgagctgtgagtggaaccaagtggatgagagtggccttgggcacctgccgataaatttcccggtgtgtcttctcctcctgggagtcccatctggatttgggtctggatttatttattcagcaagtagcctctttatagttactttttttttttttttttttttgagatggagtttcactttgtcacccaggctggagtgcactggcgcaatcttggctcactgcaagctccgccttccaggttcacgccattctcctgcctcagcctcccgagtagctgggaccacaggtgcctgccaccatgcctggctaattttttgtatttttagtagagactgggtttcactgtgttagccaggacggtctcgatctcctgacctcatgatctgcccaccttggcctcccaaagcgctgggattacaggtgtgagccaccatgcccggcctgtagttacttttaatttagccatgctcggggctgaaggggatgccaaagaaatataagatgagcccctcagacggctaaagatgaagatgaggcctccagtatgtacctcccacatacaccccaggaaattctgggtgtcactggattctggacctcccaaaagctgctggcacctggaggatggggccccgaggctggacctcactcctgctgggttgctggactgggaaagtactgatggcagctgaggagtgtgtcccagacttcactgagccattcccaaagattattccaagttctcctgacactgcactggaggcctgctgtgctggccttctttatttacagtttctgactggtgtctagcagccctgccagagagagcggcagtgtgtctgcaggcgaccaggagaaatgtctcaggctttagagcaggactttgagcacatagctgtgggggcccagcaggctgtctcctgcacggttacttctccttgtcctttcatggtcgagaggttgctgcctggcccttcaagtgaggatgggacatgctatccattggccttaatttccaacctctgcatgatgcattttatgctcctgcctttgaaagaacttttattttcttgtcatttatgcccagaccccacatggcagaaggaagggaggctgggacaggggaggcggataagctgccgctgacagacctgcccagtttcttagctcatcccggcctccatcctggtgagcagacactggcccaatccagccatatttttggctgagtttctgtcttcacatctcatccttaaccctgaatcctggccatagttggtactgggttgtattcttatttgtaatctttaaagtaggaatacctttgctggtatttaaagtggaagaaatcaggtgaagaatcacaagtgatttgcaaactggaagagacattagaatgtaaatgtgaggaagcgtcagcatgaggggcttgcctgggctgcacagcttgccttggctggagtatgcactgttctggcattgcagagaggatgggtaccttgcctccctgcaggtgggggactgtatcagcccccgcagactgctcctgggctcctgagtttgacagattttttttttttttttttgagacggactctcactctgttgcccaggctggagtgcagtggtgcgatctcggctcactgcaagctccacctcctgggttcacgccattctcctgcctcagcctcccgagtagctgggactacaggcgcctgccaccacgcctggctaattttttgtatttttagtagagacagggtttcaccgtgttagccaggatggtctcgatttcctgacctcatgatctgcctgccttggcctcccaaagtgctgggattacaggcttgagccactcgcccggctgagtttgaccagattaaggcagcatctccagtggcacctgagcagctcctgagatgcttttctgtgctaaatctggatttggggtattaaatcaaatgaatttgaaatgcaggcacagctggccccatgggcatggacctgtgcagtcacaccttgccccgtgttcagaagggtgctgtgcctgttttaatgctctgctgttgctctcttgagattcttaataatttttgaacaaagggccccacatactcattttgtactgggtactgcatattatgtagctagtcttgaatctaggacagtgcattaaaatgccattgattggatcaatctgctcttgcaactgatttgaattttgggaacatgctgtttcctgtgaataaaggaggattcatttcttttccctcgaatacactgcgttctgttttccaaattagctctacgtatcaactcagctgagaaattggaagcggggattgttctggctggaagggaaggttagattgttaatcctgcatcctggccctgatctcaccgagtgtgaagcatgttcccacaatggtgtgggctgcggggggctggaggctggctgagaaggtggggaccaaggagggaggctagcctgggagccagacagatggggttaggctcttgcttttgccactcgccagctctgaggcttagggcaacatgatttaattctctgatccttgtttttttcatctttctgtagactggtgatgagatgcaccctgcaggcttgcaggcttgcaggagtaattaaaggtaatatttgtgcctattattgggcttgacatatagtagatgctctacaataaatagatcctattattcttattgataatattattttattgctaacattgaaggttgggtgggatttgactagctggaggcgaggagaatgagatcatccaggccggaaggaaaagagacatgaatgcagggggatggggtggagcactttggaggtgtggggagaggtctgcagggtgggagttgtgcattaaggagtcgtggggagagtggaggaatcagtgccacatggtgaatgagaggggatcgtgggcccgaggagatggcgatggctgcggggatcctgcaggaagtttatgtgccccaaagtggcattatcagttagggggagacactgaagacagaggtgaggcctgcctgaattagcgtagagtgggattcttggaagcttcagaagcttgagaagagccacttggaggtgttgaaatgcacctgggagggacgtggggacccagctctgggctgagagctgggagacggaaacgcaggtgaccttggccttgaagatggggcatgatatttagtgctttatgtgcaatctcacctaggactcccaagccctttggagtaggtgatattagctccgtgttacagaaagggagactgaggctgaagcagggacattcatgatctgaagtcacacagctgtacggggcagaagtgggcatggaggcattaacttagagccgaaaggtgtgacctttcttagtgtggctggccccacggggaacgtgtgtgggttggagtacaacttggtgttcctacccatcccagatgctctgcgtttgtgaaccccagttgccacatcagggcgggcgagggcaggaagctctgcagggagaagggacaagggacagagccaagaacaggggcagtgccccagggtcctgcaggggcaatgaagggggttggcacacctgggttagttgctggccagtgtggggagagagctggcctgggagtctaatgggaatgccagggaaagctgccttggtcccctaaagtgaagcccccatgctggccatggagtgttggtgattgagggtccctgctagttgtctggccgaggcagcatgtcctataggcatagctctggtgtcctgctggcgtggcgtgagtgcccctcatgctgggagccagccctgtgctctggagggaggtggtgggaggacaagggacagtgggacctgccacctgagcaggaattggcaccttctcccactggcaggtccaggttttatggaatctgaaacttgtacaattcagtataccctcttcaagaaaaacacccctcaaaattatgaatataacattaggtatgaaactattattgatatagattgaaaaaagaaaatgcccaaaatgacaaacttcagaaaatagacaaatactgcaaacatcacaaaatcagaaaaataagattaaaaaaagctaactgctgaacactccgtcatcttgaaaatgcccctctctcctcctctattttttggctgtgaactctttgctcaccttttcatgtgacaatgcttttgtaatatttcctacagagaaaatagaataatttattattacttttattgtttttggattattattatgatcaattcaatatttttctgctacccacacactcactgtcttctgtccaacctctggcctgcaccaggggaaccagcagtttcccctgccatagggtgtccctggagaccacacatatagcaggatagatatagcaatttaactagacacagaagggacttcaaagccacaaatatatctcatttaacctgaacaaaatgattatccagttttacttttcccttagcctcttcccccaaatgctggcagccaccctgatgggatagatgtgtgacagagggcaagagaccgtggccccaaccagctgcagcttcactctttcatttctgtatactctctacaagctgtgatgatagcactttgctagggcccctcacagggcagatggagggctccacgctgaagctttgtggatgtttgctgtctatccacctctgctccttgtgcctatgcagggattcaggcccaaccactgcagagagcccaagagcatcaggcagaggttcccaaactgtcatgattggtggcacctttagtagttgatacggtttggttgtgtcctcacccaaatctcatcttgaattcccacatgttgtgggagggacccggttggtggtaattgaatcatgggggcagatctttcccgcactgttctcatgatagtgaataagtctcccaagatctgttggctttataaaggggagtttccctgcacaagctctctctctgactgctgccatccatgtaagacatgacatgctcctccttgcctcccaccatgattgtgaggcttccccagccacgtggaactggaagtccaataaaacctccttcttttgtaaatcacccagtctcaggtatgtctttatcagcagtgtgaaaatggactaatacagtagtgcagtcattttttcatggtccccagtaaggccaaaaaatacccaacagttccatttatcaattagtggaggccaaacaatttgataagtatttgtgtccctataacacagtggtcattaaaaaaagacattttaatttcattattcaataagcatgattacttatgaatgggatatgtgcacctgttgggtgtcacatgacctttcaaatcttggagtcagattggacaccaccatgcccatttccagttcaactctgatttttgtgtggtacatgctttttatcacagtgactgccagaaatccaacttcatatggaatcatgaaaagggatgtagtgtgatctgatttcaaaactatgatcaatctagagctagtttacaaggtgtctaacagtgatcaagtatcactgtatttccctagaaaacctgaaatatcgatgaattttctgtggcactctggggtcccttggggcacactatgggaaccatgggattaggaccataaggatatgattttggcttcttcctgcctcagatctaatctttacctggcatttttgccttaaagatgaaagaagcatacattttgatgtatttaaagcacatattcggccaggtgcggtggctcacacctgtagtcccagcactttgggtggctgaggcaggcagatcacaaggttggaagtttgagaccagcctgaccaacatggtgaaaccccatctctactaaaaatacaaaaaatagctgggtgtggtggcatgtgcctgtaatcccagctactcaggaggctgaggcaggagaatcacttgaacccaggaggcagaggttgcagtgaaccaagattgcaccactgcactccagcctgggctacagagcaagactctgtctcaaaaaaaaaaaaaaaaaaaaaaaagcacatattcattttgtgcttattcttttgagagaaacacagataaaagcctatcctttaattcatactccccatactgtgattttcatttttactgcaacaaattttgttcagtgtgataatgaatgtcaaacacttaatgccttgctcttttcagtaacatgacatattggagaataatgactgaagcttatctacactgcctacgtctgttttcttccaccttgaaagaagttgttgaaagtaattaagaagtattatgtgtaaaactccagggatgatgtgcttcaaggaagcaacatttatgaagttgtgtgcttgactagtagtttataaagaggaaagacgaatcatttattgtcttgggattgaatcttggcaatttttaaactataaagttacaggaaatgttggctgctcttaatgggccatttgttgtgttaaaaatcagtaatgagaaatatttactaggtaagtggaaagatccatctctataaattgttgtaacttaccattttacaaatcttagttactcagtttttctgcttaaaaatgaaatcatgtagcactgtataagtcattcagttttttattttggagaattactctggattgtctaggctctgtgctctccacatatatttttgaaatagtttgtgaatttctacaaaaactcctgctcagaattttcactgagagtatgcttaatctatgggttaatttgtgagaaattgatagcttaacaatagtgaatcttctgatctacaagtgtggtatttctctccatttatttaggtcttctttattttgatagcgttttgtagctttcaatgtacagatcttgcaaatatcttgttaaatatttccctaattacttgatatttatttttgatgctgttatagttatattttaaaaattttgattccaattgttgctaatacatagaaatgaaattatttattgacctcttatcctgtgacattgataaacgcagtcatatattcgtagatttctagaatttttctatatagactatcatatatatcatctgcaaataaagacggttttacattttcctttccaatctctatgccttttgtttctttctcatgcctcattgtgtggtccattactgaacggcagccagttccagctttctgttcaattaaggagcaggtaaaatggccaggccttgacctttcagggggcttcccgtcctcattgccttctgctgcctcagttctggcttaacagaacagtgtggggaggaggcatggtccttacctactagggcgttacttggccttcttcaggttggttgcttcgtcaggtttaagagctcacctgggctgcagttcaggctaggttatctgctgacctggccctgtctcccttctgtagtgtctgtggggtacccttgtaagctagggagaagagacacacgtgaaggccagaaaaaacagcctgccacacagcttccctggatcataccttcgcagtgacatgacgacgtcgttaggaggcgccgaggtggctgagtgggtctccagacacctccctttacctctctgctgtgccactgatgtgtgacttgcttacacctatgcagagctgccactgagcagcactgtggccagtcctttggattttcttctttctaaattgtatgccgtggcttgatcaagcatttcatatacagtagatcatgaaatcagcatagaaaacacattgaggtaggtggtgttaccacattttatggatgagaggctaacacttggaggagtcaggtaacatgtccaaggccacacagctagtgagtaccctgctgagggtcacactctggtccatctgaggccagagcctgtgccagccttctcctcatgctgatagacgaggaaacagaaagaaggagcagtggacgcccccaccctctgtcccctgaaccccttggagagtaggcagtggcagagccagcctgggcccatctatgggaattctccatcgggattgactcctctggaaggaagacagttgacccacagttgagatcacagcagatgggccagccagggtgtctgtagaccatcaggcagtggccactccatgtagtttaatggacaagcccttttaatggaacaggaatctaacactgaaccaagctgcttttagacacacttttattcctcactctgaaatggcgtttggacaagccaaatatttcttcttctttcagttgacattttgtccatctttgaactgttagttgatgcttcttctgtttagttattcctgttctattttcctgttgccactagtccacccagggatggtaagaatggaagtcaatggttgctttttcatctgagatgcaccacgaaggcttgtcagtcagccttgtcatatggtctgtgctcccactgctccttctttctgtttcctcatctgcagaatttggagagtcctggacctgatctcaaatttcacatgttatttatcttcctgcagcacgctggggagaggaagagacagggacatagaaggttggagctggaacagacttcacatctcattccagaggcatttggtccatcttacagatgaggaaatggaggctgctcagtggactgaggctggaactgggccttccagtggccaggccagatcctccttgatctcccttgttgctttcctggtgggaagaccctggaaccactttatgtgactgtgtgagaagggaactgcctctcattttacccagcaaaatccaccttcaatccatcttcatttttgcccctggtgtgggcaaattctcccatacctaattcaggaagccagaaagaggaagtgagttaatgatccttagtgggaaggcgctggtaatggtccttcttgtgagagtttctgaaacaccacgctgtctctgtgttctggcctggctggagttaaacctcttcttggcctttccccaggaagctggtctgaggaagcccagatgcgtttgtttacagctgtctggtgacattcgccaggctctgttttcagaaggaacatttccattcccttatttacacctcccattggagtgctcggggggacacaccaattatttgcaactacctggaaacctaggagggtagcagatctgtaggaggccagtgttgaagtgagaagctgtagatctggtgacactgtgggcttgggagggcttgcccagatctgttacttatactctctattaagaaacttcagtgtccatggagaagttatttaaagtctgcgagcctcagtttccccatatataatatgggaaggatacctgattttcctattccacatgaaggtagaaaaaattaaattaaggcagccaatgaaagggttttgaaagcaaaaataataatatgatactgttctgaatttgttaaattattcttccaagtagttgcagatctttttctgtaccttagaaaaaaaccatgctatgtaaaaggagatgattccaatctttaaataaagcaactcagaggtcaggggctaggacagaaaacggccctttgttcacagaagcgctctcacttccaagaaagcaagcgtgggagaggcaggtggtcctcccgatgtccctgtgccccatggtgtcaagctgggttactatggcccttcgtgacccagtgcagcagggatgtgggaaccagtgggtgtgaagctgtgacgggtcacaagagggctgggacgtctcacagcttttacttatagcctagagcctggggaagggttgccactctagtgatgagagaggcgtgtgtgtgtgtgtgtgtatgcgtctgtatgtatgtgtgcatttgcatgtatatatgtgtgactgtatgtatgtgcacatctgtgagtatatgaatgtgtgtggaagtgtgtataggtgtttatgtgacagtttgtgtgtaaatgtgggtgtatgtgtgggtgtgtttatgcatgtacatctgtgggtgtgtatgcatagtgtgtatgtgtgagtttgtgtgtgtgtgtgcatttgcatctctgtgtatatatgcatgtgtgttaggggcaggcacacaggcctgttggtaaatgagacacaaaatacctacaaaatacaaaatgtgagacaggaaatacaagccccagttactcatttttcagtgcaacagacataagattaccatgtgaaattgctatgaaagtttccgaaagcttcctgtcaattcgtagtgagcagctagcagaggagtgcgggtccctggagcctgcttgtgcaacgctgagctagtccaagggggaagaatggggtgcatggctctcagctgcagaccagcctggaacctctccagcctgctttagcagagacttgttaagaggtagcagcaggtggcaagattaggagccggagtagtaggctaaggctgcacttccagggacacactgcctctgccaccacccgtgccacgaaaatgggagcccaggaccctgaatctctagcagtccgtttctgaatcagttaccttgggtatgtgcctctggttgatggaaactaacttgtagccctgctgggtgagagcctcacatcgggacatgtgacagctttgttgaaagtagctttggaaacgcccaccacgtggggccactcactgtaatataaacggtcatgcatcactgagcaacagggatacgttctgagaaatgcgtcgttaggcgatttcatcactgtgggaatgttacagagtgtgcctacgcaaacctagatggcagagcccactccacacctaggccagatggcagagcctgttgtttctaggatgcacgcccgtacagtaggttactgtactgaatactgtaggcagttgtaacaatggtgagtatttgtgtattcaaacatagaaaaggtatagtaaaaacaatggtgttatggtccgcggctggctgaaacgttatgtggtgcatgactgtaggtataaagcattacagttgtttgatttttctctttttctcacccacagtcttaaggcacctcttatgccttttgtctgggatgtcccgggcagggttggaacgtgtggttaaggcatggcggaaactgctttggggacagacgatggcctcagcttgccttggggtgtcagtgggaaagataggagctgcccctttgccttcgtgtttcttcgtaataatctcagatgtacccgtctggtgggcctctcctagaaaaagccccggtgctctttgctcctgcggtgtttctcaggagggttgttgcttctttgtaatggtggggactcagggaagggacgcaggcagagggtgatgccacatcaaaaagggacccttggctgggtgtggtggcttacgcctgtaatcctagcactttgggaggccgaggcaggtggatcacctgaggtcaggagttcgagaccagcctggccaacgtggtgaaacccggtccctagtaaaaatacaaaaatacaaaggtggtgggtgcctgtaatcccagttactcagtaggctgaggcagaagaatcgcttgaaccggagaggtggaggttgtgatgagccaagattgcgccattgcactccagcctgggtgacagagtgcgactccatctaaaaataaactgaaaaaaaacaaaaaacaaacttgggccatcagcttcttggaaaggctggtgtgaggttgaagcatttgctggtgcctctgctcaacgtttttgtggtgaacctgagcaaagaggttatcattagtggattttactgccttacctgggtgggcactcccttgggaggtggatggacatttgcagctgagcccaggtgggggaattgcgctcactccgccttcagaattccaaaggctgggcatgcatcttggcttcctctaacccatgtctttctctaggtggccacagcagagtgtcattaagtatctattctttgcttttgttctcagggcaggaagatcccaacagtttgcgccataaatataactttatcgcggacgtggtggagaagatcgcccctgccgtggttcatatcgaattgtttcgcaagtaaagagagccttcctttttcctataacctccgaagctttcaccgccactagcaaaacatgagagctatttttgagatacattaaagtgtcaaagtgtcactgaatatcttcctacttaagataagtgtgtctcccttagaacattttccctattcgactatataaatctacattcttgacccttctgaatgtttaaagaacctcgggctctgaagagattctctaagaatattttgtaagtggaagtttttgatgcatgcaaaaaattggcaggatgtttagtgtttaaatgctaagcccgatatataaaggagcgatggctaggtgtgtgtggctgttgcacaacccattaatcaatgcgttgaagcgttcattttaaggtgctacaggcttaagtgtgtactcctttggattttaggcttccgttttctaaacgagaggtgccggtggctagtgggtctgggtttattgtgtcggaagatggactgatcgtgacaaatgcccacgtggtgaccaacaagcaccgggtcaaagttgagctgaagaacggtgccacttacgaagccaaaatcaaggatgtggatgagaaagcagacatcgcactcatcaaaattgaccaccaggtaagggtgttctcgcctgcagaggtgagttctcagatgccccggaacacccttggcaaaggcaccagagctctctgattgcagctgattctcggggggcactgaagccagtctgagccagtcacaggagggccttgaggagatgctgagtatggcctgggggtgtgggagaggaaggggctcaggaaaacttctgtaaggagccagataaaagtttttaaaataatgttttaaatgtttgtcaaagaaagcaatagatttgtaaagaaattagtaggtaagtagtgaaaattgattctccttcccattcccaatcctgtggcaactcttgttacagattttatttatcctccacagatacatcatgcgttcacaatgaacatagaatttactgggttttagactgagccatccttaacttgtcaacagttactttgaaaacaaaccagctctcccaaattggggttttgcggggttatgagatgtgtttcaaaagaatgtttcgtactttaaacatcttggaaaacttgaattaaaacagagctaatggatttcttctttccagaccttctcagagcttttagtatgctagtgtgcacgtggcttgcctacaaaagggtgttgactgaactatttgcccaaattataatcatttgagtatacagctttttgtgggggcaggcagaactgagacataccaaaatcagtttgggaaatgctgtatttgaaaatgctttctatttaaatattctctttgcaatcatttttgctctgttgatttgcttagcaaagtcttcatgtctgggacaatatccatttcttactgactcatcaaaaacccccactcgacacgtcgatgagagaggttttgtttgctgtgtggcatgttcagtgaaagcgtggtttccagtttcttcacatccttataattttctagacttcagatggagggaacaatcagaggaggctggaatcctgcctctgaccaaggaaaagaccagaggctgagccaggtggggtctcttgtccagccctctgcttgcctcgctttacctgggtgtgggctgagtaattccagacaagcgtggaattaatctggctgtttgtgctgttcagtggcacgctggttacacctccttctggaaacaactctgcgtgtgctgtttgggtggtaggattccgggtctccttctccgtctttttataacatcaagttgctgcccagctcaggctcctttacggccagtcttcagaaaaccaccagctaacacatttactaccctccttccccgatgttcctgtagcttctctatggctgggtggccaggcatggccgaagaggctctgggtagatataggctctgtgcccggtgtgtgtaactggccttgagtgaggctgcagttgtgtgttatttctattaggtcactgtggaatttctagcgacaactaatctttcaaagtgtgtttattggtcacaggattattgggccagcctctgccttcattctttttcacctaatctgcataatagctgtgttatccccattttagagaagaagaaacaggggctcagagaagtctagtaacctgtgtgaggccacacagcaaacacctcatgaccctgccctcctaaggcagcccatggctactgctggagggatagaggccggccccgtggtttgatgggacagcttgaccttaaacagcccatgggaaggcgggtgcatctggtttaggaacaggctgctagaaaggtatccaggatgtggtagtctcaccggaaggagccagtcagaatagcacagcctgtggccacgcgtgggacctgttcagcctcatggagctttgggaggcagccagcagcagggcatgggctgtgtgcaggcgaggcgctggcctggacgccgcccccactgcgtaacttcgtgtttggaatgcgtgggcacataccgtgcggctgcttctggccgggggatattcttttccaattttgagccaaggtggagactgtctcctcgtgccatccctggcatgtcctggcaagacgtgaacgatctcaatagacgagctttgcagagtgtgtctgacctgactcctgctgtcttgggagtttagctcttcagccagcagcatgctgtttgacatgtgtttcaagccccccaagaaagggtgcttgaaatttaaaattgaactgatgtggcttttcaaaatggaattggaaatgaaaggatattaaattgcagacacccacacaaaagactggtttccactgactaaactgcttttttttgctgatagtagttgaaagtagggagagtaacagcatctcttccagctttttctcttttgttcccttgttttgatgatgggttatttcgggggaagctctggctggccttgctttgtgtcatcttagggataacaaagaggatgaaagagatcaggaaaaccgagaaggcagaacagaaccagcagaaactgtgcttgaggaatgaaaatcacctacacggctccttgtcatatgagactgtggcccagcctcctgcaaagccatttaagagtaacccagtgaagctggtgagactgcctgccgcgtccgtgggcccagtgactaactcggtggcttatcatctgggcccagctcctcccctggcatcctgatttcacttggaggggcccccgttgtccttcataaacatgtttatttcattttatttttatgttttgagacagagttttactgttgcccaggctggagtgcagtggcgccatctccgctcactgcaacctccacctccaggactcaagtgattctcctgcctcagcctcctgagtggctgggactacaggcgtgcaccaccatgcctggctactttttgtatttttagtagagaccgggttttgccatgttggccaggctggtctcaaactcctgacctcaggtgatccacctgcctcagcctcccaaagtgctgggattacaggtgtgagccattgcgcgtggctgtaaacgtgatattcttgagactttcagtgaaataagaattgccacggacatctgtggtcattgtccacttgccactcacctacccccttttctggcagcaacagccggcatttcacatgtccatcatcggacagcgtaggtgggaccatcagtcatggtgtcctaccctctgtggccaaggagtggacacaggacccagttagggcaagcagaggctccccttggaatcgcaaagtgaagctggatgccacccacagagactaacatggtgaagctgctgtagcccctgctgttgagcccccagcactgcctgagttcttgcactttgtgagtccagtttaatatctgcttttcctcccattcttggagctcccctcacatctccagtggcttgaagttgccagagatgtttctgggcttgtgaccaaatgactccttttctgcttctcactgctgagcagacacatgtgcgctcactttgcctgctgagtcttgggacccggaagagcttttgggagacaatcacggaccagccccctcttgcctgccctgctgtctccctccaagcaggaggtgagaaggtgtccacctgcagccccggccaggcatccctttctgtgcttctgcccaaatctgaaattcccctctccttgggacccacgactggggccagcctgcctggggagggaatcccagctgcagaaagtcgggacagtgtgcgtgtaaacatgttaatagaaagcagctttgagggcagactagttcagcttcagttacaaactctttccaaatgcgtttaacatgagccactggctgtgcgcagcatatgtcaagctttcatccaatggtggcattttgtccctgcggggtttttttttcctgagcagtttggggcaggggtggggacagggagagagaaaagtaaaaagagagcagtttggtttcttcaggctggagtacaaggcagaggtaatgggatgtattgaagaaggtaggagggaaagttactttagctacagctatttgtccagctgtgctgattaagaaacttggagaaaagcatctttggaatcatgtccttcccatcttatatacagcctttgcagatttcctgctgttctgagagagatctgaactccttaccaggaccttgagggccccacctgattgggcacccctcactctctctgcccctcctccccttcccctcctcccctcctttctccacccccacctgctctgctcagacaccccttccttggttgcttcccacaggccagggctgtcccctggggccttggctgttcccctcccaggagcgcccctctccagctcctcatgcagccaaccttcctgtccttcaggcctctgattaaattctgccttagacatctctccccaccccgctgtgtgaggtagcgccccatgccccagtcccctcaactccactgcctcactttggggacacatcaccccagggacaactgcattccactcttggtttttccctcctcgtctatttatcacaatttagagtcgcctcactcatttgtcaaatgaagttcatctctgcagctggactgcggggttgggggcacatccggctgtcggtcctcaggtaggaggtgcttggcaaccttgttcagagtaggacgttcacagctgtctgccccggaggaagcaagggcacccgccacatggatggaattgaggggaaggcacccggggctcctgcatcgagcttccctcctatattcaatgaggaaatgaccctgcagaaggctggctgcagatgcccctgcctcccggctttgcctgcttggagtttgatggacacgtggtcctgtcagggctacagcaggtctatggtctttggtaacggaaagcgctggtgaaacagtgagctttcccgtgggtgcttttccctgacgccaacaaccaggtaaatatttggaaacggccttgttgaggcttgtgaggtggttttcctccctcccctgtaggcctgcgccacccccccaaccccacggccacctttgggccagatggcacccacagacctgtttgaagtggccacagagggagccctctgggcgctggggccgctgtgtttgcagagggtcctcttactgctgagctggctggtgcagtgagaaggaaggccgacacccctgatcctcatcaagttcagacgggggtcactgcgggtgaggggcctggggccttttacatgtcccgggagctgctgagcaggccactcttctccaggccaccagaacttggccctgcgcatggtgaatcttccctgagtcagctgagtgagggggttcaggcagccccccgggacatggcagtggcggggagtggactggggtggtgcttgccatgactcacgccggttctcctcaggcaaccggatggtcagatgcgctgactcagtggcctgagctcgtccaaaagcgaatcagagaacacagggcctgggctcacccgctgccctcttctggagtcatctgtcactcatcctcatgaaggaagcgcctgggagcctggaatgcacatcgcactgccccagctcccctcttgtttctgtgtttttccattttggattctttcccccaacgccttctgtactgggcattttgtggtctcttctttttctccgagaactctgagggctaccattgcatttgctaatgatgccacagacggtgttgacgttatgaggcttctattactgtattgatttttaccatttttagggggacgggaatcaatatttcatgagggaatgtgaagccagacagtgaagtagaagctggcttttattttgtgccaggctttgtccagaggcgggtggggacgtggctcctaagctcttgattgcagctccttctggcttgggaaacgtttcagttccccaaactctcagaactggatcccctgtgtgttctctggcccggattcaagaacttagttgattgtcaaggaaattctttggctatatttttctcttaatatggtaatgccttttttcactctggcactctcttttcagggaattggattaagactattatttatgggtctgacaaagcagttcccaagttgttgggactggatttgtttaggaatgtctcctgtcctcttcattgaggggggaatacaaattgcttccatttgacagtttatcaagtgtgtgacagagtatcagagtccagggttggccaactacagccagtagtccaaagctggccctctgttgttgtaaataaagttttattgggacatggtcatgctcacttatttaggtagagtgtatggctgcattcagtctacaccagcagagttaaatagttgtgatgaagaccacgtggcccgtgaagccaaaaatatttgcttcctggccctttacaggaaaaaaattcccagccccagtggcaggcaattaacaccttgtcctcgaggagctgaaagtggctggaggcaggaatgcttataagaaccaagcgaggtgaagcactaggtggccgcggcgagcaggaagagaagctgattttgtttgccctttcgtttgccagagattgtgggttctttttttttttttttttttttttttttttttgcagagatgaagctttgatcttgtcacaatagcagagggaggccttatttttgtctatttctctgtgacattggtagaaaggactttgtcagaattccaagctatttggcaattatccaattttgagatcctaatggatctttcgaggtctagtttgttcattcttttagtgattccttgttaattccctgattttataaatgtgtgttgaacatctgtcttggccaaatacttcttaggtgctgaggatgcagcaatagtgggcaaagccatggggcttaagatctagtgtgggaaatgggtgatgtaaagtaaatatggcgataagtacagtgcacgaagcaaacaagtgaaggggtagaaggtatcaggctgcaaagacagcagatagtgtaggcagggaatcttatctgagggggtgacatctaagctgagatggaaaggacagtgagagccagccaaggaaacaagttgggtgacaagagttgcaggtggagttgcttaatttcccacttctgctcagcctgcagatcctggatcttggactaattgcaaactgtcatttcctcgtgagtttattagaaccctccagaacaagtttctggttagctagtttctctgtgtgttgtctcatttcttgttggttctggttctttggggttcctactcatactctggaaagctccagtgtcttaagtagtcagtctcccaagagtctgaaagcacaaagattcacaatgatacgatcacctctcaatcatagcagcattgatgcagttccgtagctggtttcctaaagccatccagatctctttctgtggcaagagagaaataagaccttctggtgaattgaggactaattatcctaataaacatgcgaattaacagttcctttggttaaacaaagcaccagaatctgataatgggaacatgtgactcatggtatttccttctttgctttatctaccaggcagctcacagaaaccactggccttccctgtgttcccattttatgtcataaatatatatttaattaacttattataaaaggccctttgttcattgaccatatcaaattattcttatatagaagaggttatacatgttttaaacattttaaaataaatctgaaaagaatgctacatcctgggcaacttccctgcatttggggctcaaagaagctctatgtggttatgggtaatgaggagccagagtgccttcagggcagttcagcagatgctgaaaggctgctgtgtgctgttcgctgggcccaccaaatagagtaggactgagcccctgtccaccatgacagccgggagatacaagctgttccctttgcctccctgagccctgagctttatagcctatagacagctgaaaagcaggctgcatccgttacccagtcagttacccagacccaaatgccaggccttggctaaccccagttattacctaattttaatatcccaatggatgttttaagacctggctggttcattctttcatttatttacttattcattgattttgtaaatatttctggagcatctgccatggccacatgctgttgtagcagcatcagccactctgaagttggtggatgaaaggggatgcatcaaaggcgctgatgtatggaggagacgcaagttagacttgaccaagacaatattattcctcctctggatgccccgaatatatacagtcattagctgtcgggcccccatgtggcactgttgacattttgtggtttaaacactgaagagtaagggaatattggaaatggcaaacatctgatatagtgtaaaggagactaaatattttgatggtgttcataaacaccgaggaggaaagtcttttcatttttttcatttgtgtgctctctctttctctgtttttgcacactgtcctctgttctccttctccttctctttttccttttttctcccttcatctccccatttatctgatctctcccacctgaaccccttctaccctgctgccctcctgtccattctaccttctctactcccctccctagacagtagtaatcacatgtcagttggagaaacatgatggcaacttggtcacaccgttcttctcagtctgtatatgtcggtgatctcagtgcccatctggcagatccttcctgccctggctcttctgctcactgcgaccacccttgactttgtgatcactgataaccttcaccttctctaatctaaatcccaagcttctcactcttggtccaccacctcccagccttgtccgttctgaaccctgaacggaagctgaatggaaccctgaacggaagggttctgaagctgttcagaaccctgaatggaagctgaaatatcaatgggccattgcttttcacagtcctctgtgaaagattactggccaagccagcatctggagaattcctggtccaccacctccctgtctggagaagctggaacagccagctgcatgagcatgtgacccgtgtactcacaggccctgtgccctgagctcgctgttttaattttatctttgaatttgtatttttgtgaataaagccctatgagctaatggagcatgctcagggaacttggggctttagctcaggctggattcctcctgctgcctccccagtccctggtcccctgagaactccagccccatctgaccttcccttccctgtctctatgcaggggtcattgctaccctctatccctggaaaggatgtaggcacagggcagttctaggttccagcttgggcaccgcttaacatcttggtggtgcagggatcaggctgatgataccgtggttgttctgtgggctactgggcagggtcaagccactcccaccctgatccaggtacctaatgcacccgacacagaagcggcagtgtccttggggtcatccattatccatgtgttggaggagtgggaccctagggaagatgcttggctcgacttccccacccctagccagggcacaatcagaggtccaggggctggtgggcacaatgccaagtcgtgaggcctccagtgtctgcgctcactgtcccataaataaccacagtaataactagcaaatcaaaaacattgtgataggtcgagagagacagcatgtggaagaaaggaaaaagctttctattttagtacctttaacagtgctttctgtatgctttatgaacaaggagcctgcatttttattttgcactgggctctgctaattttgtagctggtcctgccccctagtagctcaagtcagcaaatctttggttcatctgagtccacagtccgctgacccgccctttttcacagttcctcccctgcccatgtgctcacttccctccttacccagcttggcgcactccctcaagcaagtctttggatgctgacatcccccgtaaacaacccttctgcggcctggtttgattttccttaggagacatgcaagttctatagcactgtttcttgctgggtatggaggatgtgctattttgtccattgcatattttttaaagaaaatgaaaggttagcataactgtttccagaaggcacattgaatcactcagttgagtcccagccagttgctgcaatgttagcctttgaagcaaacttgaaccaacacaggaccagcctagaagtcccagcctccagaaatgatgcagtggattctgcagattcagcaacaacaatatttttgtaactcaagagcacttagtaattttcaaaggagagaaagaagtaattgacttggcttattaggttgaaaaagagttgccaactttttctttggttttgatgttattggtttttttttatttttcttttctccaagcttcagggaatgagattgaatgagcactcaagtgctactaggcagaaccctgaatggaaggaagctgaaataccgatgggtcattgcttttcacagtcctctatgaaagattactggccaagccagcatctggagaattctaggaacgccccctcctcttgcagcagtataagtttgcggggatcatctgaccccattggggagttgtatgaaaaaggggatttattggggaccctgttgcctgtttggatcttacttacatttaactattgtctgctaatggattttttggaaagcaaccaggttttccgtaaagaatagctaattgtcagagctgagatgaccattggagatcactgggctcaactccctaattttagaggtgctaaaaccgcaatccagagaagctaatcaagtggttcaaggttgtagactgagttcatataggaccaagacccagcccagatgtcctactgtctgggacagtgttctctcagcatacgtggagcctgagggggtaatgtgtgtgcgtgtgtgtgcatgtatgcatatacacataggtgttttgcctaagttttcacttctgccccaccttggttgatcttggagaatgagcctgaggcgcgctgtcaacctgggggcctcattcagcacaggcccaacttttctgccctgggggagttccagcagttatggttcatctgtggttcagttatggaactcacaccacacatagtgcccccaaaaccgaggctgcgtgcacagacctcccctcccttcccgtggtgggcccctgcttgggttcttcctaaacttcccctttgccctgctctgtgttataccctctctggtcccctgtccctgtggagtgatccggggcacaagggcagctgtttccccgctgacctctgtgtgccctgagcatctgggaggtggggagcaggctggtgagaagaacacctggagtggaggttggggtcagggagggtcccagtcccggtaccacccccacctgctgtgggacctgcagtcccctcatcagcagaacggctatgaagccatcctgcccatccacagggtggtgggtcgtgaaggctgcatacctggcagagcgggagaagctctgggaagatgccggacacgcgccgtgggagtgatttccctgccttgcccagattctgctcccatcacctgaacctgcctgtcaccaccatggaactgctgtgaccattgctttccttttaagcagattagcagacatctcctgctccaccctgccaaacaaacaaacaaacaagcaaacaaacaaacaaaaatgtgcatgagggagtatggacttgtagagtcttttctaaacattgttaggtgcttgtattgggatcctctcttaaaatgaaccatattccccaggctttggatgacactcatggttgcccaccctccaacttccttccctgctggcagagccctgggtttgttttagttccaaccctgaccccaccgcattcctgactcaggcaaattcgcagggtccaatgcagtcaggggagccacgttccctcctccaacgagtgctgaggtcgctgcttgattggatactgccgatgacctacgaggaggagggtgccagggcgcttttgggactttgcttttctggagagatgcttccacagcatggtcatggacacagtcacgtcttgatgtgatgtctggaatggtggtggccgtcttgtggctgtgagaacaggctgaggttgattggatggagggaaggaaggagccttgttcttgatgctgtctgtgagcctttgagttatcagcctggtaccacccagcccttggacagatatctactctacatactccatttggagtttttttttttttttttttttttttttttttttgtcacttgcagttgaaaacaccctaattgatacacacaaactatttttagtgctggtctgtgtttggcccttatggaagactctgggctgagctgcccatggtgagggaggtggactttgtgttttcttactgctctgtgtcctggtggcttgtttgtgtctctgcccatgagacaaaagccgagagggcaagggcagattttcttaatcatatgttccctgcaccaagctcataggagacactcactgaatggttgttgagagagttctctttcacggaggcaatgttttgtgaaacgatgctgcttgttgttgtctgttggttgtaatatgcatgaacactaagagccatctttaatcatgctgtgggccgcctcttccaaggtgttagcattactcccactacctggtcagcatcctgcctatggctaggactttgcaatttacatagatatggtggggagacctggagcccatggccaggactctgacaccctcactggatctgtttctacatctacctggatggccgtctaggacattagaggatttgtgtcttcctaaagtccctctgttgagagacttctggctctgttaagaggacactatttagcattgtgagtccctgcaggctgggggccagtgggcgtttttcttctagatgccccctctcttcttctggcctcccaggcttcctgctcctgagattgtgagaactggcctgtgctgggctcactgcagaaagactgtcgtccccaaaggttttgcaccaaacttgagctacaagatcttttagggggacctgagatctccgcctgggctctatgagagcaggcatgggttgtttttgccccgtcactgcagtcatgcccacacttgcattttcttttccccccagcagtgtgaggatctggcatgaggagtgggactcgcgtgccctctttcttctcctcttccctctggccttttcatccgtcagtgggggacagatgtttgccctgtttacttctaggcttactgtggggctccagggagatggtgaagtggccaaggagaggagctgccaccttcaagacggcctgtggccggtgccgctttaaagggagactcagaggtgctttgctgtgggtggcgcgggaaccagcctggggacagcagtgcagaggccttggactcagagtgcgtgggccccgcggggcttcacggcgcctgtggctgtgcacttccagccatatctgtgctgcatctcttccacattcccccatggagctgatgtctagacagctatggaattaaatgctcaattaccgagtaggaatttggccagcagaggtatagctgctgagtagacagactcgaggtgaggctcacggctgagaacaggccccatctggctttggaatgagctgaggtgcccgatgctcctgcagccagtggctcctgtggggagctggggccgtgacccccaaaaggcagcttgacctcatggaccaccataaatctggcctggtcaacatctctgccagacatcattcccttgcaaagatttctgcctgtgattggaattctggatgaacatgtactgggcgtgtgggtctgacagctgggaagcttgttctcttgtttagccaggctgcccatcatctgtaagcctcagtatccacatctttaaaatggggggaaaatatagctcaactcctaatggtgccatgagaatactttgtcacctgccaggcaaaagcttattcctttcacagaaatccagggtttacaatgtgagacccctccccactccgccgcatgtgtctgcttgcttttttctgtcttagggttgcccttcatgagctaggaaatgtctgagtggatgaaaacctaaacgagatgatcactggtggtgcccattggtgcagcctttgcctaaatggctacttacgtagccacatttcctcgtctgtgttcaggtgaggactggttcctgggcagactgcctgggtttgcatcacgggtgtccatcttgtcgaagcccatgtggtcacccaagtgtgactgagccaggcttgcccacggggtgctctgggccccattttcggcagcaggcagcgtcccctggaggcctggccctccccgggagcatggggagtagcgcctatgggcaagcagcctgcagcctccatccctgcctgggggctcccccgccccagcctcacagcttctccaaaagtgtttgtctccttgccgcatcctctaggcctgagctcagacggtggaaaagaagagctggaaggagagttgcctttcagtctctctgccttctgaggtctcctgagacatagagcctgggcctgcctccctttctaggaggcgccaaggggtggtaagaataggggatgagtgagatgtgaattaggatccccacagcaagccctgcctcgtaactttctgatgggttttcaatgtgtggtgaagcagacgcctgctgggcccccttcctgagttgagtttgacctcctgcctcctgtctatctccttgggcagccaggccaccccgctccattaacctgtgccaccccatccctttacctgtcgcaagcccagccctgaaggcctcaaaggcctggtcttccagccagtccagggcctgaagggatggcagtgtccctggtggacctcccctggtgtggcctagtgcacatcccagccctgcctcctgccccgcctgcacgccatgagtgctgaagtcatgcctggcaggggctgctggcccaggcccagagtaaacacactgcgctgagctcgctggtgtgctgctggatgctgatgagcttgaggagtgtgggaagtgagcatggggctgagtagagatgcggcaggcctgcacctccccgcagctgccctgcatgctccagcctcaggcagccacacagggaaagggtcacccactgtcagggcagacctttaccatggctgggtgacacgggctggctgtggaaaggtgtttggtggttcccgctgttggatttgcacaggcccagatgctcacagcaaaaccaacacctagatggtgcttacaggagccagcgggtattcaaagagctgttcagatcttaagttgcttcattctcacagtggaccattgaggtagctgtacgttagtcccattttccagatgagaaaactgaggacctgagtggtcataagctcaggccctcatctaaatcacgcagcctggccccaggtgtgtgctcttgaccatggacagtgctctcctggtcctcttggtatctgtgatctgagggaccttcctcctcctcagtctcgtatagtcagttttaggtcttggactctgtcttcatatccctttctcccttcgtgagctttctcacccagcaccttccttatttggtgtgtgttgggggatatttgtggtgtggcgtggcactgtgtagtggatgagagagtctgtttttccgatcccagtcccaggtttcaaaccctgctctgtctcgagtcacccagaatcttggaccctcagtttcctcatctgttaaatgggcatggtggtcaccccacctcatcagctagtgtctgctccatccctggtggaggagatgactcaagtaacccctgggttccacctgccccaccccactggtcccctggctctttctttgttgagatagacgaatgtgaggctctggagttgcagttcccacgagggctggggtggctgtctgatttctgggcctggtccatgttgttcagggcagctgctcgttctaagtgaataaaggctgaaggaactcgggaggtctgctcggctccgaggaaggcagagagggaaagggccccgatgccttccctgatagagctagggaggcccttctgtggttccccccagctccttggcctgggtgaccctggagctggcttctgttccattttgttgtgcagagttgtttgagactcctggctttgcctggcctttgtgggacgctggagatcagggcttctggagttggccaattagcctgcccagaccaggaagcacaggtggctgacagagggccgtttcaggagaggagagacagcctacctattcggtcttgctgtccccatgctccatccctgcccctgaccagtgtggccctgtactcagcataggcgtgcacctgagtcagtacagttccctgcccgcagagcaccccaaatattccaggcctcaggacggatgtgcacatgatgagtcggggcaggtttcactgcctgtagcttgggatccttccctggggcttggttctctagggccatccccagcagtctcaccccaaaccctaaattcatgttgtcttcctctgtctcttggcctcaaggtttcagagtgagtctgtgctgatagcttcaagatgtgatgagaccccgacttggcctccagttacctccccacggtttccttggtgtgtgtgtggcttcagtgttcactggctcccgcacggcttgcaatgtgtggattacgggtgggagggaaatccagtcctgcccgcagcaaagggatgttagttgtgagctcagttccccaccgggcctggtgtttccaaatagcccgtcactgtccctgcttggttttccatgatatctgtgcctttacctatttggttaaattaaaccaactcagcaacgccagccattgtggtttcagggcaagctgcctgtcctgctgcttggccgctcctcagagctgcggccgggagagttcgtggtcgccatcggaagcccgttttcccttcaaaacacagtcaccaccgggatcgtgagcaccacccagcgaggcggcaaagagctggggctccgcaactcagacatggactacatccagaccgacgccatcatcaacgtgagcctctgtccctctgcgggtggggattggggcagagttttgccagggggagaggagtcagcataggtcttagcccctgactttgttgtagtctgcgtgaagggatggaactagaccaagccatgtggattctagtgccagcagcatggcaggggtcacatggcggggacggtgacaccggagcaggtggacagccagcctcctcccaggaggaagaagttgtattgggtgctttagggtgattgcagttggcttctgggcttcagagagaaaatctccctgtttacggcacctctaaaactttctgaaaattgttaaggtcatttttttccggcaaaatattaggttaatgggaatgaatctcagagaagaatcgtgccccccactctaggcaccgtgctcaggaaacgaccaggcagggacatagattgaaccatgttatgacacgatttgtaaccttttcatttctgtttaattgcagtatggaaactcgggaggcccgttagtaaacctggtaaggtcttttaaacctatgttaggtcatttgtttttatctatgtatacgctgttttttgtttgtttgtttgttgtttgtttgtttttgaggcagggggtcttttcaaacataaggttgccaaagtgtattataaattcctttaaaatggctctgtaaatgtactgcgtgcttgcaaatgaccctacggatcttttctggaaagagtaaggcaggccggaggtgagggttggaaatgttatgccagagaacacacttgtgtctcagagttacaggtaaacaccgtgaaattcagggccaatgcaggagtaaggtgaaggtcactaaaaatgctggccagtcaccgaaagcacctcctccaaattaaatctcctgggctgctgaaggagctggctgggctcatacacattttctcttggccaggaatcctcccttaaggcctggctggaatgaggaggagttacccacccacaaagatatcacttaagtcttcccttaaatacttgagcagaaaaagtgaagccttagaacacagaccagcagagctagagggcagctctggggccatttatagagggcagctctggggccatttatagagggcagctctggggccatttataggggctgtctttagcaaggcccagtgtgatggcacctcctagatggtgccttggcatcaggtactgacatctcagcactcctgggaagtgtgcacttggcagctttctcttcccagcagaggggcagctgtgctcccagctctgtcctctgcctccccgcgcagcacttggggatggagtggagatggctttgctggtaatgaagcatgacagccctaagctctagggttgtttccccctgaagtcagcagagtcatcttaagatcattagacatgggagaagcaggaaggtgtgggcagccacctaaaggagtttgagcctttggaaacgtattccttgtgaaacaggagcaaatcatatcgtgcattttgaaactatctgtgcttaccgtgaggtgagcacccagtgccgacctggagtatgtgcgattcttccacagctgcgcgtggctcgcgctgcctgggtgtcctgatgcctctctccctgctgccacggggatcccctccttgcatctccccacttcgatctctgaaatagctcagggacttctttcaggcatattctctctgggtgtgtacctgccggtaaagcttcacgattcagtaagccgtgtccttcttgcttttcaggacggtgaagtgattggaattaacactttgaaagtgacagctggaatctcctttgcaatcccatctgataagattaaaaagttcctcacggagtcccatgaccgacaggccaaaggtaggcaaggcccacacagccctggggactccggagatggggcctgaagctcagctgccctttgggacttggggaagggaaaagcggcagcccctaggactagccaagccgtctctgatccagaagtgaacgggaatgcacattactaaatccctcgcagaaggtcacagacatttcaccatttttgtcctctgatcatggcaatgtcacttgagtcagtctaatatgtaccaggcatgatcctaggtgacttgtgtacattatttcactttctttatgtatgtcacttaattcttttgccctatcagttaggaattactagtcccattttgctgatgagaaaacggttcagggagatcattctgcaaacgtttattgccccatctgctctaagtcaagcagggagcttggcagtggacagctcaactggggcctggggctcaacaggggcctttgccggtgtgacttttatgttctgttgggggatgggaaggctgacagtaaataatcaaacacataagatactattagtgctcccaagaaaacggatcagggtggccgtcaagggagcgactggaggggcagctggtggagatggtgtggccaggaaatgccttccaagctgaggtctgagtgaggaggaaccagcgggcagggatgtggggggaacactccagaaggaaagacagaggactcagcatagttgagtgagcacaaggcccctgaagtggcctgagggccggagcacagtgacagcatggagttccccggggtggaaagaggccaaggccgggcgagcaggctcacagcaggccgtggtgagggacctgggttgcatcctaacgacatttaagaacagggaagtttatgatctgattgatgtcactgaaaggacactctgatggctgcggggagtctgctggaggggttgctggaagttggggaccggttaaggggctctcccagccatctggatgagacatgctggggtctcagacaagggtggtggcagtggaggtgggacagaggggtcacattccagatatatatggggggtagagcaagcttggggaagggccagctgtcaggatgaggccatgaggaattaagggtcatgcccaggtacctgaccattaattgaaacaatgggactttcccaaggtcccccagaggggaggggtccagaccaggatttgagccgcaacctcagtgtacccttctgtggcccttcctgcaacctgggggattgggcccccggcccctggtgtccccagcacccccaccaactgggctgaccttctgctgtccctttgttgtctcaccaggaaaagccatcaccaagaagaagtatattggtatccgaatgatgtcactcacgtccaggtgggtaaacaggatgcgtgtctgtgtcttaaattttaataaacctgaacttcagaaggtgctcacgggcacccctgaaagagaaaccttatgctgccttaagacgtctcagtttctgcttataatgaagtagcatcgggaaagaggacaggtcattagccttggcccctttgtttggttttaacctgtgtttttgcattctgagctggttttcttcactggcagcaggccctccggtgtagaaggttctgccctcctctttgaaggcaggcctgaacagtgtgtgcgtggtggggctgttgattcactctggctcacgtcttccttaccccacattctgttgaaacccacattccaggagggccccaagcccctcccgcagctctaggcactctgctttcgttgctctgcagctcgtgggccgcggctccaggaatgccagggcaggtccagcgcagggaagtgaatgactgatgtgcttgttttccccgagctggtggaattgcggcctgtggttggcaggctcatggcatcctggtgttctaaactggatgaaaaattctggtgtaatctcatgagtcctggtagtagactcacctggcatggctaaaactgtcagaggtaaagtaggtaaagactagaatatagtaacagatagattaatgtgttcattactatgatgaattaatgattcactcactgtgaaagtattaatatattttgatacatgttatgaatggtggtccctttcttagcactccagaagatggagccatttgtcaaggttaaagtgtcccctcagttgtttgcctttggaactacgaggtgtagggaaagatggtaagcccttggtgcccagcttcctgggttcctgtccctgctctgatatgtcctgccttgtgaccttgggaacgatatgacccctgagtgcctcagtttcctcctcttcaggatagggatgacagcgcaggtgcttctgatgtgtggccaggctcagatcagggagtggtggcaggggtcaccagccacagtgatgccagccactatgtatcacacgtactgggccaggtgccttactgggatgatctcatctgatcctcacaactcatgttgtagggtactgttattatccccattttgcaggtgaggaaatgaaggcacagagaagttaagcaactgtccgaggtcacacagctagcaaatggccgagctagggctgcaaaccaggccaaccactgtactttactgactccttagtaatagctactattaattaagaaataataacaatgatgatggctgggtgcggtggctcacatctgtaatcccagcactttgggaggccaaggcgggcagatcacttgaggccaggagttcgagaccagcctggccaatttgtgaaaccctgtttctactaaaaatataaaaaattagccgggcttggtggcaggcacctgtaatcccagctactcgggtggctgaggcaggagaattgcttgaacccgggatatgtaggttgcagtgaactgagatcgtaccactgcactccagcctgggcgacagagcaagactctgtctcaaaaaaaaaaaaataaataaaaaaaataaataaataataaagcactttccttgctgttaccaagtaaatctttgactctggtagacaggcaattttaattttaaaataggatcagaattcctggaggaattttaccttagacctaaggagaagacgggaactggtgagagctgagttttgcgtgaggaaggcctggtgtttcttcacactaacacgggtgctttttctctggagcagcaaagccaaagagctgaaggaccggcaccgggacttcccagacgtgatctcaggagcgtatataattgaagtaattcctgataccccagcagaagcgtgagttggagtcgttttctcttttcccaatattcttgttgttcctgtgggggtagcaggaagagggagcgctgttccttttctactggctcagatgattatgttgatccttgacagacgtggtcggacgttgcttgtcattcctgctggccaggccttccgacctggctcggctcgggactcatccataggagggtgccttctgtcttcaaaagtccttgctccacgaggaccctccagatggacagagcaatagcagactcgtaatgagtctctgagatggcccggctggccagagagagggtttcaggaacagtgtccccaagccctcacttggtggtccttttctaggcttcaggacccttctcttcctggagtcttccagaatgtctctgacaattaggcccatacctgtcaacacctccagaaaaataacccaagtgatatcaaagtaacatgacaagaagtagctcaaccatccatcagggtttgttacctgtattggcggaatatccagagaaaagtgcgagaccagggaccagcaaatgtgccttgggggctggatctggcccactgcctgcttttatatggagctgtgggctaagaatagtttttgcattttatttttatttttacttattttttattttcataggtttttgggggaacaggtggtatttggttacatgagtaagttctttggtggtgatttgtgaggttttggtgcacccatcacccaagcagtgtacactgaacccaatttgtagtcttttatccctcatccctgtcccagcctttccccttgagtccccagagtccattgtatcattcttatgcctttgtgtcctcgtagcttagttcccacttatgagaacatttaaatggttgaaaaaatcctgaaataagaatagtattttgtgacatgttaaatttgtatgaaattcaaatttcagtgtccactgtaatttggtttatgacatctatggtggcttttgtgctggaacagcagagttgagtagcttcaacagagaccatatgtactgcaaagcctaaaatatttcctatggagccctttacagaaaaagtttgcagacccttgtgctagcccatgaaggaccatgacagcgttttgacgctgagctatataagagctacagttatagtggcaaccacacaaaggaagtgcctcttaacagaaacattccgcccacccctataggaactgcattctgagttgcaatacccattataagcaagttggccagatagtggccaactatctggcagatatctggccaactacgtggcagatagtacctggtacatccttccccactttggggtcaatcttgacctttgatctccttggggtcataaagccacacaagtgttagtaggcatttctacagtggacacaatggatgatttagcctaaaaatctcaaaaggagcccagcatcctggcacatgcatgtaatcccagctactcaggaggctgaagcagaaggatcccttgagcccaggagttcgagactagcttgggcaacaattgagaccccatctcaaaaaaaaaaaaaaaaaaaaaaaaagagtggggaaaaaagaacattattaaaaaaaaaaaccttaaaaagtaatccaatctaccgatggtttattttttattttattttattttttttgagatggaatcccactctgtcacccaggctggagtgcagtggcacaatcttggctcactgcaacctccacctcctgggttcaagtgaatctcttgcctcagcctctgagtagctgggattacaggtgcccaccaccaaacctggctctttttttttttttttttgtaattttagtagagacggggcttcaccatgttggccaggctggtcttgaactcctgacctcaggtgatccacctgcctcagcctcccaaagtgctgggattacaggcatgagccaccgtgcctgacccactgatggtttgaattattctaagttcgccaccgtccaatcctgtttgctctgggcttttaggttctaagctgtgcctctgtccatgtaaagtcagaccaggaggaatggaaacacgaaacattgccattgtgtttccctttgtgttgcagtggtggtctcaaggaaaacgacgtcataatcagcatcaatggacagtccgtggtctccgccaatgatgtcagcgacgtcattaaaagggaaagcaccctgaacatggtggtccgcaggggtaatgaagatatcatgatcacagtgattcccgaagaaattgacccataggcagaggcatgagctggacttcatgtttccctcaaagactctcccgtggatgacggatgaggactctgggctgctggaataggacactcaagacttttgactgccattttgtttgttcagtggagactccctggccaacagaatccttcttgatagtttgcaggcaaaacaaatgtaatgttgcagatccgcaggcagaagctctgcccttctgtatcctatgtatgcagtgtgctttttcttgccagcttgggccattcttgcttagacagtcagcatttgtctcctcctttaactgagtcatcatcttagtccaactaatgcagtcgatacaatgcgtagatagaagaagccccacgggagccaggatgggactggtcgtgtttgtgcttttctccaagtcagcacccaaaggtcaatgcacagagaccccgggtgggtgagcgctggcttctcaaacggccgaagttgcctcttttaggaatctctttggaattgggagcacgatgactctgagtttgagctattaaagtacttcttacacattgc

SEQ ID NO: 5 - DNA, 2123 bp, Macaca fascicularis
atgggctgggccgcgcggccgcgcgcactcgcacccgctgcccccgaggccctcccgcactttccccggcgccgctctccggccctcgccctgtcagccgccacggccgccgccgccgccagagtcgccatgcagatcccgcgcgccgcgctgctcccactgctgctactgctgctgctggcggcgcccgcctcggcgcagctgtcccgggccggccgctcggcgcctttggccaccgggtgccccgagcgctgcgagccggcgcgctgcccgccgcagccggagcactgcgagggcggccgggcccgggacgcgtgcggctgctgcgaggtgtgcggcgcgccggagggcgccgcgtgcggcctgcaggagggcccgtgcggcgaggggctgcagtgcgtggtgcccttcggggtgccagcctcggccacggtgcggcgacgcgcgcaggctggcctctgtgtgtgcgccagcaacgaaccggtgtgcggcagcgacgccaacacctacgccaacctgtgccagctgcgcgccgccagccgccgctccgagaggctgcaccggccgccggtcatcgtcttgcagcgcggcgcctgtggccaagggcaggaagatcccaatagtttgcgccataaatataactttattgcggacgtggtggagaagatcgcccctgccgtggttcatattgaattgtttcgcaagcttccgttttctaaacgagaggtgccggtggctagtgggtctgggtttattgtgtcggaagatggactgatcgtgacaaatgcccacgtggtgaccaacaagcaccgggtcaaagttgagctgaagaatggtgccacctatgaagccaaaatcaaggatgtggatgagaaagcagacattgcactgatcaaaattgaccaccagggtaagttgcctgtcctgctgcttggccgctcctcagagctgcggccgggagagttcgtggtcgccatcggaagcccgttttcccttcaaaacacagtcaccaccgggatcgtgagcaccacccagcgaggcggcaaagagctggggctccggaactcagacatggactacatccagaccgacgccatcatcaactatggaaactcgggaggcccgttagtaaacctggacggtgaagtgattggaattaacactttgaaagtgacagctggaatctcctttgcaatcccatctgataagattaaaaagtttctcaccgagtcccatgaccgacaggccaaaggaaaagccatcaccaagaagaagtatattggtatccgaatgatgtcactcacgtccagcaaagccaaagagctgaaggaccggcaccgggacttcccagacgtgatctcaggagcgtatatcattgaagtaattcctgataccccagcagaagctggtggtctcaaggaaaacgacgtcataatcagtatcaatggacagtcggtggtctccgccaatgacgtcagcgatgtcattaaaagggaaagcaccctgaacatggtggtccgtaggggtaacgaagacatcatgatcacagtgattcccgaagaaattgacccataggcagaggcatgagctggacttcatgtttccctcaaagactctcccgtggatgacggatgaggactctgggctgctggaataggacactcaagacttttgaccgccattttgtttgttcagtggagactccctggccaacagaatccttcttgatagtttgcaggcaaaacaaatgtaatgctgcagatccgcaggcagaagctctgcccttctgtatcctatgtatgcagtgtgctttttcttgccagcttggtccattcttgcttagacagccagcatttgtctcctcctttaactgagtcatcatcttagaccaactaatgcagtcgatacaatgcgtagatagaagaagccccacgggagccgggatgggacggggcgcgtttgtgcttttctccaagtcagcacccaaaggtcaatgcacagagaccccgggtgggtgaacactggcttctgaaatggccagagttgactcttttaggaatctctttggaactgggagcacgatgactctgagtttgagctattaaagtacttcttacacattg

SEQ ID NO: 6 - DNA, 52575 bp, Macaca fascicularis
atgggctgggccgcgcggccgcgcgcactcgcacccgctgcccccgaggccctcccgcactttccccggcgccgctctccggccctcgccctgtcagccgccacggccgccgccgccgccagagtcgccatgcagatcccgcgcgccgcgctgctcccactgctgctactgctgctgctggcggcgcccgcctcggcgcagctgtcccgggccggccgctcggcgcctttggccaccgggtgccccgagcgctgcgagccggcgcgctgcccgccgcagccggagcactgcgagggcggccgggcccgggacgcgtgcggctgctgcgaggtgtgcggcgcgccggagggcgccgcgtgcggcctgcaggagggcccgtgcggcgaggggctgcagtgcgtggtgcccttcggggtgccagcctcggccacggtgcggcgacgcgcgcaggctggcctctgtgtgtgcgccagcaacgaaccggtgtgcggcagcgacgccaacacctacgccaacctgtgccagctgcgcgccgccagccgccgctccgagaggctgcaccggccgccggtcatcgtcttgcagcgcggcgcctgtggccaaggtactctgccgcgctcctgggcagcaccccattctctccatcccagctcggacctgcttctgcgggactggtgggcagaccgaggggcagcgaagcgttgcggggtggccagggcaactctcggggacaggcaggtgggccccggggtggcggctttccgcgggctgcctcggaaacgagcttcgcgcccagcccgggccggttctgcgcccagacgatgccggtgcgccgggcctgcactctggggctcgagacgcctggcgacctgccgcggagcgccctgagggcagccacacagcgcggggagccgaggacaaataagaggagtgggggcataaagggaggagagaagttcaggactaggaactggagccttgcagagcggcttcaggaccacaagaagtcatttctgttgctttttctatttgcttcctccgtcccctttaaaatgcattactttgatcacgggaccgctccgtgaaaactgtatgtaactcttttggaaaggaagagtgtttgccggcccccgccggagtttccccaaaaagtctaccccgggcagggaacggtttggcatcgcactcgtttcggcggcgttgctgcctgtgttgctttcctcgttttgagccagccctacaaaaatgaaagtggctccttttgaataagctgaatcgggctttggatcacgaaatctgcagaggcgtagaagggaccgggttagtaatgaggaaggagcctacccctccctcctgccgcacacaggacctgttcggcaggggagatggtggtgatgggggcaggagtggagtggagcaatgtctaactctctcgcgggaccttccggagagatgcttcccatcttcaggcagaggccatgtggaagaataatatcgagttcagcggcggccagtcccgcggtgtagaaccagccagcggggcttggcagtgcgcttaggcgcagccatgcggctgctgcccgaccccagcgctgcctcctcaactcgggcagtgccaggagaggggcataggagagcacagtgcagagggactggtctagattttactttataggaatatggttcagtatgaccaactaggacttggcatagtttggcttacatggaccggaaggtgccagagccgaattgggtgaaattcgagattgtgtatttcactaacgcaggagcacagccctcgggaaactcagcctagttaggcagtagagagttgtcccggagacaagtgatcccgcagactagagaatgggcatgatgatagcacacgcctattgagcactcagtctgtgtgccgggtgtgttacctctgtgacctcatttggtctcacgaggagggagtttctcctctctctctctctctttttcttcttaagagacagggtctccctctgtcgcccaggctggagtatagtggtgtgatcatggctcactgcagcctcccacccctggactcaatgattctcctgcttcagcctcccaagtggctggggctacaggcggatgccaccacacccagcttctcattcctgttttacagatagcggaacttaggttgaaaaacttgcccaaggtcactcagctggagtttaaacccagatagcctcattcagaggagtcaggccagcacttaactccaagggtgtgggagaggggtcaggtgctgtaaatttccgggtgggttggacgtgcatccccctcagagccgggaacagcatacacaaagcctaagacttgtttggaggtgaatagatcagtgtggctgggggatgtttggggagggcagcaggagtgagccaggctgctggcccagagtcccagggctgaagaggctggctgtgccccgggccctgtgtgcagatgttcttgaactggggcaactcaaagcctagtgtagtgtagggctgacctagcagtggtgtgcggaatgcatccagggtggagagtttagactactgcaataatctgggtgtgaggcaacaacattgaaaaagcatgtttttgtccaaaacaagccagctgttactggtctcgctgtttgtggtctcattgcacggggtcctgagttgctggcaccatgcgagtcgcctaatttattgctagtgaggcaagttgcttaataagctttggagttggctgagtccctgtgtggaggaaaacaggtcccccattggccatcaggctcacggcgggccccggtgtaccagtgaggggacagccacagagggataagcatggtggctttgaaaggagggagagacagagtgggtacaatgctgtttttatccctccctccttcttttgcaaatatttgttgagctccgtagggtgtctgacaccgtttgcatgtttgtctggcacaccagaggcacttggtacgagtggattagtgaatgaataaatgaatgaatgaagacaaatgggaggtgctttcgatacacagccattctgtttttccttagtggaaggcactgctttgctgcgccccctctctggatctcactctccacccttgactttccggaggtgtttccgaggacaggcgcctgggagccagcagacttcattcagtccaagccaggctccaggactcagcagctggtgcctacgggcaggtcacttgacgtcactgttaaatgaggtgaattggctgcctgctctggctcgaagattggcgggagagctactttagctgcaatggacatgagccttttcatggggtgccacttgactagaggcctgaagttggagcaaggcacacacagatctgaagacagagctctcgaggcaggagcgggtgctgtgatttcaaatattacaaggaggctttgtctggggcagagcatgcgaggggatgagaggtagaaatgtcatcagatcaggggtctccaggcaggtgaccagtactttgggtcatggtagatctttggatagaggaacgtgtcaccattcaaaggaaggtactttcatttgtaagctgtttaatgaatagacctcagagaacatctctgctcaccgctctggaaatgaaggcaaatcatctatttcagaagtcaatgcactggcagggtttggatggcaaagtatacaattcaactagagaacaaagatctgtcatctccagctctgctggtcagatgattacaaaaaagaaagggattgaaatactaataggatacaaataatgagggctaacatatatgttgtgcttattctgtgctgggtgcatactaactcatttgatcctcctgacagtcctgtgagtgagtgctgtagtcttccctgggttacagctgggcaactaagtcacagagcagtaccttgctcaggactgctggtcccacacaactggatccagagtctcgttcataaccagcatgccgtgccgttgacagagcaacagagattataaaccacccccagctaagccccagctaatagctgaaatcaacagagctccagatggctgtggccttgagatgaaacaggacagatcacagccctcactcagcaggctcaggttgacagggttgcctccagttgccatcagtgcagccctcactaaagaaaagcaaaaagaaccgagggactgtaggaaagctgtttccacgccagagatccagacagcaaactgctcttgaagagagaaagcccttccggattcccccatgtcccaaaagaccagccacgattccagacctctgctaaaacacggacaagaagccaggatcaaaacctgaaacagacttcccaaacagcagaaccctcatccatttctcctcctagtacatcctccaggaaaggccacccgactcctgacaggagcccagacaagcttggaggtctgcaagctgcaggggtgcccagaaactccgcctctggtggtttttagtattgcctgctcctggtctcaccccagagcctctgaaggcagaggctgtacgtacatacctggtgaagaaccaagggcttagacggttgctttacttcttggaggcctggatggtttgtaaaatttatttatttattaattttttttttttgaaacagagtcttgctctgtcgcccaggctggagtgcagtggcgcgatctcggctcactgcaagctctgagacctcgcgagttcacgccattctcctgcctcagcctcccaagtagctgggactgcaggcacccgccaccatgcccggctaatttttttttgtattttttagtagatacgaggtttcaccgtgttagccaggatggtcttgatctcctgacctcgtgatccgcctgccttggcctcccaaagtgctgggattacaggtgtgagccactgcgccggcccaaaatgtactttatttaggtgattctttcatgggagcctcaaacaagcaatcattgttagctgagtgctgaccctgtgctgagctctggggagacagggttgaataaaacaaagtcactgcccacagggaacttacattcaatacattcagtgcaatcactgcttccccaggttgcatttttccattgttagagtgggcggtttgctagagagtcatttccactgttggcaattcaaatacaccttttgtcacttaaaaaacaggtgtgccgggacctgagcttcatcttagggtaggatgggtggaaacagttgtgagtctccagtttttagtcacccgaaacttggaaacttggaattcttttgagcagtttatgaggctctgcctgctctggtcagctgccttcttttattgctctgttggttttgctaaagagttaaaatattaaggtttcgtgaaattaggacgttaacaagctcaaaaaccaagtgtctgagttacttcattccactgagagagctgtaaatgggttgcattggaacttaaaataactgcattgagtaagcgatggtggcgggcaccatgagctaactgtggtcagaagcctgacagcctctgctttggggctggattctccgtttggagctgtgtgatcctggacgagtttcatgccttggatttagaaatcagactttccatgagcttatatttcaagtgaataaatagctctggtcaggcttagtttgaagaagaagtgagcttggcagtgggtgagggttcctcggaaggccagctggggtggaggggctgaggacaagcggctctggcccttcccgggttgttacctgatcaggtaacggctccctcgacctcttgcagcctcggcagtaaggggattgggccagttgatctctgaggctccttttaactggaatggtctgtgattcttgtaagaaaacaagtctctgaggaggttgtggtcgcctcattcctaatttaaaggttgggaaggcttccttaagagctacttctttttcctaaattattgacggttaaagccaaggctggcatcgaatggatgtgatccatcttgagcctggttgctttgtgtttcagctttgtactggctgctgaaagtccccaggagaccacaggggtgacatgttcatccccaagagatgagcttccaagagcctcatacctcttgctccttccctggagcctccaggcctttgggtagtcggaagtgagatacctttgtgtcatttcatcttttccatctccaccttctctgccattgaaaaaaaaaaaaaaggaaagaaaaatcctattaatagagaaaccgagaagtgtagccattctgaatgtgtttccaaaaggctcctggaagtggcatggaagttggagtgattcagcactacttggtgacgtgtgcctagaaccatagggggacattagccaggacaacacgcctcaggacagaagtaagtggctgtgaagaggcatgtccgtcactgctggaaaggcgcagagttcagcttttggagtcaatgctgagagttccacttctaaattcattcagagcatttatttaacacctactgtgtgcttcgaagtgtaccaggtacggggactcagaggtaaggactagtggcccctgatctcaaggtactggtggtagatagtgtgatgctcagcttaagggctgggcttctgaagtcggattaccactttctgaatgtgtggcttttcttgagtgacttcatctctaagtctcagtttccccatcagtaagataatagaagtaatagcagatacatacatagctcttagggcattgcagaatggaaggacctccttatatgaaacgcaaagcactgtgcctgatgcattgctagaactcaggcaatattagcatgttgtcattatcattatcatcatcatcatcttcaagacactgacaaaggagtcagctgtatgggaagagtgctgagacgctcttgtctccctggggatgaggtgggtgggtgggttaggaaaccttcacagagaaggagggtgatgtgagacttgtgtctgggagctgactcggaatttgccatctactatgttggaaaaggttctctgggcagaggtatccaaagttgccttgactatcaccctctgaggtcccagttgttgcctatatcatgtgaccagtgtgtggcttctcttgaattaagagctgcatgtctggactgcctgggattttacagatgtcatcttgttaactcttcctggagcttgtgacacccagaagatggcagtttatagaagccctgggaccttcttgaatgatgcttggtttggtttccatgctctgggaattcctcacaaggaaagatttgtcacatcttaaggaaggaaaaaaaggcaaatttgggagtccatggataccctattattttagattccaggacaaattgtcgaataagcacatttcataaaaacaatcctccgcagcatcccgtgacagcagctggtccctcaccacaggataattatgtctccttgtgcacacaaaagtctccgagggcatattgttgtggctggagtttctgataatttccaaattgaacaacctcagtcctaatgagtcagaagcttgtgcaatattttcaaacctcaggaacatctttttcattagttgtgcaataaagatagtaggcctatctctgtgatgagctgttttttttttttctcaaagtttgatgagattcgctgtagaattccttctcacatagtcttgggcaagattttacccgatcttccaacacatgagtcatatcatatcctgtgactaagaagagctgtctctttggtgccagttttgtaagcacagtcaccacttggtggagacggatggacacagttgggattgcccaggcagatgggcagtcttgccaagcagacataggggagggaaggctcaatgttcagcggtcacatctgcttttctgtggcagagtgagctatacaggaatattgtattctccaggacagttagggcagtgggaaatgtcaccaaacagaacagtgacccaaagagctgctgccactgggtgctctgtgggagctgggcactgtgctctttgtgttatgggccttgctttgttcttaacttgtagccacccagagaggtagggcattagccttgcttcctagctgagactacagaagaggctcctagaggttagctgtaatttgtccaaggtcagccagtgcaaggaggcagagccaggatttgagcccatgtctgtctcactcccaaactattcttcagatttctttaagtcaagtgttatttagaaatgttttgtttattcgtcaaatatttggtggatgtttccagctatctttcggttattaatttctagtttaattccattgtgggctgagaacatattttgtatgatttctattctattacatttgttagggggtattttctggtctagaatgtgatctgtcttggtgagtgttccctgtgtgcttgagaagaatgtgtgttctgtcgttgttggatggagtattctataaatgtcacttaggtctagtggattgatagtgccattcaggtcaactgtatccttcctgattttctgcctcctgatctatcagttcctgaaagagaagtgctgacgtctcctgagtctattctgaaacactggattgcggtctccatgatgaaccactagagttagaaaacctgagtcctagccccatttgggcctttgggatgactcccttccacctcagtttcctcaactacaacaggaggacgatgatgcttcccaggagacatcaacaggatactgtgacataagggatatgaaggagctttgtcaactcctaaagtttcaatgctaggaatcctaaagcattgaagtccaatgatataaggaatatgaaggagctttgtcaactcctaaagcttcagtactgggaatcctaaagcactgaagtccaatgatataaggaatatgaaggagctttgtcaatgcctaaaacttcagtgcttcaggagtcctaaagcattgaagctttaagagattaggacctctagttgacaattccagactcttccaggactcctgatagagccaacaccaagaatagtgaagccggaaggatgcaaatagtaatatgtctcctgggtgtcaaagtgtgggtctcctctgggcatgttctcttgtcctactgagacatgatagctcttggccaaagtgactgaacttgaccctctgtttcaggaaggccaaatgcagggttcaccactgtcatgtccaagggcagatgctttggtccagaacatcagcatcccagtcattataccaagcaagctgcaatctctgcctgcaccgtggagagcgcacgctcctcccagggtggcctgcatcctgtatcctgcatcctgtgttcttctcaggccgactttctgtttaatgtttgctggtcaggaaatggcctgagctgaggtttctcagatcccagcctgacctttctccaccagcatttttggctctgaaaaatatagcccagtgtggtttagccccactggatgaaacccaataggaaaagtctgataatagcagaggaggcgtaggaggaagggtgaggatttgagagcatctgggatggaccatgtgtgtggatattgttctgtctgtgggattgtgtgacacttctcatttacagtctgttcccttggaagtcccatcattggccaaacatatagtccttctgtcctctgaaaagtatcattctgctcctacctttgacaaccatctctgaccacatcaactccctgttttcatgcatcttgtggatgaggacaccaccttacctgtaaggacactggtggcttcccaaagccaccaactgacttgtagagaagacagaatcccagagtatgaagcctgagggtgaagggtcctggcaggtcctagagcccaacccttcacttcacaggtggggaaactgagggagccaatgggaacatgactctcacaagccacacagctcatctgtaggggccagtgtggagtctgtttatcttgagacccagggctgagtctttgagccctccccatctcagccacatcctcctgttggagcagttaggtgtttgggagaggccatggtccatactcatggtattcctgtaaagctggagaaacaggccttgctcccttagtctctctaatcaaaatgaggttgcagaaaacccttctccctacttctccctaaaataatttccttgggttagaagatgactaaaaagctattcatctgatgactgatgtctcccttcaagagttataagcacatataaatgcctttgaatggtaattataataattttgctgaagggaaaatatcagtataaatatcatggtggactcactgatgaatgaggactgaaatgctttcatgtcttttcagctgtggttagattttctttgagcagagtatacaagtttttcctctcctagcataaagactttttttttgtatcttttctctctactgtttagacatgacagaaaatgcatttatacatttgatgacatattgtactatctcagttctttaatattataaatgtaatttaattctatgaaaaattaagaaaagaagattcatatttcaccattaccatctctccagaaatactattattattattattattttgagacagagtcttgctctgttgcccaggctggagtcaggggcacgatcttggctcactgaaacctctacctcccaggttcaagcagttctcatgcctcagcctcctcagtagctgggattacaggcccacaccaccacacccagctaccttttatatttttaagtagagacagttttgccatgttggccaggctggtctcgaacacctggcctcaagtgattggcctgcttcggcctcccaaagtatgggaattacaggcatgagctactatgcctggcctaattccatcatttctgtcccaagtgttgccaccatttggttaactgttcccctgtccacatccatttaggccaaggttgcgatgttaaacaatcctgagatggacattttcatgtttatggctatttctgtatctagggtcattctcttaggagaggtactaagaagtacagaaactggaaagaaggatatggaatttttatggttctggtataaattgccaaattattttccagaaaggttgtagccatatttgttgacatcagctctagaatttcaacctcgtaagtcactgaaagaaattatcccaaaagcagtccttcaggaataatggaagaagatggtgccgaacccagccattctgctcactgttagattacttttttggtcttacaggttactttcattctcaggttgattgctcttaacagttgagcaatgtttggggtagaataatgagcacttttccaatttggttctacctggttgagttgtgatcacaggcagtctcacctgggaggggcttgggtggttgtcagcttgtccttccaacactcgcgtctcaggcgagcagcctgggaccagtgaggcgacctgagggctggaggtcacaaactaggaggtaacagagaacccaggtctcaggaagcccagtccagggctcgctgcagtaagcctctcggatgccagctctgtccaggatgcgggaggaggccagactgatttggtctgttttgaaaagtgatgaaaatatttattcaaatgttttgtacacataggcagaagtataacagaagctgcatatacaaaatcattttctagtagtcacattaaaaaagtaaaaagaaacaaagaacattatttttctttttaaaacagctttatcgagagataatttacatactataaaatttaccccaagtgtacaatttgctgttcttatgtattcacaatcatgcacctatcactaccaactccagaacactttcatcaccctaaaaagaaaccccgtatccattagtagccaccacgtacttctcctctgtccagccctaggcaaccaccggttcattttctgtttctatgaactggcttattctggacatttcatataaatggaatcaaacaatacgtaactggcttctgtgtcttagcataatgttttcaaggttgtccacgttgtagcagggatcattatttcattccattttatgattaaaaataggtctttttatggatacagggagaccagacttctattttatctcccctccctgatggggaatcctaatttcagcccggaaggtcactgtgaaagtctaaacgcacaggtgatactgactggttccattggaagaaactgtagcacctgactcaggaagccagcattaaaaccaagaatattctatacggatggggattacgcactgaaaggaaaacatgaggaaatgcacttttcagatttattagatcacagaacttctttggagctggaaaggatttcggaaaccgtctagcctaccccctcgtcttaccactgaggtaactgaggcccaggaaggggaagtggcttgttttgggtccgggaccacttttcatttcttatttgagccaaagcttccttctggtgtctgtctctgtttcacaagttcccgttgcatgggtgctgggtattgcttgaaaggactggcctcttccttgatacaggggctcgttcactgtcacctccctccctcacgtctcttgtgcccctctgcagccgcaggccctcctcctgcaccaggggggcacactcaacccgggtgggcactgcctcctagtctgcggccagaggctgggaggctggggagactgaacagccccggcagctccagacataacaacctatgttgaggagtcggtgcaggaagcgaacccagctgagaaatctgcgaaggtcaggaccggagccagacgcttatcaagaggaaagttaatggtgtttttgtgaactgagcagtcagctgtttccctgaagataataatagacacatcatgttgggcattcaggaggcatctaaaaaaaaattgtgcagtggaattgattggaagcttttccctaatacataaaataggccagaaaagactatcaaatgtaacagcaccgatcaaacccaagcactcaccatagatccaagcaaggactgaaaaacacgaattttttttttttttttttccgccagtgagtctgaaaagtgattttcaatgccaggcgcctttaaacacagacaacataaacaacaacatagttgttctggagaaggcatcttttcccggtaaagccaaagatgcagatctaggctgtgcttgtgactgacagcacagagaggggttcacagccagctggccaagtgccccccgaaagcgcatttcgaatctgctctatttgagagagactgtcttagccttgtttgggaaagtcttcctccttcacttcacctgccacagacttttccaggcaccatctgctgtagtcttggcccagtccctgcaacagttactgctgaaggcacccgggacatgcaagacgggggagcagcctgaggtctggcgtccggcaagcttttcccacttggagccgtctgggagactgtcccggaaacagaagggctgccaacacttggaagtgccaatgtggactgaaagttgaggacaggctccgggctcccccacctcttcctccttgattcattaaaaggaaagaaagaagccacacgaaactctcctgaatttcatttatttctatacaaaagacagagcgtggtcattcatcattcaaattttaacctttttagacaaataataattcctgcttgtgaattcagtgtattttaacaagaataggtctgagggccattggccatgggagacaccgaaggctggctttccttagatttgcagacagtggccctgatggtgcatagggtttcaggtttcctttagacctcagctggctgcctgtgccaccacttagcaatgccattgtctttcctgtgcattttctctgcagagttcgaggaaatccagtcgcgcaggcccctctgcccccatgtccccggcgccctggaatgtgcagtaccagcagcagcgattagaatgggggtctggtttcccggaatgtgcaaggtctcacttctgtttctgctgcctccatgccccagaccagtgctgggccgggctctgggctgcagccatggctgacaagtttccttggaatttaatggagcggggcagacagcatgcagccactcaaactgaaaacttgggaaagagatgtgtgttctggggcagctttgctgcattcgctgggccgtacatgcttctttttcctttccccaggcaacccctcttgcagacaggaggccccatctcctttcgcttcatgcctcattggccattaggaaccttttaaaattggtttctctcctgaccctctgagagaacatagtccaagttccctggaggaagaggaagcgctctgtttctctgcaattcacggctcatttaaatgcagcccacgtgctgtctctccccactcctctgcctgctccccttgtgcttctcatgatcattctcaaatttagtgagaaacctcacaaagggagtttttcttagggaaaagtcatccttggcctcctgaacgtggaccagcccctctccccagctgcacagcatcaggttagttaaccacctgcctccatctgggtcctgtctggacaggcctactcacacctgctgcaggcgtccgacttgccctcaggtgcctgtggctggttcagaggggtggagcccacattccagtcctgacagctaaagttcagcgagaggaccctgcattcagtgtaaagatcaatattccaggtcctctcttcctgccacccagagactggccgtttgcaggcactcggtcccagttgccctgggcctgcagcccttgcattctctctgctttgtctctgctgttgcacccctgccccatcacagatgcaggttgggggaccttccgctgggaagtgagaggctgggaagtaagaggagcactagagggatggttgagctcgcatccagccttgactgcattcgctctcccccacctctctgtaaaggtgctgagctgtgagtggaaccaagtggatgagagtggccccgggcacctgccgataagtttcccggtgtgtcattttctcctgggagtcccatctggatttggttctggatttatttattcagcaagtagcctctttgtagttacttttaatctagccatgctcggggctgaaggggatgccaaagaaatatacgatgagcccctcagacagcataaaggtgaagatgaggcctccagcatgtaccccccaacatataccccaggaaattctgggtgtgactggattttggacctaccaaaagctgctggtgcctggaggatggggccccgaggctggacctcactcctgctgggttactgggctgggaaagtactgatggcagctgaggagtgtgtcccagacttcactgagccattcccaaagattatttcaagttctcctgaccccgcactggaggcctgcggtgctggccttctttatttacagtttctgactggtgtctagcagccttgccagagagagtggcagtgtgtctgcaggcgaccaggagaaatgtcccaggctttagggcaggactgagcatatagcggtgggggcccagcaggcagtctcctggacagttacttctccttgtccttacatggtcgggaggttgctgcctggcttttcaagcgaggatggaacgtgctatccatgggccttaatttccaacttctgcatgatgcattttgtgctcttgcctttgaaaaaacgtttttattttcttgtcactgatgcccaaacccacatggcagaaggaagggaggctgggacaggggaggcgatgagctgccgctgacggacctgcccagtttcttagctcatcccggcctccatcctggtgagcagacactggcccaatccagccatatttttggctgagtttctgtcttcacatctcatcctttccctgggatcctggcaattgttggtactgggttgtattcttatttgtaatctttaaagtaggagtacctttgctggtatttaaagtggaggaaatcaggtgaagagtcacaagtgatttgcaagctgggagagacattagaatgtaaatgtgaggaagcgtcagcatgaggggcttgcctgggctgcacagcttgccttgcctggagcatgcactgttctggcattgcagggaggatggctaccttgcctccctgcaggtgggggactgtgtcagcccctgcggactgctcctgggctcctgggtttgaccagattaaggcagcatctccagtagcaccggagcagctcctgagacgcttttctgtgctaaatctggattttgggtattaaatcaaatgaatttgtaatgcagtcacacattgccctgtgttcagaagggtgccgcacctgttttaatgctctgctattgctcccttgggagtcttaataatttttgaacaaagggccccacatactcatttcgcactgggcactgcatattatgtagctagtcttgaatctaggacagtgcattaaaatgccattgattggatcaatctgctcttacaactgatttgaattttgggaacatgctgttccctgtgaataaaggaggattcatttcttttccctcgaatacactgcgttctgttttccaaattagctctacttatcaactctgctgagaaattggaaggcgggattgttctggctggaagggaaggttagattgttaatcctgcgtcctggccctgatctcacaaagtgtgaagcatgttcccacaatgatgtgggctgcagggggctggaggctggctgagaaggtggggaccaaggagggaggccagcctgggagccagacagatggggtcaggctctcgcttttgccactcgccagctctgaggctttgggcaacatgatttaattctctgatccttgtttttttcatctttctgtagactggtgataagatgcaccctgcaggcttgcaggaaaaattagagataacatttgtgcctattattgggcttgacatatagtagatgctatacaataaataggtcctgttattcttattgataatattattttattgtcaacattgaaggttgggtgggatttgactagctgcgggggaggagaatgagatcatccaggccggaaggaaaagaggcatgaatgcagggggatggggtgaaacactttggaggtgtggggagaggtctgcagggtgggagtgtgcattaaggagttctggggagagtggaggcatcagtgccacatggcaaatgagagggaatcgtgggcccgaggagatggagatggctgtggggatccggcaggaagtttatgtgccccaaagtggcattgtcagttagggggagacactgaagacagaggtgaggcctgcctgaattagcgcagagtggcattcttggaaacttcagaagcttgagaagagccacttggaggtgttgaaatgtacctgggagggatgtggggacctggctctggtctgagagctgggagacggtaacccaggtggccttggccttgaagatggggcatgatatttagtgctttatgtgcagtctcacctaggactcccaagccctgtggagtaggtgatattagctccgtgttacagaaagggagactgaggctcaagcagggacaggcacggtctgaagtcacacagctgtaaggggcagaagtgggcatggaggcattaacttagagccgaaaggtgtgaccttccttagggtggctggccccacggggaatgtgtgtgggttggagtacaatttggtgttcccacccatcccagatgctctgcgtttatgaacccaagtttccacatcagggcaggcgagggcaggaagctctacagggagaagggacaagggacagagccaagaatgggggcagggccccagggtcccgtgcagggacaatgaagggagttggcacacgtgggttagctgctggacagtgtggggagagagctggcctgggagtctaatgggaatgccagggaaagctgccttggtcccctaaagtgaagcccccatgctggccacggagtgttggtgactgagggtccctgctagctgtctggccaaggcagtgtgtcctataggtgtagctctggtgtcctgctggcatggcgtgagtgcccctcatgctgagagccagccctgtgctctggagggaggtggtgggaggaggagggacagtaggaaattgccacctgagcaggaattggcaccttctcccactggcaggtccaggttttatggaatctgaaacttgtacaattcaggatactctcttcaagaaaaaaaaaaaaaacccttaaattatgaatataacattagggatgaaactattatttatatagattgaaaagagaaaatgcccaaaatgacaaacttcagaaaatataccaatactgcaaacatcacaaaatccagaaaaacaagattaaaaaaagctaactgctgaacactccttcatcttgaaaatgtccctgtctcctcctctattttttggctgtgaactctgctcaccttttcacatgacaatgcttttgtaatatttcctaaagagaaaatagaataatttattattacttttattattttttggattattgttatgatcaagtcaatatttttctgctacccacacactcactgtcttctgtacaacctctggcctgcaccaggggaaccagcagggtgagcagtagggtgtccctggagaccacacatatagcaggatagacacagcaatttaactagacacagaagggacttcaaagcacacaaatgtatctcatttaacccaaacaaaatgattatccagttttacttttcccttagcctcttcccccaaatgccggcagccaccctgatgggatagatgtgtgacagagggcaggagaccgtggcctcaaccagctgcagcttcactctttcaattctacatactctctacaagccgtgatgatagcactttgctagggcccctcacagggcagatggagggctccatgctgaagctttgtggatgtttgctgtctatccacttctgctccttgtgcctatgcagggattcaggcccaaccactgcagagagcccaagagcatcaggctcccaaactgtcatggttggtggcacctttagtagttgatacggtttggttgtgtcctcacccaaatctcatcttgaattcctacatgttgtgggagggacctggtgggaggtaattgaatcatgggggcaggtctttcctgcactgttctcatgatagtgaataagtctcccaagatctgatggctttgtaaaggagagtttccctgcacaagctctctctgccttctgccatccatgtaagatgtgacttgctcctccttgccttctgtcatgattgtgaggcttccccagccacgtggaactgtaagtccaattaaacctctttcttttgtaaattgcccagtctcaggtatgtctttatcagcagtgtgaaaatggacgaatacagtagtgcagtcatttcttcatggtcctcagtaaggccaaaaaatacccaacagttccgttgatcaatcagtgaggtccaaacaatttgataagtatttgtgtccctacaacacagtggtcattaaaaaaagacattttaatttcattattcaataagcatgattacttatgaatgggatgtgtgcacctgttgggtgtcacatgacctttcaaatcttggaatcagtttggacaccaccatccccatttccagttcaacactgatttttgtgtggtacattctttttgtcacagtgactgccagaaatccaacttcatatggactcatgaaaagagatgtagcgtgatctgatttcaaaactatgattgatctagagttagtttacaaggtgtctaacagtgatcccgtatcactgtatttccccagaaaacctgaaatatcgatgaattttctgtggtattctggggtcccttggggcagactatgggaaccatggcattagaaccataaggacacgattctggcttcttcctgcctcagatccagtctttacctggcatttttgccttaaagatgaaagcagcatacattttgatgtatctaaagcacatattcggccaggcatggtggctgacacctgtagtcccagcattttgggtgaggcgggcagatcacaaggtcggaagttcgagaccagcctgaccaacatggtgaaaccccgtctctactgaaaatacagaaaatagctgggtgtggtggtgggtgtctgtaatcccagctgctgaggaggctgaggcaggagaatcacttgaacccaggaggcagaggttgcagtgagccgagattgcaccactgcactccagcctgggggacacagccagattctgcctcaaaaaaaaaaaagcacatattccactttgtgcttattcttttgagagaaacacagataaaagtctatcctttaattcatactccccatactgtgattttcatttttactgcaacaaattgtgttaagtgtgataatgaatgtcaaacacttaatgccttgctcttttcagtaacatgaaatattggagaataatgactgaagcttacctgcactgcgtatgtctcttttcttcctccttgaaggaagttgttgaaagttgttaagaagtattatgtgtaaaactctagggatgatgtgctttaaggaagcaacatttatgaagttgtgtgcttgactagtagtttataaagagggaagacgaatcatttattatattgggattgaatcctggcaatttttaaactataaagttacaggaaatgttggctactcttaatgggccatttattgtgttaaatatcagcaatgataaatatttactaggtaagtggaaagatccatctctataagttgttgtaacttaccattttacgaatcttagttactcagtttttctgtttaaaaatgaaatcatgtagcactgtataagtcattcagttttttcttttggagaattactctggattgtctaggctctgtgttctccacatatattttagaaatagtttgtgaatttctacaaaaaatcctgctcggaattttcactgggagtatgcttaatctatgggtcaatttgtgagaaattgatagcttaacaatagcgaatcttctgatccacaagtgtggtatttctctccatttatttaggtcttctttattttgatagcattttgtagctttcagtgtacagatcttgcaaatatcttgttaaatatttccctaattattcgatatttatttttgatgctgttatagttatattttaaaaattttgattccaattattgctaatacatagaaatgcaattatttattgacctgttatcctgtgacattgacaaacacagtcatatattcgtagatttctagaatttttctacatagactatcatatatatcatctgcaaataaagacagttttacattttcctttccaatctcgatgccttttctttctttctcatgcctcattgtgtggtccattactgaacggcagccagttccagctttctgttcaataaaggagcagttaaaagggccaggccttgaccttgctggaggcttcccatcctcattgccttctgcttcctcagttctggcttaacagaacagtgtggggaggaggcatgatccttacctactagggcgttacaatggccttcttcaggttggttgattcatcaggtttaagcgctcacctgggctgcagtcaggctagattatctgctgaccttgccctgtctcctttctgtagtggggtacccttgtaagctagggagaagagatacaggtgaaggccggaaaaaccagcctgccacacagcttccctggatcataccttcgcagtgatatgacgacactgttaggaggagcggaggtggctgagtgggtctccagacacctccctttacctctctgctgtgccactgatgtgtgacgtgcttgcacctatacagagctgccactgagcagcaccgtggccagtcctgtggattttcttctttctaaattgtatgccatggcttgatcaaacatttcatatacagtagatcatgaaatcagcatagaaaacacattgaggtagatggtgttaccacattttatggatgaggggctaacacttggagaagtgaggtaacacgtccaaggccacacagctagtgagcaccatgctgagggtcacactctggtccatctgaggccagagactgtgcacagccttctcctcatgctgagtggcctggacacccccaccctctttcccctgaaccccttggagagtgggcagtggcagaaccaacctgggcccatctatggggattctccattgggattgacccgtctggaaggaagacagttgacccacagttaagatcacagcagatgggccagccagggtttctgtagaacatcaggcagtggccactccatctagtttcatggatgagcctttttaatagaacaggaatctaacactgaaccaagctgcttttagacacacttttattcctcactctgaaatggcatttggacaagccaaatatttcttcttctttcagttgacattttgtccatctttgaacagttagctgatgtttcttctgtttagttatttctgttctattttcctgttgccactggtccacccagggatggtaagaatggaagtcaatggttgctttttcatctgggatgcgtcacgaaggctcagtcaggcttgtcatatggtctgtgctcccactgctccttctttctgtttcctcatctacagaatttggagagtcctggacctgatctcaaatttcacatgttctttatcttcctgcagcacgctggggagagggagagacagggattccatcacagaaggttggagctggagcagacttcacagctcattctagaggcatttggtccatcttcacagctcattctagaggcatttggtccatcttcacagctcattctagaggcatttggtccatcttcacagctcattctagaggcatttggtccatcttacagatgaggaaatggaggctgcccaggggactgaggctggaactgggccttccagtggccaggccagatcctccttggtctcccttgttgctttcctggtgggcagaccctggagccactttctgtgactgtgtgagaaggcgactgcccagcaaaatccatcttcaatccatcttcatttttgcctctggcgtgggcagattctcccatacctaattcgggaagccagaaagaggaagtcagttaatgatccttagtgggaaggtgctagtaatggtccttctcgtgagtttctgaaacaccacgccgtctctgtgttgctggcccggccggagttaaacctcttcttggcctttccccaggaagctggtctgaggaagcccagatgcgtttgtttacagctgtctctggtgacgttcgccaggctctgtgttcagaaggaacatttccattcccttatttacacctcccactggagtgctcgaggagacacaccaattatttccaactacctagaaacctgggagggtagcagatctgtagggggccggtgttgaagcgagaagctgtaaatctggtgacactgtgggcttgggagggcttgcccggatctacctgttacttatactctctattaagaaattttagtgtccatggagaagttatttaaagtctgcgagcctcagtttccccatatataatatgggaaggatacctgattttcctgttccacaggaaggtagaaaaaattaaattaaggcaactgatgaaagggttttgaaagcaaaaataataatatgatactgtcctgaatttgttaaattattcctcctagtagttgcggatctttttctgtaccttagaaaaccatgctatgtaaaaagagatggttccagtctttaaataaagcagctcagaggtcaggggccaggacagaagggggccctttgttcacagatgcgctttcacttctgagaaagcaagtgtgggagaggcaggtggtcctccagatgtccctgtgccccatggtgtcaagttgggttactatggccccttgtgacccagcgtggtagggatgtgggagccagtgggtatggaactgtgatgggtcacaagagggctgggacgtctcacagcttctacttacagcctagagcctggggaagggctgccaccttagtggtaagagaggcatgtatgtgagtgtgtgtgtgtgtgtgtgtgcatttgtatgtatatatgtgtgactctgtgtgtatgtgcacatctgtgagtatatgaattgtgtggaagtgtgtataggtgtttatgtgacagtctgtgtatgagtgtgggtgtatgtgtgtgggtgtgtttatgtgtgtacgtgtgtgggtgtgtatgcatagtgtgtatgtgtgagtttgtgtgtgtgtgcctgtgcatctctgtgtgtatatgcatgtgtgttaggggcaggcacacaggcctgttggtaaatgagacacaaaatacctacaaaatacaaaatatgagacaggaaatacaagccacagttattcatttttcaacgcaacagacataagattaccatgtgaaattgctctgaaagtttccaaaagcttcctgtcaattcgtagagagcagctaacaaaggagtgcgggtccctggagcctgcttgtgcagcattgagctattccaagggggaagaatggggtgcatggctcttagctgcagaccagcctagaagccctccagcctgcttgagcagacttgttaagaggtagcagcaggtggcagagattaggagctggagtagtaggctaagggtgcacttccagggacacactgcctctgccaccacccgtgccaccaaaatgggagcccagaaccctgaatctctagcagcctgtttctgaatcagttaccttgggtgtgcgcctctggtcgacagaaactaacttttagccctcctgggtgagagcctcacatcgggacatgtgacagctttgttgaaagtagctttggaaacgcccaccacgtggggccactcactgtagtataaacggtcatgcaccactgagtgacagggatacgttctgagaaatgcatcgttaggcgatttcatcactgtgggaatgttacagagtgcgcctatcaaacctagatgccatagcccactacacacctaggccagatggtagagcctgttgtttctaggctgcatgcctgtacagtaggttactgtactgaatactgtaggcggttgtaacaatggtgagtatttgcgtatccaaacatagaaaaggtacagtaaaaacaatggcgttatggtccacggttggctgaaatgttatgtggtgcatgactgtaggtataaagcattatggtcgtttgattttcctctttttctcacccacagtcttaaggcacctcttatgccttttgtctgggatgtcccgggcagggttggaacatgtggttaaggcatggtggaaactgctttggggacggacgatggcctcagcttgccttggggtgtcagtgggaaagataggagctgcccctttgccttcatgtttcttcgtaataatctcagatctacccatctggtgagcctctcctagagaaaagccccggtgctccttcgctcctgcggtgtttctcaggagggttgcttctttgtaatggtggggactcagggaagggacgcaggcagagggtgataccacatcacaaagggacccttggctgggtgcggtggctcatgcctataatcctagcactttgagaggctgaggcaggtggatcacctgaggtcaggagttcgagaccagcctggccaacatggtgaaactctgtctctactaaaaatacaaaaattagtcaggcatggtggtgggtgcctgtaatcccagctactcagtaggctgaggcagaagaatcgcttgaacccgggaggtggaggttgcagtgagccaagattgcaccattgcgctccagcctgggcaacagagcgtgactccatctcaaaaagaaaacaaacaaacaaaaacacaaacaaacaacaacaaaaaatacttgggccatcagcttcttggaaaggctggtgtgaggtagaagcatttgctggtgcctctgctcgacaccagagcagaggtgattttttggtgactctgttgagagcagagaacctgagcaaagaggttatcatgagtggattttactgccttacttgggtgggcattcccttgggagttcgatggacatttgcagctgagcccaggcaggggaactgtgctcactccgccttcagaattccaaaggctgagcatgcattttggcttcctctaacccatgtctttctctaggtgaccacagcagagtatcattaagtatctattctttgcttttgttctcagggcaggaagatcccaatagtttgcgccataaatataactttattgcggacgtggtggagaagatcgcccctgccgtggttcatattgaattgtttcgcaagtaaagagagccttcctttttcctataacctctgaagctttcaccgccactagcaaaacatgagagctctttttgagacacattaaagtgtcaaagtgtcactgaatatcttcctactttaagataagtgtgtctcccttcaaacatttgccctattcgactctatgaatctacagtcttaacccttctaaatgtttaaagaacctcgggctctgaagagattccctaagaatattttgtaagtgaaattgtttgatgcatgcaaaaaattggcagattgtttagtttttaaatgttaagcccaatatataaagaagcgattgctaggtgtgtgttgctgttgcagaacccattcattaatcaatgtgttgaagcgttcattttaaggtgttgcaggcttaagtgtgtacttctttggattttaggcttccgttttctaaacgagaggtgccggtggctagtgggtctgggtttattgtgtcggaagatggactgatcgtgacaaatgcccacgtggtgaccaacaagcaccgggtcaaagttgagctgaagaatggtgccacctatgaagccaaaatcaaggatgtggatgagaaagcagacattgcactgatcaaaattgaccaccaggtgagtatgttttcgcctgcagaggtgagttctcagatgccctggaacacccttggcaaaggcaccagagctctctgattgcaggtgattctcagggggcactgaagccagtctaaaccagtcacaggagggccttgaggagatgctgagtatggcctgggcgtgtgggagaggcaggggctcaggagagcttctgtaaggagccagataaaagtttttaaaataatgttttaaatgtttatcaaagaaagcaatagatttgtaaagaaattagtaggtaagttgtgaaaattgagtctccttcccattcccgatcctgtggcaacccttgttacagattttatttatcctccacagatacgtcatgcattcacagtgaacatagaatttactggggtttagactgagccatccttaacttgtcaacagttactctgaaaacaaaccagctctcccaaattggggttttgcagggtaatgaggtgtgtttcagaacaatattccatactttatatatcttggaaaccttgagttaaaacagagctaatggatttcttcttcccagaccttctcagagcttttagtatgctagtgtgcacgtggcttgcctacaaaagggtgttgactgaactatttgcccaaattataatcatttgagtatacagcttttttttggaggggggaggggcagaactgagccataccaagatcaatctggcaaatgctgtatttgaaaatgctttctatttaaatattctctttgcaatcatttttgctgttgaattgcttagcaaagtcttcatgtctgggacaatatccatttcttactgactcatcaaaaacccccactcgacactttgatgagagaggttttatttgctgtgtggcatgttcagtgaaagcgtggtttctagtttcttcacatccttgtaattttctggacttcagacggagggaacaatcagaggaggttggaatcctgcctctggccaaggaaaagaccagagactgagccagttggggtctcttgtccagccctctgcttgcctccctttacctgggtgtgggctgagtaattccagacaagcgtagaattaatcaggctatttgcgctgttggatggcatgctgggtacatctccttctggaaacagctctgcgtgtgctgtttgggtggtaggattctgggtctcctctgtctttttatggcatcaagttgctgcccagcccaggctcctttacggccagtcttcagaaaaccaccagctaacacatttacaaccctccttccccgatgttcctataacctctctatggccgggtggccaggcacggccaaagaggctcagggtagatatagggtctgtgtccggtgtgtgtaactggccttgagtgaggctgcagttgtgtgttatttctattaggtcactgtggaatttctagcaacaactaatctttcaaagtgtgtttattggtcacaggatcattgggccagcctctgccttcgttctttttcacctaatctgcataatagctgtattatccccattttagagaagaagaaacagggactcagagaagtctagtaacctgtctgagaccacacagcaaacacgtcatgaccctgccctcctaaggcagccaggctactgctcccaacgtgtccaagcccatggctattgttggagggatacaggctggccccatggaatgatgggacagcttgaccttaaacagcccatggaaaggtgggtgcatctggtttaggaacaggctgctagaaaggtatccaggatgtggtagtctcaccggaaggagccagtcagaatagcacagcctgtggccacgcgtggggcctgttcagcctcacagagcctttgggaggcagccagcagcagggcatgagctgtgtgcaggcaaggcgctggcctggacgccgcccccactgagtaacttcgtgtttggaatgcgtgggcacataccgtgcagctgcttctggccggcggatattcttttccaattttgagccaaggtggagactgtctcctcgtgtcatccctggcatgtcctggcaagacacgaacgatctcaatagacaagctttgcagagtgtgtctgacctgactcctgctgtcctgggagctgagctcttcagccagcagcatgctgtttgacatgtgtttcaagtcccccaagaaagggtgcttgaaatttaaaattgaactgatgtggcttttctaaatggaattggaaatgaaaggatattaaattgcagacaaccacacaaaagactggtttccactgactaaactgcttttttttgctgatagtagttggaagtagggagagtaacagcatctcttccagctctttctcttttgttcccttgttttgatgatgggttatttcgggggaggctctggctggccttgctttgtgtcaccttagggataacaaagaggatgaaagagatcaggaaaacagagaaggcagaacagaaccagcagaaactgtgcttgaggaatgaaaatcacctacatggctccttgtcgtatgagactgtggcccaacctcccccaaagccacttaagagtaacccagtgaagctggtgagactgcctgccgcgtccatgggcccagtgactagcttggtggcttatcatctggacccagctcctcccctggcatcctgatttcacttggagggtcctccattgtccttcataaacgtgtttactttatttttttttattttttgagacagagttttactgttgcctaggctggagtgcagtggtgcaatctccgctcactgcaacatccacctccagggctcaagtgattttcctgcctcagcctcctgagtgactgggaccacaggcacgcaccaccatgactggctgatttttgtatttttagtagagacagggttttgccatgttggccaggctggtctcaaactcctgacctcaggtgatccacctgcctcagcttcccaaggtgctgggattacaggtgtgagccactgtgcgtggctataaatgtgatattcttgagactttcagtgaaataaaaattaccatggacacctgtggtcattgtccacttgccacccacctaccccccttactggcagcagcagccagcatttcacatctccgtcatcggacagcgtaggtgggcccatcagtcatggtgtcctaccctctggtgccaaggagcggacacatgaccaagttagggcaagcagaggctccccctggaactgcaaagtgaagccggatgtcacccacagagactaacatggtgaagctgctgtaggccctgctcttgagaccccagcactgtctgagttcttgcactttctgagtccagtttcatatctgcttttcctcccgttcttggagctcccctcacatctccagtggcttgaagttgccagagatgtttctgggcttgtgaccaaatgactccttttctgcttctcactgctgagcagacacatgtgcgctcactttgcctgctgagtcttgggacccggaagagctcttgggagacgctcacggagcagccccctcttgccggccctgctgactccctccaagcaggaggggagaagccctggctgggcatcccttaatgtgcttctgcccaaatctgaaactcctctttcctcgggacccacgaccgtggccagcctgcctggggagggaatcccagctgcagaaagtcgggacagtatgcgtgtaaacatgttaatagaaagcagctttgagggcaaactagttcagctttagttacaaactctttccaaatgtgtttgacatgagccactgccagtgtgcagcatatgtcaagctttcatccaatggtggcattttgtcccaacgggtttttttttttcctgagcagtttggggcaggggtggggagagggagagagaaaagtaaaaagagagcagtttggtttcttcaggctggagtacaaggtagagataatgggatgtgttgaagaaagtaggagggaaagttactttagttacagctgtttgtccagctgtgctgattaagaaacttggagaaaagcatctctggaatcatgtccttcccatcttgtatatagcctttgcagatctcctgcggttctgagagagatctgaactgcttaccagggccttgagggccccatctgattgggcaccctccctccctctggccctcctcctcttcccctcctcccctcctttctctgcccccacctgctctgctcagacaccccctgctcggttacttcccacaggccagggctgtcccctggggccttggctgttcccctcctaggagcacccctctccagctcctcatggagccaaccttcccatccttcaggcctctgattaaattctgccttagacatctctccccaccccactgtgggaggtgacgccccatgccccagtctcctcaatcccaccgcgtcactctggggacacatcaccccagggacaactgcattccactcttggtttttccctccttgtctattgatcacaatttagagtcgcctcactcatttctcagtcatttgtcaaatgaagtccatttctgccgctagactgcggggttggggacacatccggctgatcggtcctcaggtaggaggtgcttggcaactttgtcccgagtaggacgttcacagctgtctgccctggaggaagcaagggcacccaccacgtggatggaattgaggggaaggcacccgcggctcctgcatcgagcttccgtcctatattcaatgaggaaatgaccctgcagcaggctggctgcagatgcccctgccatcccgctttgcctgcctggagtttgatggacatgtggtcctgtcagggctgcagcaggtctgtggtctttggtaatgcaaagcgctggggaaacagtgagctttcctgtgggtgcttttctctgacgccaacaaccaggtaaatatttggaaacggccttgttgaggcttgtgaggtggttttcctccctcccctgtaggcctgcgccacccctccaaccccccggccaccttcaggccagatggcacccacagacctgtttgaagtggctggacagggagccctctgggcgctggggccgctgtgtttgcagagggtcctcttactgctgagctggctggtgcagcggggaggccaacacccctgatcctcatcaagttcagaggggagtcaccgcgggtgaggggcctggggccttttacatgtcctgggagctgctgggcaggccgctcttctccaggccaccagaacttggccctgcatgtggcgaatctttcctgagtcagctgagtgaggggggttcaggcagccccccgggacgtggcagtggttggggatgggagtgggctggtgcgtgccatgactcacgccggttctcctcaggcaagctgatggtcagacgtgctgactcagtggcctgagctcgtccaaaagtgaatcagagaacgcagggcctgggctcacccactgccctctcctggagtcatctgtcactcatcctcatgaaggaagcgcctgggagcctggaatgcacttcgcactgccccagctcccctcttgtttctgtgtttttccattttggattctttccccccactccttctgtactgggcattttgtggtctcttctttttctccgagaactctgagggctaccattgcatttgctaatgatgccacagacggtgttgatgttatgaggcttctattactgtattgattgttaccatttttagggggacaggaatcaatatttcatgagggaatgtgaagccagacagtaaagtagaagctggcttttattttgtgccaggctttgtccagaggcgggtggggacgtggctcctcagctcttgactgcagctccttctggcatgggaaacgcttcagttccccaaactctcagagctggagaccctgtgtgttctctggcccggattcaagaacttagttgattgtcaaggaaattctttggctatatttttctcttaatatggtaatggcttttttcactctggcactctcttttcagggaatcggattaagactattatttatggttctgaaaaagcagttcccaagttggtgggactggatttgtttaggaatgtctcctgtcctcttcattgaggggggaatacaaattggttccatttgacagtttatcaagtgtgtgacagagtattagagtccagggttggccaactacagccagtagtccaaagctggccctctatctgttgttgtaaataaagttttattgggacctggtcatgttcacttatttaggtagagtctatggctgctttcattctgcaccagcagagttaaatagttgggatgaagaccacatggcccatgaagtcaaaaatatttgcttcctggccctttataggaaaaaattgccagccccagtggtaggcaatttacaccttgtcctagaggagctgaaagtggctggaggcaggaatgctcataagaaccaagcgaggtgaagcactaggtagctgcggggagcggaagagaagctgattagctgattttgtttgccctttcttttccagagattgtgggtttttttttttttttgcagagatgaagctttggtcttgccacaatagcagagggaggccttatttttgtccatttctctatgacattggtagaaaggagtttgtcagaattccaagctatttggcaattatccaattttgagatcctaatggatctttcaaggtctagtttgttcattcttttagtgattccttattaattccctgattttatacatatgtgttgaacatctgtcttggccaaatacttgttaagtgctgaggatgcagccacagtgggcaaagccatgaggcttaagatctagtgtgggaaacgggtgaagtaaagtaaatatggcaataagtacagtgcatgaagcaaacaggtgaaggggtagaaggcctcgggctgcaaagatagtagatagtgtaagcagggaatcttatctgaggggtgacatctaggctgagatggaaaggacagtgagagccagccaaggaaacaagctgggtgacaagagttgcaggtggagttgcttaatttcccatttctgctcagcctgcagaacctagatcttggactaattgcaaactgtcatttccttgtgagtttattagaaccctccagaacaagtttctggttagctagtttctctgtgtgttgctcatttcttgttggttctggttctttggggttcctactcatactccggaaagctccaatgtcttaagtagtcagtctcccaagagtctgaaagcacaaagattcacaatgatacgatcacctctcagtcatagcagcatcgatgcagttccgtagctggtttcctaaagccatccagacctctttctgtggcaagagagaaataagaccttctggtgaactgaggactaattatcctaataaacatgtgaattaacagttcctttggttaaacaaagcaccagaatctgataatgggaacatgtgactcacggtatttccctctttgctttatctaccaggcagctcacgaaaccactggccttccctgtgttcccattttatgtcataaatatatgtttaattaacttattataaaaggccctttgtcatggaccatatcaaattattcttatatagaagaggttatacatgttttaaacattttaaaataaatctgaaaagaatactacatcctgggcaacttccccgcatatggggctcaaagaagctctatgtggttatgggtaaggcggagtcagagtgccttcagtgtagttcagcagatgctgagaggctgctgtgtgctggactctgatcccactaaatagagtagggctgagcccctgcccaccatgacagcctggagatacaagctgttccctttgcctccctgagccctgagctttatagcctatagacagctgaaaagcaggctgcatcggttaccccgtcagttacccagacccaaatgccaggccttggctaaccccagttattacctaatttcaagatcctaatgtatcttttaagacctggcttgttcattctttcatttatttacttactcattgattttgtaaatatttatggagcatctgccgtgctacatgctgttgtagcagcatcagccaccctgaagttggtggatgaaaggggacagatcaaaggggctgatgtatggaggagacacaagttagacttgaccaagacaatcttattcctcctctggatgccacgaatatatacagtcattagctgttgggcccccatgaagactgttgacattttgtggtttaaacactgaagagtaagggaatgttggaaatggcaaacatctgatatagtgtaaagaagactaaatattttggtggtgttcataaacactgaggaggaaagtcgtttcattttgttcatttgtgtgctctctctctctctctgttatggcacattatcctctgttctccttctccttttctttttccttttttctcccttcatctccccacttctctgatctctcccacctgaaccgcttctaccctgctgccctcccatccatcctacctcctctacttccctccctagacagtagtaatcacatgtcagttggagaaacatgatggcgacttggtcacaccgttcttctcagtctgtatatgttggtgatctccgtgcccatctggtagatccctccttccctggctcttctgctcaccacaaccacccttgactttgtgatcgctgataaccttcaccttctctaatctgaatcccaagcttctcagtcctggcccaccacctcccctcctcatccactccgaaccctgaacggaagctgaatggaaccctgaacggaagggttctgaagctgttgagaaccctgaacggaagctgaaatatcaatgggtcattgcttttcacagtcctctgtgaaagattactggccaagccagcatctggagaattcctggtctaccgcctccctgtctggagaagctggaagagccagctgcatagggcatgtgacccatgtactcacaggccctgtgccctgagctcactgttttaattttatctttgaatttgtatttttgtgaataaagtcctatgagctaatggagcatgctcagagaacttggggatttagttcaggctggattcctcctactgcctccccaatccctggtcccctgagacctccagccccacctgaccttcccttccctgtttctatgcagcgatcattgctaccctccatccctggaaggggtataggcacagggcagttctaggttccaacttgggcaccgcataacatcttagtggtgcagggttcaggctgatgatgccatggtggttctgtgggctactgggcagggtcaagccgctctcaccctgatccaggtacctaatgcaccctgacacagaagtggcagtgtccttggggtcatccattatccatgtgttggaggagtgggcccttagggaagatgcttggctcaacttccccacccctagccagggcacgatccgaggtccaggggttggtgggcacgaggccaagtcgtgaggcctccagtgtctgcactcactgtcccgtaaataaccacaacaataactagcaaaccaaaaccagtgtgataggttgagagagacagaatgtggaagaagggaaaaagctttatattttagtacctttaacagtgctttctgtatgctttatgaacaaggagcctgcatttgcattttgcactgggctctgctaattttgttgctggtcctgctccctagtagcccgagtcagcaaatctttggttcatctgagtccacagtgcattgacccgccctttttcacagttcctcccctgcccatgtgctcacttccctccttacccagcttggctcactctctcaagcaagtctttggatgctgacatcccccctaaacaacccttctgcggcctggtttgattgtccttaggaggcgtgcaagttctatggcactgcttcttgctgggtatagaggatgtgctattttgtccattgcatattttttaaagaaaatgaaaggttagcataactgtttccagaaggcacattgaatcactcagttgagtcccagccagttgctgcagcgttagcctttgaagcaaacttgaaccaacacaggaccagcctggaagtcccagcctccggaaacgatgcagtggattctgcagattcagcaacaaaaatatttttgtaactcaggaacacttcgtaattttcaaaggcgagaaagaagtaattgacttggcttattaggttgaaaaagagttgccaattttttctttggttttgttgttattgttttttgttttttttcttttctccaagcttcagggaatgagattaaatgagcactgaagtgctactaggcagaacctgaatggaaggaagctgaaatactgatgggtcattgcttttcacagtcctctatgaaagattactggccaagccagcatctggagaattctaggaatcccccctcctcttgcagcggtataagtttgcgggaatcatctcaccccactggggagttgtatgaaaaaagggatttattagggaccctgttgcctgtttggatcttaccaatttaactattgtctgctaatggatgttttggaaagcaaccaggttttctgtaaagaacagctaattgtcagagctgagatgaccatgggagatcactgggctcaactcctaattttagaggtggtaaaaccgcaacccagagaagctgatcaagtgggccaaggtcgtagactgagttcatacaggaccaagacccagccctgatgtcctgctatctgggacagtgttctcccggcacacgtggagcctgagggggtaatgtgtgtgtgtgtgtgtgtgtgtgtgtgtgtgtatgtacatgtactcatatacacataggtgttttgcctaggttttcacttctgccccaccttggttgatcttggagaatgagcccgaggcgcaggtgcgctgtcagcctgggggcttcactcagcacaggcccaacttttctgctctgggggagttccagcagttatggttcatctgtggttcagttatggaacccacaccacacgtagcgcccccaaagccgaggctgcatgcacagacctcccctcccttctcgtggtgggcccctgcttggattcttcccaaacttctcctttgccctgctctgtgttatacccactctggtcccctgtccctgtggagtgatccagggcacaaggacagctgtttcactgctggccgctgtgtaccccgagcatctgggaggtggggagcgggctggggagaagaacacctggagcggaggttgggatcagggagggccgcagtcccggtaccaccaccacctgctgtgggacctgcagtctcctcatcagcagaacagctgtgaagccatcctgcccgtccacagggtggtgggttgtgaaggctgcatacctggcagagctggagaagctctggggagatgctggacatgcacgctaggagtggtttccctgccttgcccagactctgctcccatcacctgaacctccctgtcaccaccacggaactgctgtgaccattgctttcttcttaagcagattaacagacatctcctgccccaccccgccaaacaaacaaatgaacaaacaaaaaacgtgcttgaaggagtatgaacttatacagtcttttctaaacactgttaagtgctggtattgggatcttcttttaaaatgaaccatattccccaggctttggatgacactcatggttgcccaccctccaacttccttccctgctggcagaaccctgggtttgttttcgttccacccccgaccccactgcattcctgactcaggcaaatctgcagggtccagtgcagtcagggggccacgttccctcctccaacgggtgctgaggtcgctgcttgattggatgctgctgatgacctgcgaggaggagggcgccagggcacttttgggactttgctcttctgaagagatgcttccacagcacggtcgcagtcacgtcttgatgtgatgtctggaatggtggtggccgtcttgtggctgtgagaacaggctgaggttgattggatggaaggaaggaaggatccttgttcttgacactgtctgtgagccttcaggttatcgccctggcaccacccagcccttggagtagacacctgtctactctacatactccatttggagttgggttttttggtcacttgcagttgaaagcaccctaactgatatacacaaactatttttagtgcgggtctgtgtttggcccttatggaagactttgggctgagctgcccatggtgagggagacggacttcgtgtcttcttaccactctgtgtcctggtggcttgtatgtgtctctgcccatgaggcaaaagcctaaagggcaagggcggattttcttaatcggatgttccttgcaccaagcacataggagacactcaacgaatggttgttgagagagttctctttcacggaggtggtgttttgtgaaacgatgctgccaggcctgcttgttatttgtctgttggttgtaatctgcatgaatgcaaagagccatctttaatcatgctgtggaccagcctcttccaaggtattagcatgactcccactacctgctcagcatcctgcctatggctaggactttgtaatttacatagatacgctggggagacagggagcccatgaccaggactctgacaccctcactggagctgtttctacatctaccctgggtggctgtctaggacattaggcgattcgtgtcttcctaaagtccctctgttgagagacttctggctctgttgagaggacactatttagcattgtgagtccctgcaggctgggggccagtgggcattttccttctagatgtcccctctcttcttctggcctcccaggcttcctgctcctgagactgtgagaactggcctgtgctgggctcactgcagaaagaccgtcgtctccaaaggtcttgtgccaaacttgagctacaagctctttagccgggcctgaggtctccgcctgggctctgggagagcagcagtggctgtttctgccccctcactgctgtcatgcccacacttcacttgcattttcttcgccccccagccgtgtgagaatctggtatgaggagtgggactcacgtgccctctttcttctcctcttccccttggccttttcatctgtcagtggaggacagatgtttgccccgtttacttctaggctcactgtggggctccagggagatggtgaagtggccaaggagaggagctgccaccttcaagacggcctgtggccagtgctgctttaaagggagactcagagatgctttgctgtgggtggcgcgggaaccagcatggggacagcagtgcagaggccttggactcagagtgcgtgggccccacggggcttcacggcgcctgtggctgtgcacttccagccttatctgtgctgcatctcctccacattcccctgtggagctgatgtctagacagctatggaattaaatgctcaattaccgagtaggaatttggccagcagaggtatagctgcggagcagacagactcgaggtgaggctcacggctgagaacgggccccacctggctctggaatgagctgaggggccccatgctcctgcagccagtggctcctgtggggagttggggcagtgacccccaaaaggcagtttgacctcatggagagccataaatctggcctggtcaccatctctgcaacacatcattccattgcaaagatttctgcctgtgattggaattctgggtgaacgtgtactgggcatgtgggtctgagagctgggaagcctgttctcttgtttagccaggctgcccatgggctgtgaggagtgcccccatctctgagcctcagtttccacatctttaaaatggggggaaaatacagctcaactcctaagggtgccgtgaaagtactttgtcacctgccaggcaaaggctcattcctttcacagaaatgcaaggtttacaatgtgagacccctccctacttcgccgcatgtgtccgcttgcttttttctgtcttagggttgccctacatgagctaggaaatgtctgagtgaataaaaacgtaaacgagatgatcactggtggtgcccattggtgcagcctttgcctaaatggccactacgtagccacattttctcgtctgtgttcaggtgaggactggttcctggggagactccctgggttcacattatgggtgtctatcttgtcgaagcccatatggtcacccaagtgtgactgaaccatggggtgctctgggccccatttttggcagcaggcagcatcccctggaggcctggccctccccaggagcatggagagcagtgcccatggacaagcagtctgcagcctccatctcctcctccctgcccggggggctcccccgccccagcctcgcagcttctccaaaagtgtttgtctccttgccgcatcctctgggcctgagctcagatggtggaaaagaagagctggaaggagagttgcctttcggtctgtctgccttctgaggtctcctgagacatacaggctgggcctgcctccctttctaggaggcgccgatgggtggtaaggataggggataagtgagatgtgaatgaggatcaccacagcaagccctgactcataactttttgatgggttttcaatgtgtggtgaagcaggcgcctgctgggcccccttcctgagttgagcttgatctcctgcctcctgtctgtctccttaggcagccaggctaccctgctccagcaacctgtgccaccccgtccctttacctgtcccaagcccagccccgaaggcctcaaaggcctggccttccagccagtccagggcctgaagggatggcagtgtccctggtggacctcccccagcatggcgtagcgcacatcccagccctgcctcctgccccgcctgcacgccatgaatgctgaagtcatgcctggcaggggctgctggcccgggcccagagtaaacaggctgcgctgagcttgctggtgtgctgctggatgctgatgagcttgaggagtgtgggaagtcagtgtggggccgagtagggatgctgcaggcctgcatctccccccagctgccctgcacgctccagcctcaggcaaccccacagggaaagggtcacccactgtcagggcagacctttaccatggctgggtgacatgggctggctgtgggaaggtggttggtggttccccctgttggatttgcacaggcccagatgctcacagcaaaactaacacctagatgatgcttataggagccagcgggtaatcaaagagctgttcagatcttcatttgcttcgttctcacagtggaccattgaggtagctgtatgttagtcccattttccagatgggaaaactgaggacctgagtggtcgtaagctcaggcccctatctaaatcacacagcctggccccaggtctatgctcttgaccatggacagtgctctcctggtcctcttggtatctgtgatctgagggaccttcctcctcctcagtcttgtatagtcagttttaggtcttagactctttcttcacatccctttcttctttcgggagctctctcacccagcaccttccttatctagtatgtgttgggggatatttgtggcatgatgtggcgctgtgtagtggatgagagagtctgtttttccggtttcagccccaggtttcaatccctgctctgtctcaagtcacccagactcttggaggctcagtttcctcatctgttaaatgggcatggtggtcacctcacctcatcagctggtgtctgctccatccctggtggaggagatggctcaagtaaccccttggttccacctgccccaccccactggtcccctggctctttcttttttgagatagacaaacgtgaggctctggatttgcagttcccacgagggctggggtggctgtctgctttctgggtctggtccatgttttccagggcagctgctcgttctaagtgaacaaaggctgaaggaactcaggaggtttgctcggctccgaggatggcagagagggaaggggtgccgatgccttccctgatagagctggggaggcccttctgtggttccccccagctccttggcttgggtgaccctggagctgacttctgttccattttgttgtgcagagttgtttggggctcctggctctgcctggcctttgtgggccactggagatcagggcttctggagttggccaattagcccgcccagcccagggagcacaggtgtctgatggagggccttttcaggagaggagagatggcccgcctgttgggtcttgctgtcttgggtcctggaggccttgctgtccccatgctccatccatgcccttgaccaatgtggccctgtactcagcataggcatgcacctgagtcagtgcaattccctgtccacagagcaccccaaatattccaggcctcaggatgggtgtgcacatgatgagccgggcaggtttcaccacctgtagcttgggatccttcccggggcttggttctcgaaggctgccccaggcagtcacaccccaaaccctaaattcatgttgtcttcctctgtctcttggcctcaaggtttcagagtgagtctgtgctgatagcttcaagatgtgatgagaccccgacttggcctccagttccctccccacggtttccttggcgtgtgtgcggcttcagtggtcactggctcccacacagcttgtaatgtgtggattacgggtgggagggaagtccggtcctgcctgcagcaaagggatgttagtcgtgagctcagttccccatcgggcctggtgtttccaaatggcccggcactgtccctgcttggttttccatgatatctgtgcctttacccatttggttaaattaaacaaattcagcaatgccagccattgtggtttcagggtaagttgcctgtcctgctgcttggccgctcctcagagctgcggccgggagagttcgtggtcgccatcggaagcccgttttcccttcaaaacacagtcaccaccgggatcgtgagcaccacccagcgaggcggcaaagagctggggctccggaactcagacatggactacatccagaccgacgccatcatcaacgtgagcctctgtccctctgcgggtggggcttggggcagggttttgccagaggagaggagtcagcatcggtctctgacttccttgtagtctgggtgaaaggatggaactagaccaagccatgtggatcctagtgccagcagcacgacaggggtcacacggcggggacagtgacactggagcaggtggacagccagcctcctcccaggaggaagaagttgtgttgggtgctttagggtgattgcagttggcttctgggcttcagagagaaaatctccccatttacggcacctctaaaactttctgaaaattgttaaggtcatttttttccagcaaaatattaggttaatgggaatgaatctcagagaagaatcatgccccacactgtagacaccatgctcaggagacggccaggcagggacatagattggaccacgttatgacacaatttgtaacctttccatttctgtttaattgcagtatggaaactcgggaggcccgttagtaaacctggtaacgtattttaaacgttatgtcgtttgtttttatttatgtacacactgtttttgttttgttttgttttttgatgtagggggtcttttcaaacataagcttgccaaagcgtgttatcaagtttctttaaaatgagctctgtgaatgtactgcatgcttgcaaatgaccctatggatcttttctggaaagagtaaggcaggctggaggtgagggttggaaatgttatgccagagagcacacttgtgtctcagagttacaggtaaacacagtgaaattcagggccaatgcaggagtaaggtgaaggtcaccaaaagtgctggccggtcactgaaagagcctcctccaaattaaatctcctgggctgctgaaggagctggctgggctcatacacactttctcttggccaggaatcctcccttaaggcctggctggaatgaggaggagttacccacccacaaagatatcatttaagtctacccttaaatacttgagcagaaaaagtgaagccttagaacatagaccatcagcgctagagggcagctccggggccgttcatagagggcagctccggggccatttgtaggggccgtctttagtaaggccttggcatcaggtactgacatcccagcactcgtgggaagtgcgcacggggcgatgtatccccgcttggcagctttcccttcccagcagaggggcagctgtgctcccagctctgccctccgcctcccccgcagcaccctggggatggagtggagacggctttgcgggtaatgaagcatgacagccctaagctctagggttgttccccctcaagtcagcagagtcatcttaagatcattagaaatgagagaagcaggaaggtgtaggcagccacctagaggactctgagcctttggaaacgtattccttgtgaaacaggagcaaataatatcgtgcattttgaaactatctgtgcttaccgcgaggtgagcacccagtggcgacctggagtgtgtgcgattcttccacagctgcgcgtggcctacgctgcctgggtgtcctgatgcctctctccctgctcccccggggatcccctccatgcagctccccgcttcaatctctgaaatagctcagtgacttctttcatgcacattctctttgggggtgtacctgccggtaagccttcacgattcagcaagccgtgtccttcttgcctttcaggacggtgaagtgattggaattaacactttgaaagtgacagctggaatctcctttgcaatcccatctgataagattaaaaagtttctcaccgagtcccatgaccgacaggccaaaggtaggcaaggcccacatagccccggggactccggagattcggcctgaagctcaactgccctttgggaattggggaagggaaaagtggcagcccctaagactagccaagccgtcttcgatccagaagtgaacaggaatgcacattactaaatccctggtagaaggtcacagacattgcgccatttttgtcctccgatcatgacaatgtcacttgagtcagtctaatatgtaccagacacgatcctaggtgatttctgtccattatttcactttatttatgtatgttacttaattcttttgccctatcagttaggaattactagtcccattttgctgatgagaaaacaggttcagggagatcattctacaaacatttattgcctaagtcaagcagggagcttggcagtagactgcccaactggagcctggggctccgctgaggcctttgccggtgtgtgtttatgttctgttgggggatgggaaggctgacagtaaataatcagacacattagatactattagtgctcccaagaaaacagatcagggtggctggcaagggagtgactggacaggcagttggtagagatggtgtggccaggaaatgcctcccaaactgaggtctgagtgaggaggagccagcaggtagggatgtggggggaacactccagaaggaaagacagaggactcagcatagctgagtgagcacaaggcccctggagtggcctgggggccggagcacagtgacagcatggaggtctctggggtggaaagctcgccaaggccaagcaagcaggctcacagcgggccatggtgaggggcctgggttgcatcctaaccgcatttaagaacagggaagttcatgatctgattgatgtcactgaaaggacactctgatggttggggggagtctgctggaggagttgctggaagttggggaccggagaaggagctctcccagtcatctggatgagacacgctgggggctcagacaagggtggtggcagtggaggtgggacagaggggtcacattccaggtatacatgggggtagcgcaagcctggggaagggccagctgtcaggatgaggccatgaggaattgaggatcatgcccaggtatctgaccattaactgaacgatgagactttcctgaggtcccccagaggggaggggtccaaaccaggattcgagccgcaacctccgtgtgcccttctgtggcccttcctgcaacctgggggattgggcccccagcccctggtgtccccagcatacccaccaactgggctgaccttctgccgtccctttgttgtctcaccaggaaaagccatcaccaagaagaagtatattggtatccgaatgatgtcactcacgtccaggtgggcaaacaggatgcgtgtgtgtgtcttaaattttaataaacctgaacttcagaaggtgctcacgggcacccctgaaagagaaagcttatgcagccttaagacatctcagtttctgcttataatgaagtagcatcaggaaagaggacaggtcatcagccgtggcccctttgtttggttttatcctgtgtttctgcattctgagctggttttcttcattggcggctggccctccagtgtagaaggttctgccctcctctttgaaggcaggcctgagcagtgcgtgtgtggtggggctgttgattcattctggctcatgtcttccttaccccatattctgttgaaacccacattccaggagggccccaagcccctcccacagctctaggcactctgctttcattgctctgctctgcggcagctcgtgggccgtggctgcaggaatgccagggcaggcccagtgcagggaagtgaatgactgatgtgcttgttttccccgagctggtggaactgcggcctgtggttggcaggctcacggcatcctggtgttttaacctggatgaaaaattctggtgtaatctcgtgagtcctggtagtatagactcaactggcgtggctgaaactgtcagaggtaaagtaggaaaagactagaatatactaacaggtagattaatgtgttcattactatgatgaattaatgattcactcactgtgaaagtattaatatattttgatacacattatgaatgatggtccctttcttcgcactccagaagatggagccacttgtcaaggttaaagtgtctcctcagttgtttgcctttggaactagaaggtggagggaaagatgggaggcccttggcgcccagctccctgggttcctgttccagctctgatacttcctgccttgtgaccttgggaacgatatgacccctgagtgcctcagtttcctcctcttcaggatggggatgacagcgcaggtgcttctggtggtagcggtgatcaccagccacagtgatgccagtcactatctaggccgggtgctttactggggtgacctcatctgatcctcacaactcatattgtagggtactgttattatccccgtttcgcaggtgaggaaatgaaggcacagagaggttaagcaaccgtctggggtcacgcagctagcaaatagcagagctagggctacaaaccaggccaaccactatactttacggactccttagtaatagctactgttaattaagaaataataacaatgatgatggctgcgcattgctggctcacacctgtaatcccagcactttgggaggctgaggcgggcagatcagttgaggccaggagttggagatcagcctggccaatttgtgaaaccctgtctctactaaaaatatgaaaaatttagctgggcttggtggcaggcacctgtaatctcagctactcgggtggctgaggcaggagaattgcttgaacccaggaaatagaggttgcagtgaactgagatcgtgccattgcactccagcctgggtgatagagcaagactctgtctcaaaaaaaaaaaaaaagaaaagaaaagaaaagaaataataataatgatgaaagcactttccttgctgttaccaagtaaatctttgactctggtagagaggcaattttaaaataggatcagaactcctggaggaattttacattagacccagggagaagaagggaactggtgagagcttgagttttgcctggggaaggactggtgtctcttcacactaacacgggtgctttttctctggagcagcaaagccaaagagctgaaggaccggcaccgggacttcccagacgtgatctcaggagcgtatatcattgaagtaattcctgataccccagcagaagcgtgagttagagtcattttcccttattttcccttttcctaatattcttgttgctcctgtaggggtagcaggaagagggagcgctgttccttttctactggctcagatgacagtgttgatccttgacagatgtggtcggacgttgctggtcattcctgctggccaggccttctgacctggctcggcttgggactcatccataggagggtgccttctgtcttcaaaagtccttgctccactaggaccctccagatggacagagcaatagcagactcataatgagtctctggctggccagagagagggtttcaggaacagtgtccccaagccctcacgtggtggtcctgttctaggcttcgggacccttctcctcctggagtcttccagattgtctctgacagttaggcccatacctgtcaacacctccagaaaaataacccaagtgatatcaaagtaacatgacaagaagtagctcaaccatccatcagggtttgttacctgtattggaatatccagaaaaaagtgctagaccaggggccagcaattgtgccctggggctggatctggcccactgcctgcttttatatggagctgtggactaagaataatttttgcattttatttctatttttacttattttttaaattttttattttcataggttttgggggaacaggttgtatttggttacatgaataagttctttggtggtgatttgtgagattttggtgcacccatcacccaagcagtatacactgaacccaatttgtagtcttttatccctcacccctgtcccagcctttcccattgagtccccagagtccattgtataattcttatgcctttgtatcctcatagtttagctcccacttatgagtgagaacatttaaatagttgaaaaaatcctgaaataagaatagcattttgtgacttgttatatttgtatgcaattcaaatttcagcgtccactgaaatttggtttatgacatctttggtggcttttgtgctggagcagccgagttgagtagcttcaacagagaccatatatacggcaaagcctaaaatatttcctatggacctctttacagaaaaagtttgcagacccttatgctggcccatatgaaggaccatgacagcgttttgacgctgacctatataagagctacagttatagtggcaaccacacaaaggaagtgcctcttaacagaagcattctgcccacccttgtaggaactgcattctgagttgcaataccctttataagcaagttggccatggtcacgctacatggcagatagtacctggtacatccttccccactttggggtcaatcttgacctttgatctccttggggtcataaggccatacaagtgttagtaggcatttctagagtggacataatggatgagttagcctaaaaatctcaaaaggagcccagcatcatggcacctgcttgtaatcccagctattcaggaggctggagcagaaggatcccttgagcccaggagttcaagactagcttgggcaacaaatgagaccccatctcaaagaaaaaaaaaaaggtgggggaagaacattataataataataataataataataaaaaccttgataagtatccagtctaccaatggtttattttttattttattattattattttttgagatggaatctcactctgttgcccaggctggagtgcagtggcaaaatcttggcttactgcaacctccacctcctgggttcaagtgaatctcttgcctcagcctctgagtagctgggattacaggtgcccaccaccaaacctggctcttttgttttgtaattttagtagaaccagggctttgccatgttggccaggctggtcttgaactcctgacctcaggtcatccacctgcctcagcctcccaaagtgctaggattacaggcatgagccactgtgcccggcccactgatggtttgaattattctaagttcaccaccatccaatcctgtttgctctgggcttttaggttctaaactgtgcctctgtccatgtaaagtcagatcaggaggaatggagacatgaaacattgctattgtgtttccctttgtgttgcagtggtggtctcaaggaaaacgacgtcataatcagtatcaatggacagtcggtggtctccgccaatgacgtcagcgatgtcattaaaagggaaagcaccctgaacatggtggtccgtaggggtaacgaagacatcatgatcacagtgattcccgaagaaattgacccataggcagaggcatgagctggacttcatgtttccctcaaagactctcccgtggatgacggatgaggactctgggctgctggaataggacactcaagacttttgaccgccattttgtttgttcagtggagactccctggccaacagaatccttcttgatagtttgcaggcaaaacaaatgtaatgctgcagatccgcaggcagaagctctgcccttctgtatcctatgtatgcagtgtgctttttcttgccagcttggtccattcttgcttagacagccagcatttgtctcctcctttaactgagtcatcatcttagaccaactaatgcagtcgatacaatgcgtagatagaagaagccccacgggagccgggatgggacggggcgcgtttgtgcttttctccaagtcagcacccaaaggtcaatgcacagagaccccgggtgggtgaacactggcttctgaaatggccagagttgactcttttaggaatctctttggaactgggagcacgatgactctgagtttgagctattaaagtacttcttacacattg

SEQ ID NO: 7 - DNA, 22 bp, Artificial
  Oligo target sequence
ccaacaaccaggtaaatatttg

SEQ ID NO: 8 - DNA, 18 bp, artificial
  Oligonucleotide sequence
caaatatttacctggttg

SEQ ID NO: 9 - DNA, 16 bp, Artificial
  Oligonucleotide sequence
tttacctggttgttgg

SEQ ID NO: 10 - DNA, 18 bp, Artificial
  Oligonucleotide sequence
ccaaatatttacctggtt

SEQ ID NO: 11 - DNA, 20 bp, Artificial
  Oligonucleotide sequence
ccaaatatttacctggttgt

SEQ ID NO: 12 - DNA, 18 bp, Artificial
  Oligonucleotide sequence
atatttacctggttgttg

SEQ ID NO: 13 - DNA, 16 bp, Artificial
  Oligonucleotide sequence
tatttacctggttgtt

SEQ ID NO: 14 - DNA, 16 bp, Artificial
  Oligonucleotide sequence
atatttacctggttgt

SEQ ID NO: 15 - DNA, 17 bp, Artificial
  Oligonucleotide sequence
atatttacctggttgtt

SEQ ID NO: 16 - DNA, 275 bp, Homo sapiens
gacagtcagcatttgtctcctcctttaactgagtcatcatcttagtccaactaatgcagtcgatacaatgcgtagatagaagaagccccacgggagccaggatgggactggtcgtgtttgtgcttttctccaagtcagcacccaaaggtcaatgcacagagaccccgggtgggtgagcgctggcttctcaaacggccgaagttgcctcttttaggaatctctttggaattgggagcacgatgactctgagtttgagctattaaagtacttcttac

SEQ ID NO: 17 - DNA, 18 bp, Artificial
  Oligonucleotide sequence
cttcttctatctacgcat

SEQ ID NO: 18 - DNA, 16 bp, Artificial
  Oligonucleotide sequence
tactttaatagctcaa

SEQ ID NO: 19 - DNA, 16 bp, Artificial
  Oligonucleotide sequence
ttctatctacgcattg